(19) United States
(12) Patent Application Publication
(10) Pub. No.: US 2023/0126868 A1
(43) Pub. Date: Apr. 27, 2023

(54) ORGANIC ELECTROLUMINESCENT ELEMENT AND ELECTRONIC DEVICE

(71) Applicant: IDEMITSU KOSAN CO.,LTD., Tokyo (JP)

(72) Inventors: Emiko KAMBE, Tokyo (JP); Masatoshi SAITO, Tokyo (JP); Masato NAKAMURA, Tokyo (JP)

(73) Assignee: IDEMITSU KOSAN CO.,LTD., Tokyo (JP)

(21) Appl. No.: 17/801,120

(22) PCT Filed: Feb. 22, 2021
(86) PCT No.: PCT/JP2021/006688

§ 371 Date: Aug. 19, 2022

(30) Foreign Application Priority Data
Feb. 25, 2020 (JP) 2020-029481

(51) Int. Cl. H01L 51/00

(52) U.S. Cl. CPC H01L 51/0067; H01L 51/0052; H01L 51/0058; H01L 51/0056; H01L 51/508

(57) ABSTRACT

An organic electroluminescence device includes: an emitting layer between an anode and a cathode; and a first layer between the cathode and the emitting layer, in which the first layer has a thickness of 50 nm or more, the first layer contains a compound represented by a formula (1) below, and the first layer contains no metal doping material. In the formula (100), A is a substituted or unsubstituted fused aryl group having 13 to 50 ring carbon atoms, or a substituted or unsubstituted fused heterocyclic group having 14 to 50 ring atoms.

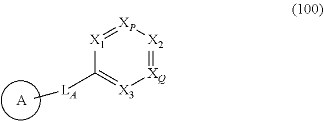

### TECHNICAL FIELD

The present invention relates to an organic electroluminescence device and an electronic device.

### BACKGROUND ART

An organic electroluminescence device (hereinafter, occasionally referred to as “organic EL device”) has found its application in a full-color display for mobile phones, televisions and the like. When a voltage is applied to an organic EL device, holes and electrons are injected from an anode and a cathode, respectively, into an emitting layer. The injected holes and electrons are recombined in the emitting layer to form excitons. Specifically, according to the electron spin statistics theory, singlet excitons and triplet excitons are generated at a ratio of 25%:75%.

Various studies have been made for compounds to be used for the organic EL device and a structure of the organic EL device in order to enhance the performance of the organic EL device. The performance of the organic EL device is evaluable in terms of, for instance, luminance, emission wavelength, chromaticity, emission efficiency, drive voltage, and lifetime.

For instance, Patent Literature 1 describes Examples in which a compound having an anthracene structure and a benzimidazole structure is used as an electron transporting material of an organic EL device.

For instance, Patent Literature 2 describes Examples in which a compound having an anthracene structure and a triazine structure, a compound having a fluorine structure and a triazine structure, or the like is used as an electron transporting material of an organic EL device.

For instance, Patent Literature 3 describes Examples in which a compound having a heteroaryl structure and a triazine structure, or the like is used as an electron transporting material of an organic EL device.

### CITATION LIST

### Patent Literature(s)

Patent Literature 1: WO 2010/134350 A

Patent Literature 2: WO 2019/163824 A

Patent Literature 3: WO 2019/163825 A

### SUMMARY OF THE INVENTION

### Problems to be Solved by the Invention

An object of the invention is to provide an organic electroluminescence device drivable at a low voltage even if an electron transporting material in a thickened electron transporting zone is not doped with active metal, and an electronic device including the organic electroluminescence device.

### Means for Solving the Problem(s)

According to an aspect of the invention, there is provided an organic electroluminescence device including: an emitting layer between an anode and a cathode; and a first layer between the cathode and the emitting layer, in which the first layer has a thickness of 50 nm or more, the first layer contains a compound represented by a formula (100) below, and the first layer contains no metal doping material.

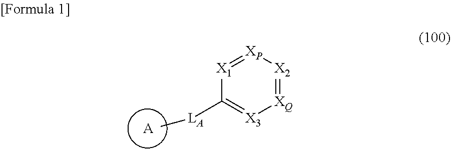

In the formula (100):

A is a substituted or unsubstituted fused aryl group having 13 to 50 ring carbon atoms, or a substituted or unsubstituted fused heterocyclic group having 14 to 50 ring atoms;

L_{A} is a single bond, a substituted or unsubstituted arylene group having 6 to 30 ring carbon atoms, or a substituted or unsubstituted divalent heterocyclic group having 5 to 30 ring atoms;

X₁, X₂ and X₃ are each independently a nitrogen atom or CR₃;

X_{P} is a nitrogen atom or CR₁;

X_{Q} is a nitrogen atom or CR₂;

at least one of X₁, X₂, X₃, X_{P} or X_{Q} is a nitrogen atom;

at least one combination of adjacent two or more of R₁, R₂ and R₃ are mutually bonded to form a substituted or unsubstituted monocyclic ring, mutually bonded to form a substituted or unsubstituted fused ring, or not mutually bonded;

R₁, R₂ and R₃ not forming the substituted or unsubstituted monocyclic ring and not forming the substituted or unsubstituted fused ring are each independently a hydrogen atom, a substituted or unsubstituted aryl group having 6 to 30 ring carbon atoms, or a substituted or unsubstituted heterocyclic group having 5 to 30 ring atoms; and

when a plurality of R₃ are present, the plurality of R₃ are mutually the same or different.

According to another aspect of the invention, there is provided an organic electroluminescence device including: an emitting layer between an anode and a cathode; and a first layer between the cathode and the emitting layer, in which the first layer has a thickness of 50 nm or more, the first layer contains a compound represented by a formula (1) below, and the first layer contains no metal doping material.

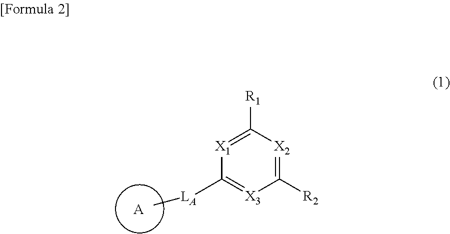

In the formula (1):

A is a substituted or unsubstituted fused aryl group having 13 to 50 ring carbon atoms, or a substituted or unsubstituted fused heterocyclic group having 14 to 50 ring atoms;

L_{A} is a single bond, a substituted or unsubstituted arylene group having 6 to 30 ring carbon atoms, or a substituted or unsubstituted divalent heterocyclic group having 5 to 30 ring atoms;

X₁, X₂ and X₃ are each independently a nitrogen atom or CR₃;

at least one of X₁, X₂ or X₃ is a nitrogen atom;

at least one combination of adjacent two or more of R₁, R₂ and R₃ are mutually bonded to form a substituted or unsubstituted monocyclic ring, mutually bonded to form a substituted or unsubstituted fused ring, or not mutually bonded;

R₁, R₂ and R₃ not forming the substituted or unsubstituted monocyclic ring and not forming the substituted or unsubstituted fused ring are each independently a hydrogen atom, a substituted or unsubstituted aryl group having 6 to 30 ring carbon atoms, or a substituted or unsubstituted heterocyclic group having 5 to 30 ring atoms; and

when a plurality of R₃ are present, the plurality of R₃ are mutually the same or different.

According to still another aspect of the invention, an electronic device including the organic electroluminescence device according to the above aspect of the invention is provided.

According to the above aspects of the invention, an organic electroluminescence device drivable at a low voltage even if an electron transporting material in a thickened electron transporting zone is not doped with active metal can be provided. According to the above aspect of the invention, an electronic device including the organic electroluminescence device can be provided.

### BRIEF EXPLANATION OF DRAWINGS

FIG. 1 schematically shows an exemplary arrangement of an organic electroluminescence device according to an exemplary embodiment of the invention.

FIG. 2 schematically shows another exemplary arrangement of the organic electroluminescence device according to the exemplary embodiment of the invention.

FIG. 3 schematically shows still another exemplary arrangement of the organic electroluminescence device according to the exemplary embodiment of the invention.

FIG. 4 schematically shows a further exemplary arrangement of the organic electroluminescence device according to the exemplary embodiment of the invention.

### DESCRIPTION OF EMBODIMENT(S)

### Definitions

Herein, a hydrogen atom includes isotopes having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Herein, the ring carbon atoms refer to the number of carbon atoms among atoms forming a ring of a compound (e.g., a monocyclic compound, fused-ring compound, cross-linking compound, carbon ring compound, and heterocyclic compound) in which the atoms are bonded to each other to form the ring. When the ring is substituted by a substituent(s), carbon atom(s) contained in the substituent(s) is not counted in the ring carbon atoms. Unless otherwise specified, the same applies to the “ring carbon atoms” described later. For instance, a benzene ring has 6 ring carbon atoms, a naphthalene ring has 10 ring carbon atoms, a pyridine ring has 5 ring carbon atoms, and a furan ring has 4 ring carbon atoms. Further, for instance, 9,9-diphenylfluorenyl group has 13 ring carbon atoms and 9,9′-spirobifluorenyl group has 25 ring carbon atoms.

When a benzene ring is substituted by a substituent in a form of, for instance, an alkyl group, the number of carbon atoms of the alkyl group is not counted in the number of the ring carbon atoms of the benzene ring. Accordingly, the benzene ring substituted by an alkyl group has 6 ring carbon atoms. When a naphthalene ring is substituted by a substituent in a form of, for instance, an alkyl group, the number of carbon atoms of the alkyl group is not counted in the number of the ring carbon atoms of the naphthalene ring. Accordingly, the naphthalene ring substituted by an alkyl group has 10 ring carbon atoms.

Herein, the ring atoms refer to the number of atoms forming a ring of a compound (e.g., a monocyclic compound, fused-ring compound, cross-linking compound, carbon ring compound, and heterocyclic compound) in which the atoms are bonded to each other to form the ring (e.g., monocyclic ring, fused ring, and ring assembly). Atom(s) not forming the ring (e.g., hydrogen atom(s) for saturating the valence of the atom which forms the ring) and atom(s) in a substituent by which the ring is substituted are not counted as the ring atoms. Unless otherwise specified, the same applies to the “ring atoms” described later. For instance, a pyridine ring has 6 ring atoms, a quinazoline ring has 10 ring atoms, and a furan ring has 5 ring atoms. For instance, the number of hydrogen atom(s) bonded to a pyridine ring or the number of atoms forming a substituent are not counted as the pyridine ring atoms. Accordingly, a pyridine ring bonded to a hydrogen atom(s) or a substituent(s) has 6 ring atoms. For instance, the hydrogen atom(s) bonded to carbon atom(s) of a quinazoline ring or the atoms forming a substituent are not counted as the quinazoline ring atoms. Accordingly, a quinazoline ring bonded to hydrogen atom(s) or a substituent(s) has 10 ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group. Herein, “YY” is larger than “XX,” “XX” representing an integer of 1 or more and “YY” representing an integer of 2 or more.

Herein, “XX to YY atoms” in the description of “substituted or unsubstituted ZZ group having XX to YY atoms” represent atoms of an unsubstituted ZZ group and do not include atoms of a substituent(s) of the substituted ZZ group. Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s). The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent. Similarly, the term “substituted” used in a “BB group substituted by AA group” means that at least one hydrogen atom in the BB group is substituted with the AA group.

### Substituents Mentioned Herein

Substituents mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G1B) below. (Herein, an unsubstituted aryl group refers to an “unsubstituted aryl group” in a “substituted or unsubstituted aryl group,” and a substituted aryl group refers to a “substituted aryl group” in a “substituted or unsubstituted aryl group.” A simply termed “aryl group” herein includes both of an “unsubstituted aryl group” and a “substituted aryl group.”

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by further substituting a hydrogen atom bonded to a carbon atom of a skeleton of a “substituted aryl group” in the specific example group G1B below, and a group derived by further substituting a hydrogen atom of a substituent of the “substituted aryl group” in the specific example group G1B below.

⋅ Unsubstituted Aryl Group (Specific Example Group G1A): phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, perylenyl group, and a monovalent aryl group derived by removing one hydrogen atom from cyclic structures represented by formulae (TEMP-1) to (TEMP-15) below.

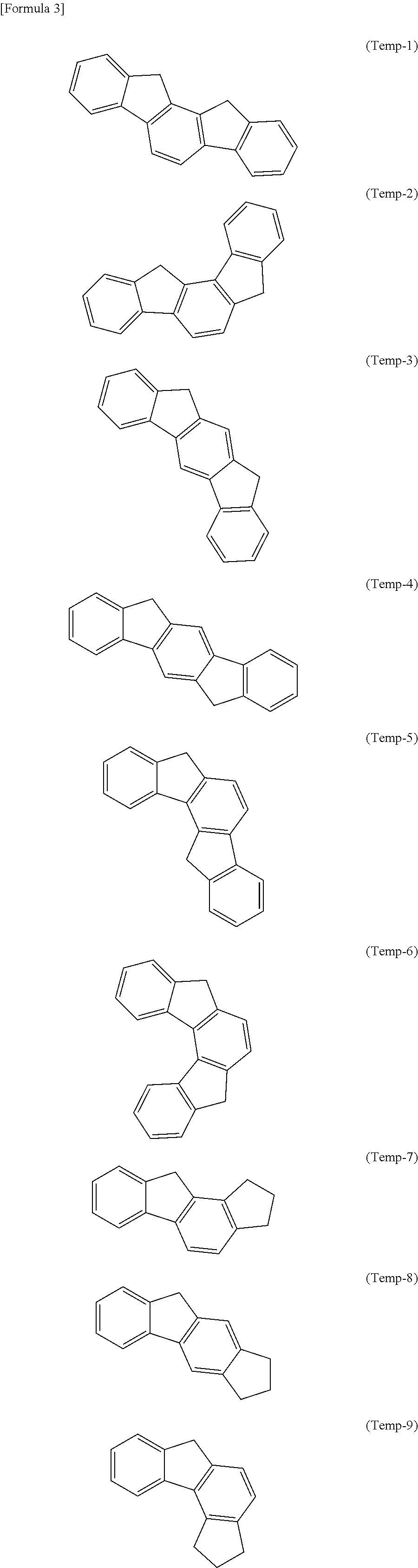

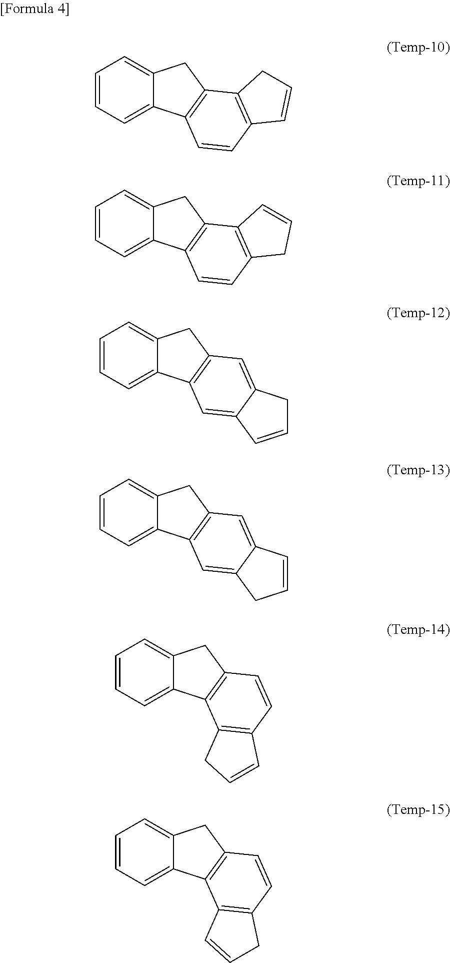

### Substituted Aryl Group (Specific Example Group G1B):

o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethylphenyl group, 9,9-dimethylfluorenyl group, 9,9-diphenylfluorenyl group, 9,9-bis(4-methylphenyl)fluorenyl group, 9,9-bis(4-isopropylphenyl)fluorenyl group, 9,9-bis(4-t-butylphenyl)fluorenyl group, cyanophenyl group, triphenylsilylphenyl group, trimethylsilylphenyl group, phenylnaphthyl group, naphthylphenyl group, and a group derived by substituting at least one hydrogen atom of a monovalent group derived from one of the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one hetero atom in the ring atoms. Specific examples of the hetero atom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B). (Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”) A simply termed “heterocyclic group” herein includes both of “unsubstituted heterocyclic group” and “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by further substituting a hydrogen atom bonded to a ring atom of a skeleton of a “substituted heterocyclic group” in the specific example group G2B below, and a group derived by further substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

### Unsubstituted Heterocyclic Groups Including Nitrogen Atom (Specific Example Group G2A1):

pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, pyridyl group, pyridazynyl group, pyrimidinyl group, pyrazinyl group, triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

### Unsubstituted Heterocyclic Groups Including Oxygen Atom (Specific Example Group G2A2):

furyl group, oxazolyl group, isoxazolyl group, oxadiazolyl group, xanthenyl group, benzofuranyl group, isobenzofuranyl group, dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

### Unsubstituted Heterocyclic Groups Including Sulfur Atom (Specific Example Group G2A3):

thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).

Monovalent Heterocyclic Groups Derived by Removing One Hydrogen Atom from Cyclic Structures Represented by Formulae (TEMP-16) to (TEMP-33) (Specific Example Group G2A4):

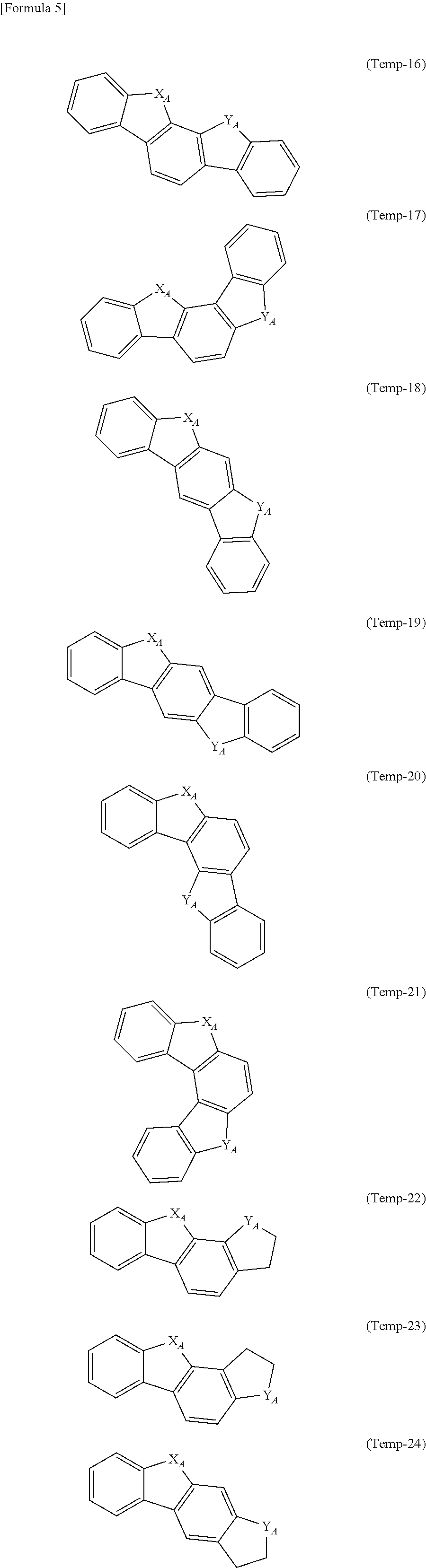

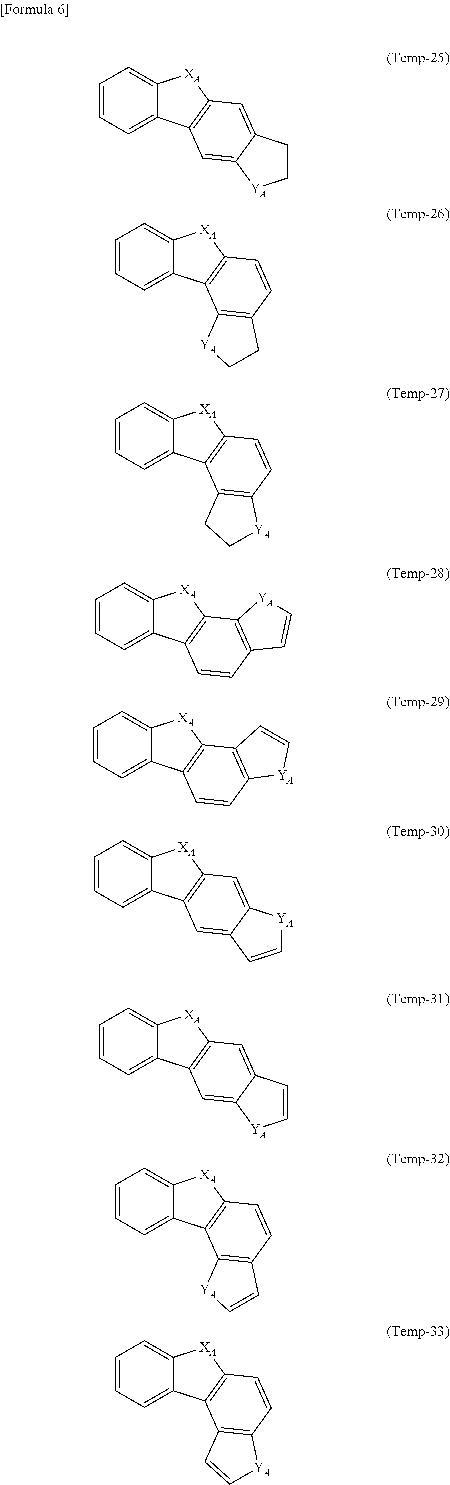

In the formulae (TEMP-16) to (TEMP-33), X_{A} and Y_{A} are each independently an oxygen atom, a sulfur atom, NH, or CH₂, with a proviso that at least one of X_{A} or Y_{A} is an oxygen atom, a sulfur atom, or NH.

When at least one of X_{A} or Y_{A} in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH or CH₂.

### Substituted Heterocyclic Groups Including Nitrogen Atom (Specific Example Group G2B1):

(9-phenyl)carbazolyl group, (9-biphenylyl)carbazolyl group, (9-phenyl)phenylcarbazolyl group, (9-naphthyl)carbazolyl group, diphenylcarbazole-9-yl group, phenylcarbazole-9-yl group, methylbenzimidazolyl group, ethylbenzimidazolyl group, phenyltriazinyl group, biphenylyltriazinyl group, diphenyltriazinyl group, phenylquinazolinyl group, and biphenylquinazolinyl group.

### Substituted Heterocyclic Groups Including Oxygen Atom (Specific Example Group G2B2):

phenyldibenzofuranyl group, methyldibenzofuranyl group, t-butyldibenzofuranyl group, and monovalent residue of spiro[9H-xanthene-9,9′-[9H]fluorene].

### Substituted Heterocyclic Groups Including Sulfur Atom (Specific Example Group G2B3):

phenyldibenzothiophenyl group, methyldibenzothiophenyl group, t-butyldibenzothiophenyl group, and monovalent residue of spiro[9H-thioxanthene-9,9′-[9H]fluorene].

Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of X_{A} or Y_{A} in a form of NH, and a hydrogen atom of one of X_{A} and Y_{A} in a form of a methylene group (CH₂).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B) below. (Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.”) A simply termed “alkyl group” herein includes both of “unsubstituted alkyl group” and “substituted alkyl group.”

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by further substituting a hydrogen atom of a skeleton of the “substituted alkyl group” in the specific example group G3B, and a group derived by further substituting a hydrogen atom of a substituent of the “substituted alkyl group” in the specific example group G3B.

### Unsubstituted Alkyl Group (Specific Example Group G3A):

methyl group, ethyl group, n-propyl group, isopropyl group, n-butyl group, isobutyl group, s-butyl group, and t-butyl group.

### Substituted Alkyl Group (Specific Example Group G3B):

heptafluoropropyl group (including isomer thereof), pentafluoroethyl group, 2,2,2-trifluoroethyl group, and trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B). (Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.”) A simply termed “alkenyl group” herein includes both of “unsubstituted alkenyl group” and “substituted alkenyl group.”

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by further substituting a hydrogen atom of a skeleton of the “substituted alkenyl group” in the specific example group G4B with a substituent, and a group derived by further substituting a hydrogen atom of a substituent of the “substituted alkenyl group” in the specific example group G4B with a substituent.

### Unsubstituted Alkenyl Group (Specific Example Group G4A):

vinyl group, allyl group, 1-butenyl group, 2-butenyl group, and 3-butenyl group.

### Substituted Alkenyl Group (Specific Example Group G4B):

1,3-butanedienyl group, 1-methylvinyl group, 1-methylallyl group, 1,1-dimethylallyl group, 2-methylallyl group, and 1,2-dimethylallyl group.

### Substituted or Unsubstituted Alkynyl Group

Specific examples (specific example group G5) of the “substituted or unsubstituted alkynyl group” mentioned herein include unsubstituted alkynyl groups (specific example group G5A) below. (Herein, an unsubstituted alkynyl group refers to an “unsubstituted alkynyl group” in a “substituted or unsubstituted alkynyl group.”) A simply termed “alkynyl group” herein includes both of “unsubstituted alkynyl group” and “substituted alkynyl group.”

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

### Unsubstituted Alkynyl Group (Specific Example Group G5A):

ethynyl group.

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B). (Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.”) A simply termed “cycloalkyl group” herein includes both of “unsubstituted cycloalkyl group” and “substituted cycloalkyl group.”

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent.

### Unsubstituted Cycloalkyl Group (Specific Example Group G6A):

cyclopropyl group, cyclobutyl group, cyclopentyl group, cyclohexyl group, 1-adamantyl group, 2-adamantyl group, 1-norbornyl group, and 2-norbornyl group.

### Substituted Cycloalkyl Group (Specific Example Group G6B):

4-methylcyclohexyl group.

Group Represented by —Si(R₉₀₁)(R₉₀₂)(R₉₀₃)

Specific examples (specific example group G7) of the group represented herein by —Si(R₉₀₁)(R₉₀₂)(R₉₀₃) include: —Si(G1)(G1)(G1); —Si(G1)(G2)(G2); —Si(G1)(G1)(G2); —Si(G2)(G2)(G2); —Si(G3)(G3)(G3); and —Si(G6)(G6)(G6), where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3;

G6 represents a “substituted or unsubstituted cycloalkyl group” in the specific example group G6;

a plurality of G1 in —Si(G1)(G1)(G1) are mutually the same or different;

a plurality of G2 in —Si(G1)(G2)(G2) are mutually the same or different;

a plurality of G1 in —Si(G1)(G1)(G2) are mutually the same or different;

a plurality of G2 in —Si(G2)(G2)(G2) are mutually the same or different;

a plurality of G3 in —Si(G3)(G3)(G3) are mutually the same or different; and

a plurality of G6 in —Si(G6)(G6)(G6) are mutually the same or different.

### Group Represented by —O—(R₉₀₄)

Specific examples (specific example group G8) of a group represented by —O—(R₉₀₄) herein include: —O(G1); —O(G2); —O(G3); and —O(G6), where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3; and

G6 represents a “substituted or unsubstituted cycloalkyl group” in the specific example group G6.

### Group Represented by —S—(R₉₀₅)

Specific examples (specific example group G9) of a group represented herein by —S—(R₉₀₅) include: —S(G1); —S(G2); —S(G3); and —S(G6), where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3; and

G6 represents a “substituted or unsubstituted cycloalkyl group” in the specific example group G6.

Group Represented by —N(R₉₀₆)(R₉₀₇)

Specific examples (specific example group G10) of a group represented herein by —N(R₉₀₆)(R₉₀₇) include: —N(G1)(G1); —N(G2)(G2); —N(G1)(G2); —N(G3)(G3); and —N(G6)(G6), where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3;

G6 represents a “substituted or unsubstituted cycloalkyl group” in the specific example group G6;

a plurality of G1 in —N(G1)(G1) are mutually the same or different;

a plurality of G2 in —N(G2)(G2) are mutually the same or different;

a plurality of G3 in —N(G3)(G3) are mutually the same or different; and

a plurality of G6 in —N(G6)(G6) are mutually the same or different.

### Halogen Atom

Specific examples (specific example group G11) of “halogen atom” mentioned herein include a fluorine atom, chlorine atom, bromine atom, and iodine atom.

### Substituted or Unsubstituted Fluoroalkyl Group

The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms. An “unsubstituted fluoroalkyl group” has, unless otherwise specified herein, 1 to 50, preferably 1 to 30, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein include a group derived by further substituting at least one hydrogen atom bonded to a carbon atom of an alkyl chain of a “substituted fluoroalkyl group” with a substituent, and a group derived by further substituting at least one hydrogen atom of a substituent of the “substituted fluoroalkyl group” with a substituent. Specific examples of the “substituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms. An “unsubstituted haloalkyl group” has, unless otherwise specified herein, 1 to 50, preferably 1 to 30, more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein include a group derived by further substituting at least one hydrogen atom bonded to a carbon atom of an alkyl chain of a “substituted haloalkyl group” with a substituent, and a group derived by further substituting at least one hydrogen atom of a substituent of the “substituted haloalkyl group” with a substituent. Specific examples of the “unsubstituted haloalkyl group” include a group derived by substituting at least one hydrogen atom of the “alkyl group” (specific example group G3) with a halogen atom. The haloalkyl group is sometimes referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50, preferably 1 to 30, more preferably 1 to 18 carbon atoms.

### Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50, preferably 6 to 30, more preferably 6 to 18 ring carbon atoms.

### Substituted or Unsubstituted Trialkylsilyl Group

Specific examples of a “trialkylsilyl group” mentioned herein include a group represented by —Si(G3)(G3)(G3), G3 being the “substituted or unsubstituted alkyl group” in the specific example group G3. The plurality of G3 in —Si(G3)(G3)(G3) are mutually the same or different. Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by (G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, a-naphthylmethyl group, 1-a-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl)carbazolyl group ((9-phenyl)carbazole-1-yl group, (9-phenyl)carbazole-2-yl group, (9-phenyl)carbazole-3-yl group, or (9-phenyl)carbazole-4-yl group), (9-biphenylyl)carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

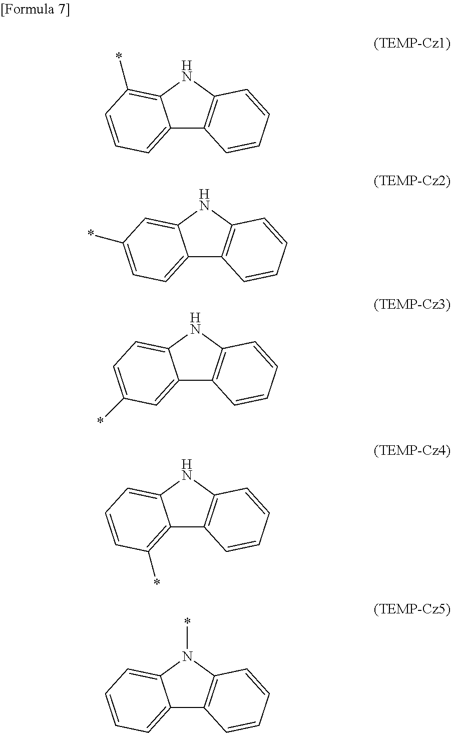 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

The (9-phenyl)carbazolyl group mentioned herein is, unless otherwise specified herein, specifically a group represented by one of formulae below.

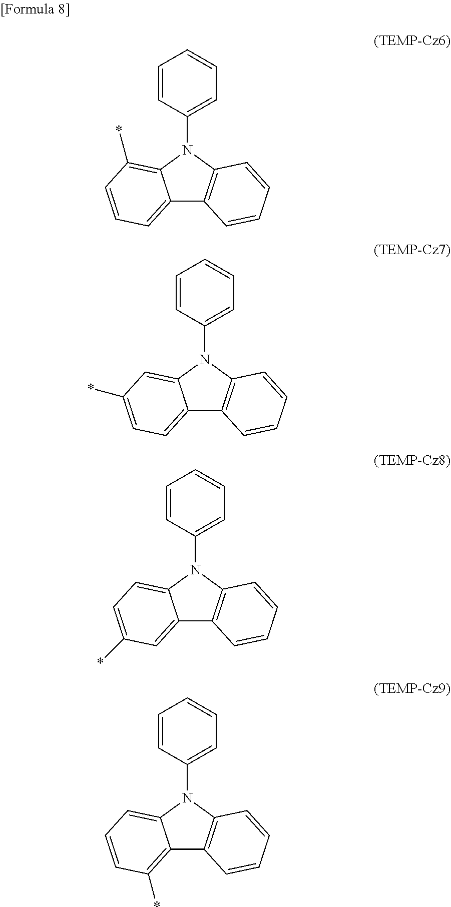

In the formulae (TEMP-Cz1) to (TEMP-Cz9), * represents a banding position.

The dibenzofuranyl group and dibenzothiophenyl group mentioned herein are, unless otherwise specified herein, each specifically represented by one of formulae below.

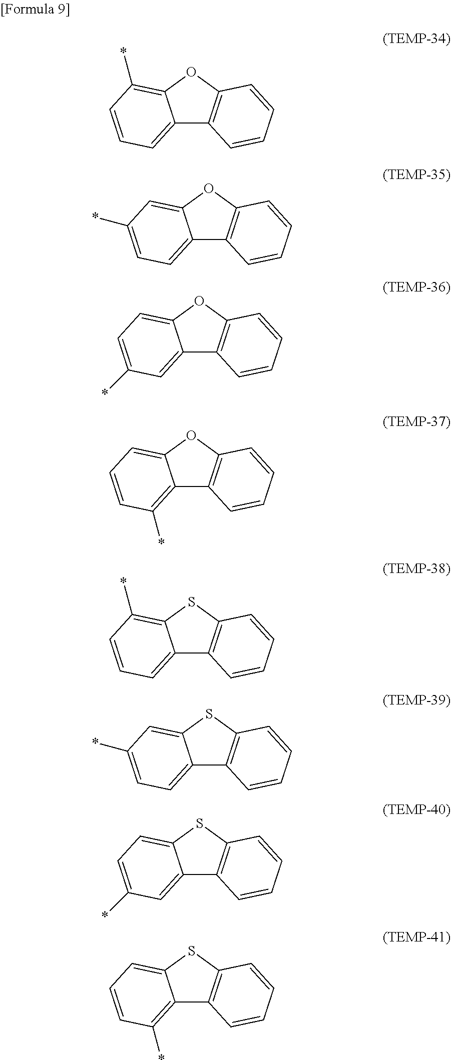

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.” Specific examples of the “substituted or unsubstituted arylene group” (specific example group G12) include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocycle of the “substituted or unsubstituted heterocyclic group.” Specific examples of the “substituted or unsubstituted divalent heterocyclic group” (specific example group G13) include a divalent group derived by removing one hydrogen atom on a heterocyclic ring of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived by removing one hydrogen atom on an alkyl chain of the “substituted or unsubstituted alkyl group.” Specific examples of the “substituted or unsubstituted alkylene group” (specific example group G14) include a divalent group derived by removing one hydrogen atom on an alkyl chain of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

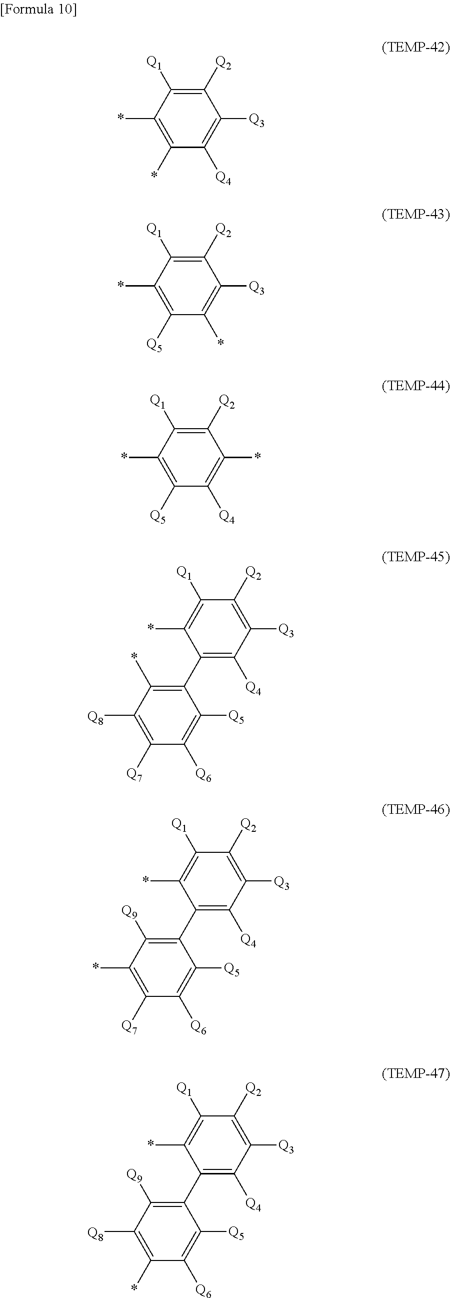

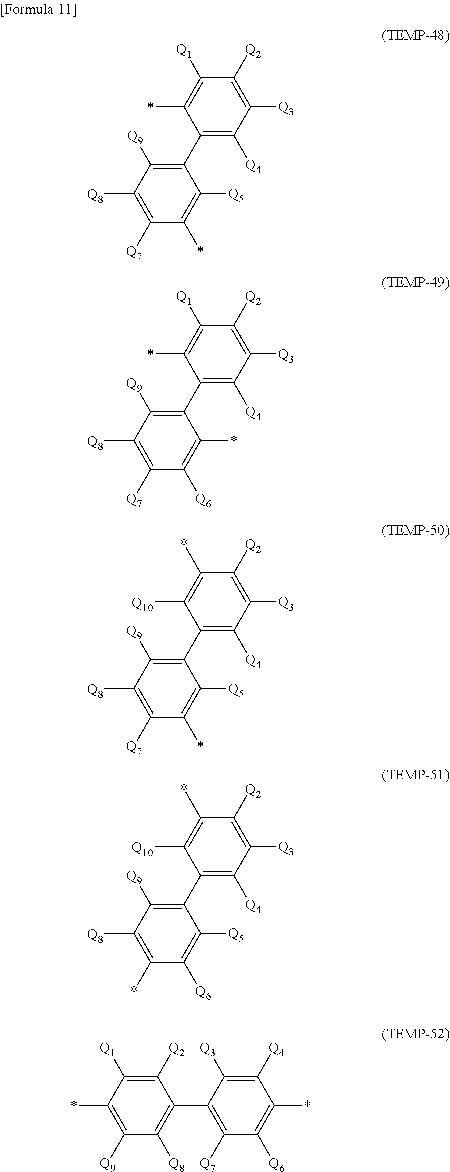

In the formulae (TEMP-42) to (TEMP-52), Q₁ to Q₁₀ are each independently a hydrogen atom or a substituent.

In the formulae (TEMP-42) to (TEMP-52), * represents a bonding position.

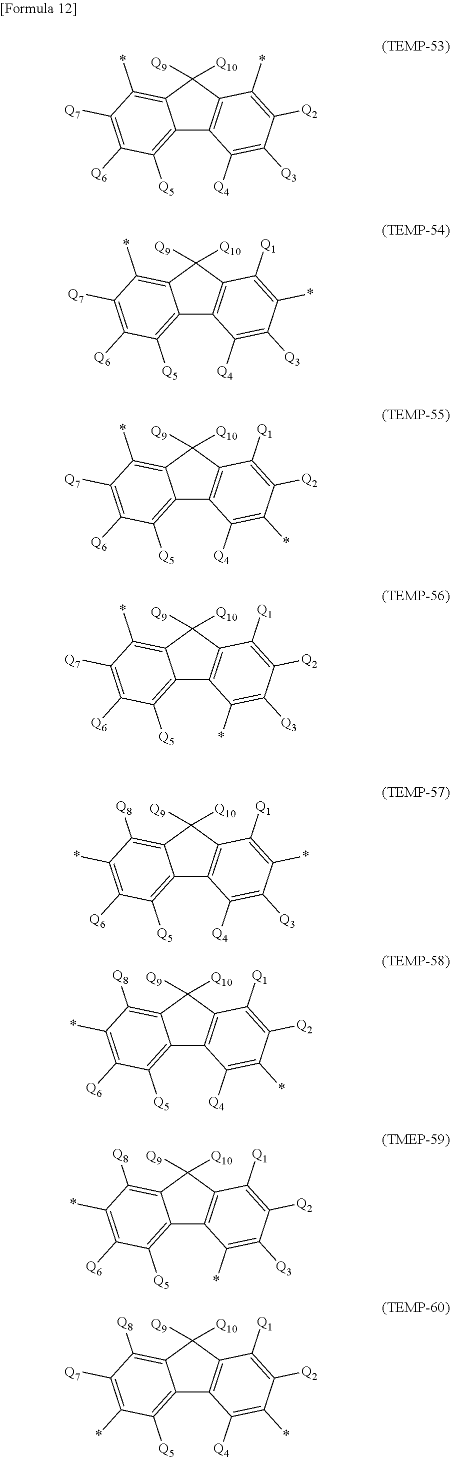

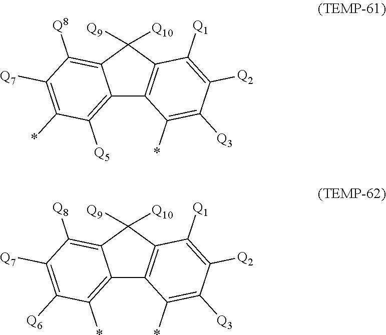

In the formulae (TEMP-53) to (TEMP-62), Q₁ to Q₁₀ are each independently a hydrogen atom or a substituent.

In the formulae, Q₉ and Q₁₀ may be mutually bonded through a single bond to form a ring.

In the formulae (TEMP-53) to (TEMP-62), * represents a bonding position.

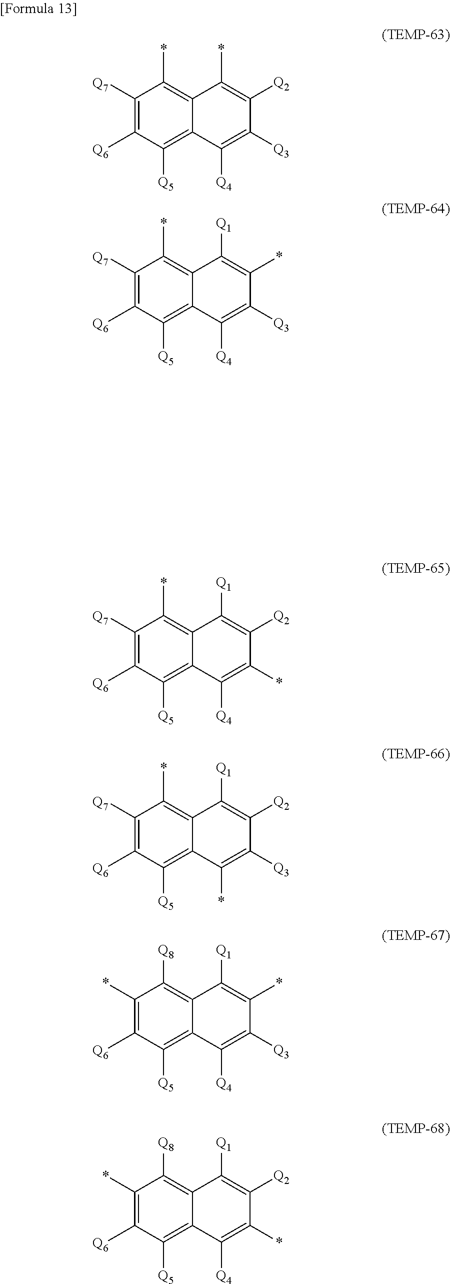

In the formulae (TEMP-63) to (TEMP-68), Q₁ to Q₈ are each independently a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

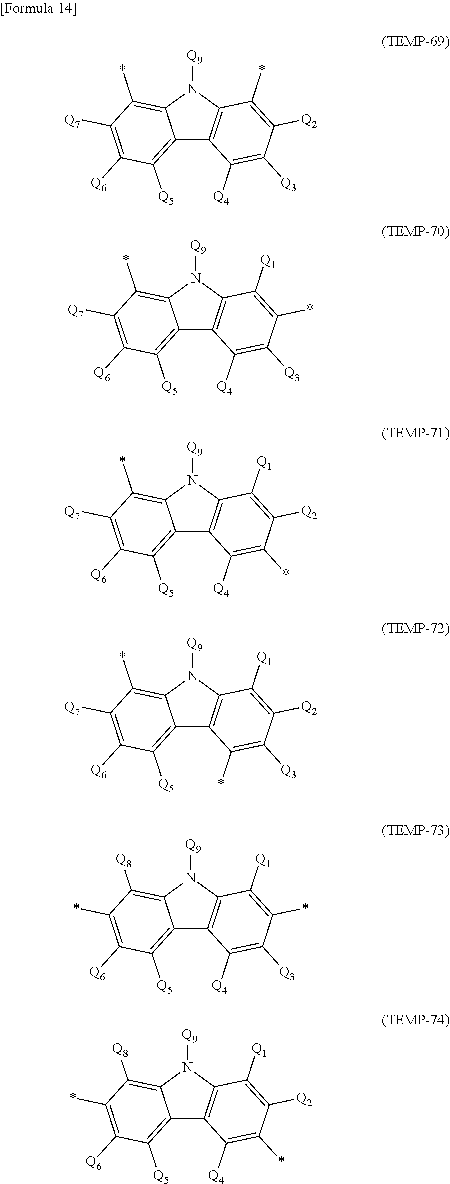

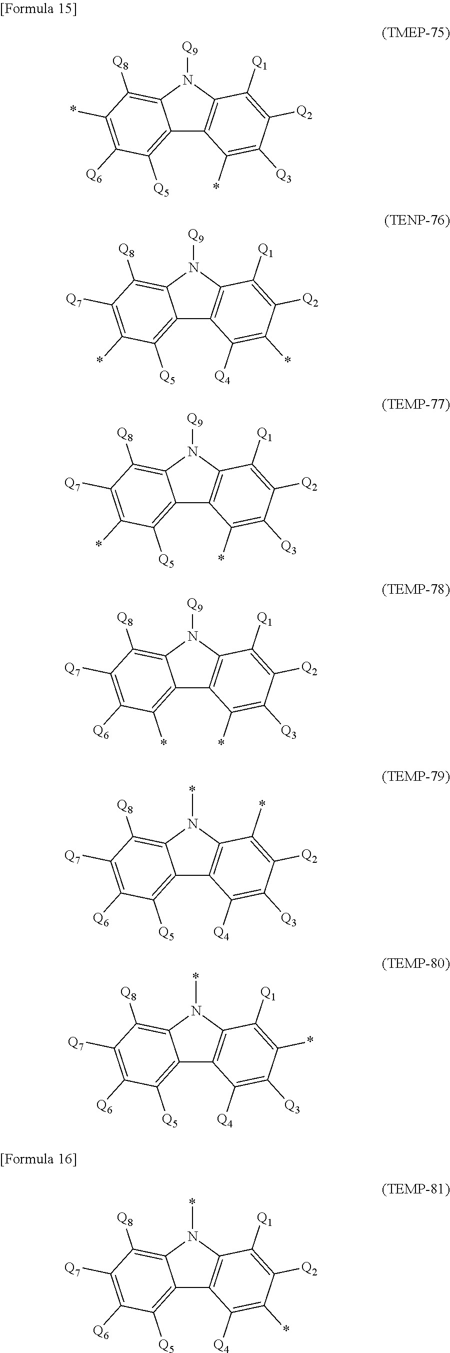

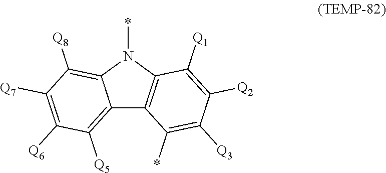

In the formulae (TEMP-69) to (TEMP-82), Q₁ to Q₉ are each independently a hydrogen atom or a substituent.

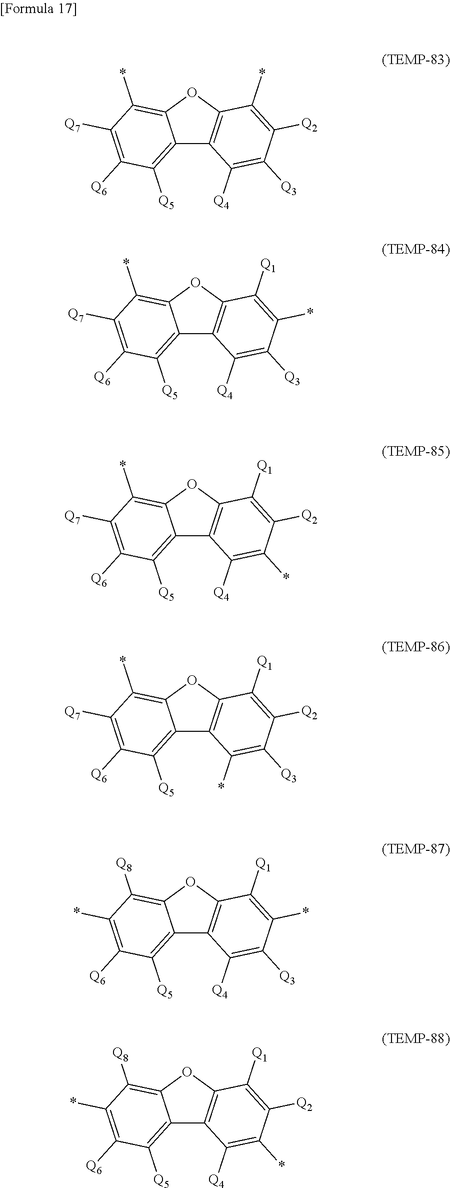

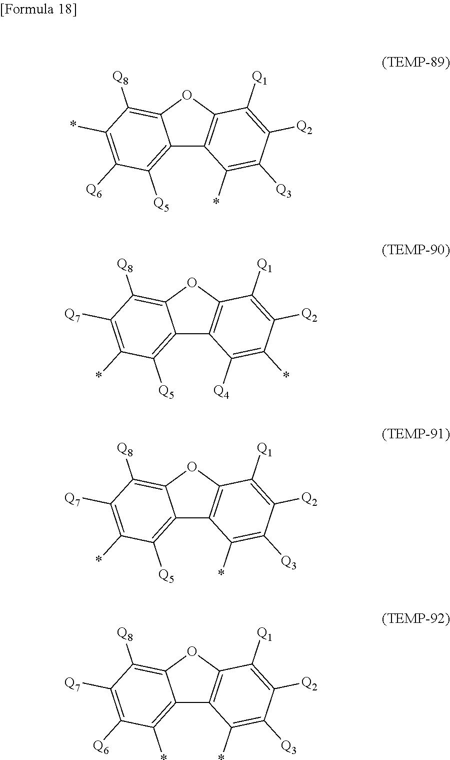

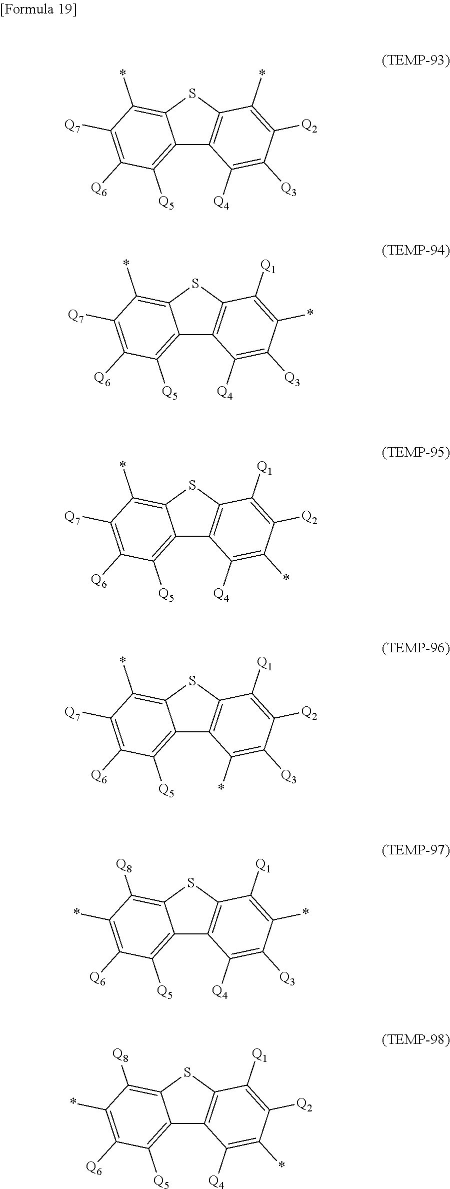

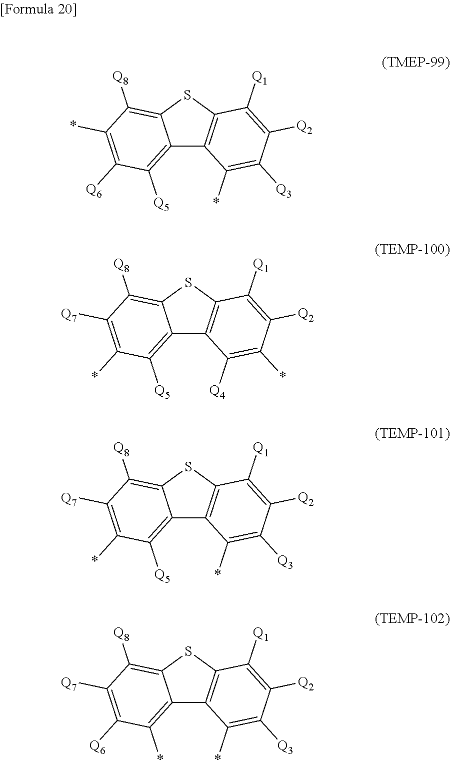

In the formulae (TEMP-83) to (TEMP-102), Q₁ to Q₈ are each independently a hydrogen atom or a substituent.

The substituent mentioned herein has been described above.

### Instance of “Bonded to Form Ring”

Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring, “at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (these instances will be sometimes collectively referred to as an instance of “bonded to form a ring” hereinafter) will be described below. An anthracene compound having a basic skeleton in a form of an anthracene ring and represented by a formula (TEMP-103) below will be used as an example for the description.

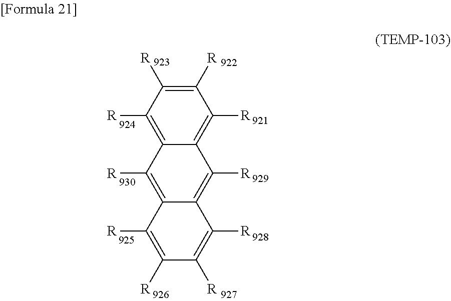

For instance, when “at least one combination of adjacent two or more of R₉₂₁ to R₉₃₀ are mutually bonded to form a ring,” the combination of adjacent ones of R₉₂₁ to R₉₃₀ (i.e. the combination at issue) is a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, or a combination of R₉₂₉ and R₉₂₁.

The term “at least one combination” means that two or more of the above combinations of adjacent two or more of R₉₂₁ to R₉₃₀ may simultaneously form rings. For instance, when R₉₂₁ and R₉₂₂ are mutually bonded to form a ring Q_{A} and R₉₂₅ and R₉₂₆ are simultaneously mutually bonded to form a ring Q_{B}, the anthracene compound represented by the formula (TEMP-103) is represented by a formula (TEMP-104) below.

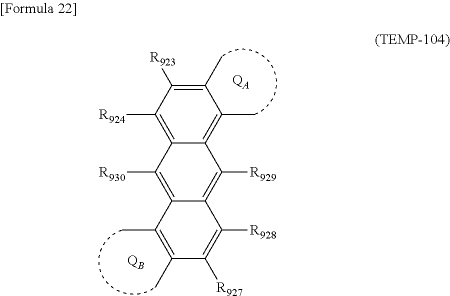

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring Q_{A} and R₉₂₂ and R₉₂₃ are mutually bonded to form a ring Qc, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring Q_{A} and the ring Qc share R₉₂₂.

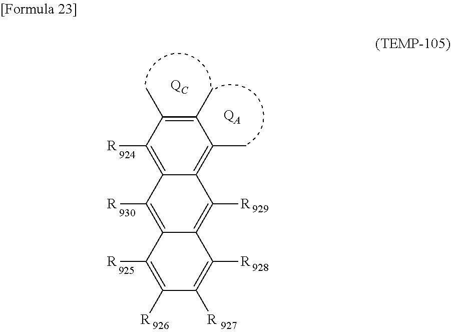

The formed “monocyclic ring” or “fused ring” may be, in terms of the formed ring in itself, a saturated ring or an unsaturated ring. When the “combination of adjacent two” form a “monocyclic ring” or a “fused ring,” the “monocyclic ring” or “fused ring” may be a saturated ring or an unsaturated ring. For instance, the ring Q_{A} and the ring Q_{B} formed in the formula (TEMP-104) are each independently a “monocyclic ring” or a “fused ring.” Further, the ring Q_{A} and the ring Qc formed in the formula (TEMP-105) are each a “fused ring.” The ring Q_{A} and the ring Qc in the formula (TEMP-105) are fused to form a fused ring. When the ring Q_{A} in the formula (TMEP-104) is a benzene ring, the ring Q_{A} is a monocyclic ring. When the ring Q_{A} in the formula (TMEP-104) is a naphthalene ring, the ring Q_{A} is a fused ring.

The “unsaturated ring” represents an aromatic hydrocarbon ring or an aromatic heterocycle. The “saturated ring” represents an aliphatic hydrocarbon ring or a non-aromatic heterocycle.

Specific examples of the aromatic hydrocarbon ring include a ring formed by terminating a bond of a group in the specific example of the specific example group G1 with a hydrogen atom.

Specific examples of the aromatic heterocycle include a ring formed by terminating a bond of an aromatic heterocyclic group in the specific example of the specific example group G2 with a hydrogen atom.

Specific examples of the aliphatic hydrocarbon ring include a ring formed by terminating a bond of a group in the specific example of the specific example group G6 with a hydrogen atom.

The phrase “to form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded to R₉₂₁, a carbon atom of the anthracene skeleton bonded to R₉₂₂, and one or more optional atoms. Specifically, when the ring Q_{A} is a monocyclic unsaturated ring formed by R₉₂₁ and R₉₂₂, the ring formed by a carbon atom of the anthracene skeleton bonded to R₉₂₁, a carbon atom of the anthracene skeleton bonded to R₉₂, and four carbon atoms is a benzene ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes an optional element other than carbon atom, the resultant ring is a heterocycle.

The number of “one or more optional atoms” forming the monocyclic ring or fused ring is, unless otherwise specified herein, preferably in a range from 2 to 15, more preferably in a range from 3 to 12, further preferably in a range from 3 to 5.

Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

When the “saturated ring” or the “unsaturated ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance of “bonded to form a ring”).

### Substituent for Substituted or Unsubstituted Group

In an exemplary embodiment herein, a substituent for the substituted or unsubstituted group (sometimes referred to as an “optional substituent” hereinafter) is, for instance, a group selected from the group consisting of an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms.

R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

when two or more R₉₀₁ are present, the two or more R₉₀₁ are mutually the same or different;

when two or more R₉₀₂ are present, the two or more R₉₀₂ are mutually the same or different;

when two or more R₉₀₃ are present, the two or more R₉₀₃ are mutually the same or different;

when two or more R₉₀₄ are present, the two or more R₉₀₄ are mutually the same or different;

when two or more R₉₀₅ are present, the two or more R₉₀₅ are mutually the same or different;

when two or more R₉₀₆ are present, the two or more R₉₀₆ are mutually the same or different; and

when two or more R₉₀₇ are present, the two or more R₉₀₇ are mutually the same or different.

In an exemplary embodiment, a substituent for the substituted or unsubstituted group is selected from the group consisting of an alkyl group having 1 to 50 carbon atoms, an aryl group having 6 to 50 ring carbon atoms, and a heterocyclic group having 5 to 50 ring atoms.

In an exemplary embodiment, a substituent for the substituted or unsubstituted group is selected from the group consisting of an alkyl group having 1 to 18 carbon atoms, an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted unsaturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

Herein, numerical ranges represented by “AA to BB” represent a range whose lower limit is the value (AA) recited before “to” and whose upper limit is the value (BB) recited after “to.”

### First Exemplary Embodiment

### Organic Electroluminescence Device

An organic electroluminescence device according to a first exemplary embodiment includes an emitting layer between an anode and a cathode, and a first layer between the cathode and the emitting layer, in which the first layer has a thickness of 50 nm or more, the first layer contains a compound represented by a formula (100) below, and the first layer contains no metal doping material.

Herein, the metal doping material is a metal, metal compound or metal complex with a work function of 4.2 eV or less. The metal, metal compound or metal complex with a work function of 4.2 eV or less is a metal, metal compound or metal complex selected from the group consisting of an alkali metal, an alkaline earth metal, a transition metal including a rare earth metal, a compound including the alkali metal, a compound including the alkaline earth metal, a compound including the transition metal, a complex including the alkali metal, a complex including the alkaline earth metal, and a complex including the transition metal. Examples of the metal doping material include metals such as lithium (Li), sodium (Na), potassium (K), rubidium (Rb), cesium (Cs), calcium (Ca), strontium (Sr) and barium (Ba), metal compounds such as cesium carbonate, and metal complexes such as Liq.

The organic EL device according to the exemplary embodiment may include one or more organic layer(s) in addition to the emitting layer and the first layer. Examples of the organic layer include, for instance, at least one layer selected from the group consisting of a hole injecting layer, a hole transporting layer, an emitting layer, an electron injecting layer, an electron transporting layer, a hole blocking layer, and an electron blocking layer.

In the organic EL device according to the exemplary embodiment, the organic layer may consist of the emitting layer and the first layer. Alternatively, the organic layer may further include, for instance, at least one layer selected from the group consisting of the hole injecting layer, the hole transporting layer, the electron injecting layer, the electron transporting layer, the hole blocking layer, and the electron blocking layer.

### Schematic Structure of Organic EL Device

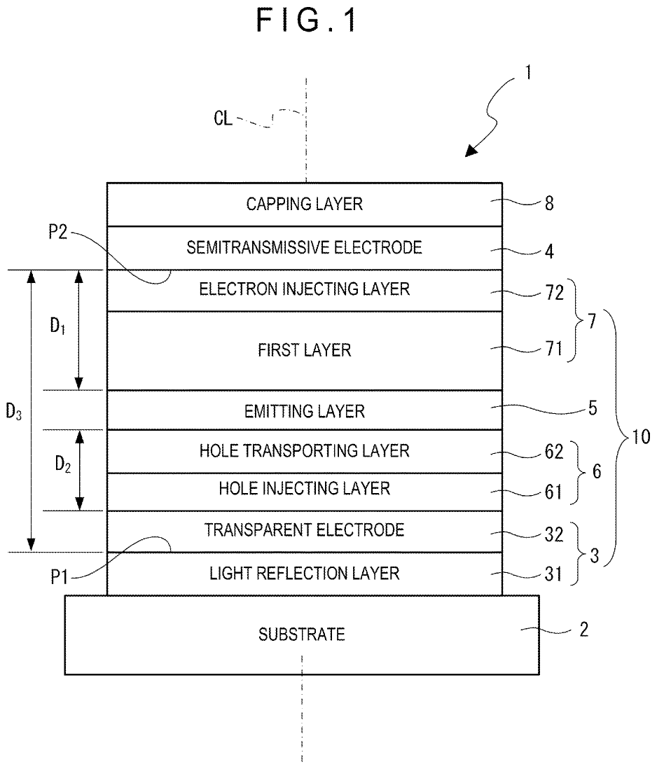

FIG. 1 schematically shows an exemplary arrangement of the organic EL device according to the exemplary embodiment.

An organic EL device 1 includes a substrate 2, an anode 3, a semitransmissive electrode 4 as a cathode, and an organic layer 10 provided between the anode 3 and the semitransmissive electrode 4. The organic EL device 1 includes a capping layer 8 provided on a side opposite to the organic layer with respect to the semitransmissive electrode 4.

The organic layer 10 includes a hole transporting zone 6, an emitting layer 5 and an electron transporting zone 7, which are sequentially laminated on the anode 3.

In the organic EL device according to the exemplary embodiment, the anode 3 includes a light reflection layer 31 and a transparent electrode 32. The light reflection layer 31 and the transparent electrode 32 of the anode 3 are sequentially laminated on the substrate 2.

In the organic EL device according to the exemplary embodiment, the hole transporting zone 6 includes a hole injecting layer 61 and a hole transporting layer 62. The hole injecting layer 61 and the hole transporting layer 62 of the hole transporting zone 6 are sequentially laminated on the transparent electrode 32.

In the organic EL device according to the exemplary embodiment, the electron transporting zone 7 includes a first layer 71 and an electron injecting layer 72. The first layer 71 and the electron injecting layer 72 of the electron transporting zone 7 are sequentially laminated on the emitting layer 5.

In the organic EL device according to the exemplary embodiment, it is also preferable that the emitting layer and the first layer are in direct contact with each other.

In the example of the organic EL device according to the exemplary embodiment shown in FIG. 1, the emitting layer 5 and the first layer 71 are in direct contact with each other.

It is also preferable that the organic EL device according to the exemplary embodiment further includes a second layer between the emitting layer and the first layer.

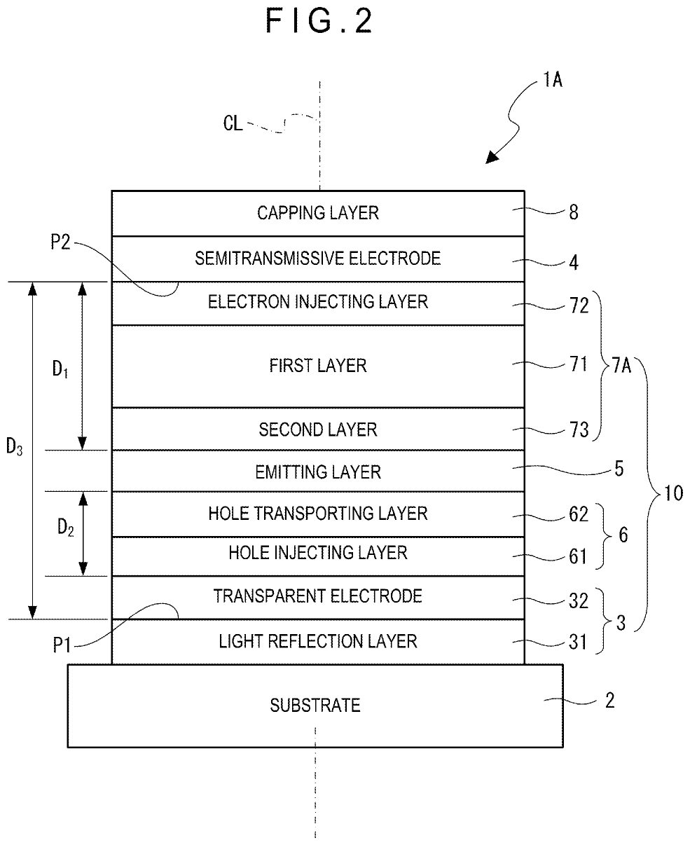

FIG. 2 schematically shows another exemplary arrangement of the organic EL device according to the exemplary embodiment.

An organic EL device 1A includes a substrate 2, an anode 3, a semitransmissive electrode 4 as a cathode, and an organic layer 10 provided between the anode 3 and the semitransmissive electrode 4. The organic EL device 1A includes a capping layer 8 provided on a side opposite to the organic layer with respect to the semitransmissive electrode 4.

Also in the organic EL device 1A, the organic layer 10 includes a hole transporting zone 6, an emitting layer 5 and an electron transporting zone 7A, which are sequentially laminated on the anode 3.

Also in the organic EL device 1A, the anode 3 includes a light reflection layer 31 and a transparent electrode 32. The light reflection layer 31 and the transparent electrode 32 of the anode 3 are sequentially laminated on the substrate 2.

Also in the organic EL device 1A, the hole transporting zone 6 includes a hole injecting layer 61 and a hole transporting layer 62. The hole injecting layer 61 and the hole transporting layer 62 of the hole transporting zone 6 are sequentially laminated on the transparent electrode 32.

In the organic EL device 1A, the electron transporting zone 7A includes a first layer 71, an electron injecting layer 72 and a second layer 73. The second layer 73, the first layer 71 and the electron injecting layer 72 of the electron transporting zone 7A are sequentially laminated on the emitting layer 5.

It is also preferable that the organic EL device according to the exemplary embodiment further includes a third layer between the cathode and the first layer.

In the organic EL device according to the exemplary embodiment, the third layer is preferably an organic compound layer containing an alkali metal, an alkaline earth metal, a compound of an alkali metal or a compound of an alkaline earth metal.

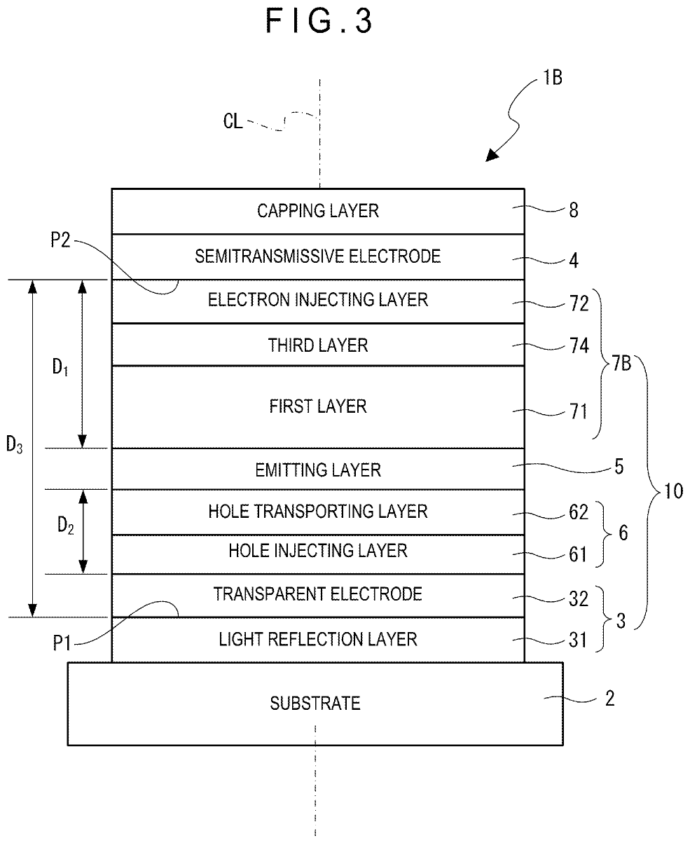

FIG. 3 schematically shows still another exemplary arrangement of the organic EL device according to the exemplary embodiment.

An organic EL device 1B includes a substrate 2, an anode 3, a semitransmissive electrode 4 as a cathode, and an organic layer 10 provided between the anode 3 and the semitransmissive electrode 4. The organic EL device 1B includes a capping layer 8 provided on a side opposite to the organic layer with respect to the semitransmissive electrode 4.

Also in the organic EL device 1B, the organic layer 10 includes a hole transporting zone 6, an emitting layer 5 and an electron transporting zone 7B, which are sequentially laminated on the anode 3.

The anode 3 and the hole transporting zone 6 in the organic EL device 1B are configured in the same manner as those in the organic EL device 1 or the organic EL device 1A.

In the organic EL device 1B, the electron transporting zone 7B includes a first layer 71, a third layer 74 and an electron injecting layer 72. The first layer 71, the third layer 74 and the electron injecting layer 72 of the electron transporting zone 7B are sequentially laminated on the emitting layer 5.

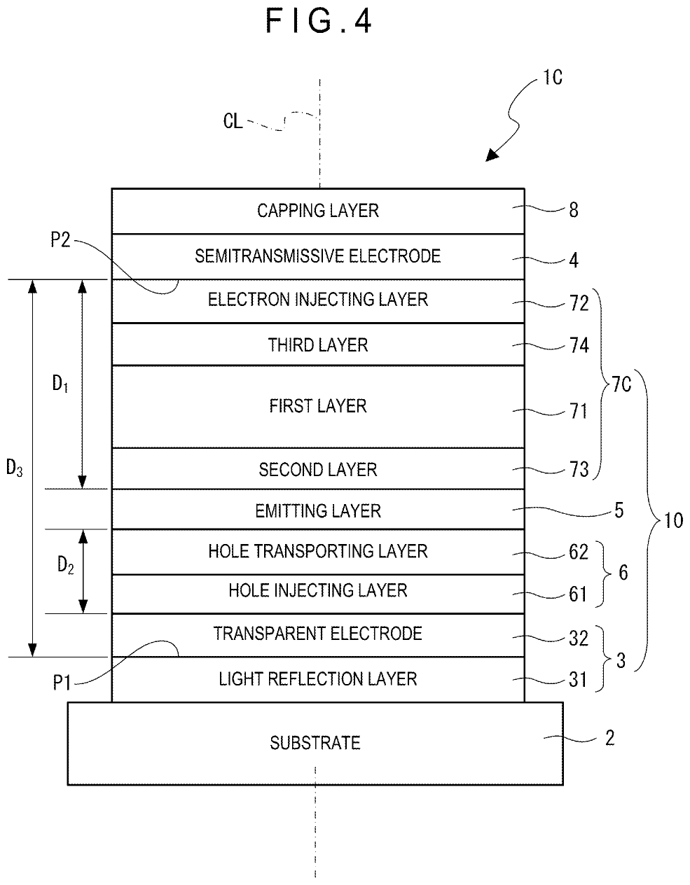

FIG. 4 schematically shows a further exemplary arrangement of the organic EL device according to the exemplary embodiment.

An organic EL device 1C includes a substrate 2, an anode 3, a semitransmissive electrode 4 as a cathode, and an organic layer 10 provided between the anode 3 and the semitransmissive electrode 4. The organic EL device 1C includes a capping layer 8 provided on a side opposite to the organic layer with respect to the semitransmissive electrode 4.

Also in the organic EL device 1C, the organic layer 10 includes a hole transporting zone 6, an emitting layer 5 and an electron transporting zone 7C, which are sequentially laminated on the anode 3.

The anode 3 and the hole transporting zone 6 in the organic EL device 1C are configured in the same manner as those in the organic EL device 1 or the organic EL device 1A.

In the organic EL device 1C, the electron transporting zone 7C includes a first layer 71, a second layer 73, a third layer 74 and an electron injecting layer 72. The second layer 73, the first layer 71, the third layer 74 and the electron injecting layer 72 of the electron transporting zone 7C are sequentially laminated on the emitting layer 5.

In the organic EL device according to the exemplary embodiment, it is preferable that a distance D₁ between an interface of the cathode close to the emitting layer and an interface of the emitting layer close to the cathode is larger than a distance D₂ between an interface of the anode close to the emitting layer and an interface of the emitting layer close to the anode.

Also in the organic EL device 1, the organic EL device 1A, the organic EL device 1B or the organic EL device 1C, it is preferable that a distance D₁ between an interface of the semitransmissive electrode 4 as the cathode close to the emitting layer 5 and an interface of the emitting layer 5 close to the semitransmissive electrode 4 as the cathode is larger than a distance D₂ between an interface of the anode 3 close to the emitting layer 5 and an interface of the emitting layer 5 close to the anode 3.

### First Layer

The first layer is a layer provided between the cathode and the emitting layer.

For instance, in the organic EL device 1, the first layer 71 is a layer provided between the emitting layer 5 and the electron injecting layer 72. In the organic EL device 1A, the first layer 71 is a layer provided between the second layer 73 and the electron injecting layer 72. In the organic EL device 1B, the first layer 71 is a layer provided between the emitting layer and the third layer 74. In the organic EL device 1C, the first layer 71 is a layer provided between the second layer 73 and the third layer 74.

The thickness of the first layer is 50 nm or more, preferably 70 nm or more, more preferably 100 nm or more, further preferably 120 nm or more, in terms of conditions for optical interference.

The thickness of the first layer is preferably 160 nm or less, more preferably 150 nm or less.

The thickness of the first layer is preferably larger than a thickness of a layer other than the first layer that is provided between the cathode and the emitting layer. For instance, in the organic EL device 1 shown in FIG. 1, the thickness of the first layer 71 is preferably larger than the thickness of the electron injecting layer 72. Further, in the organic EL device 1A shown in FIG. 2, the thickness of the first layer 71 is preferably larger than the thickness of the second layer 73 and the thickness of the electron injecting layer 72.

### Compound Represented by Formula (100)

The first layer of the organic EL device according to the exemplary embodiment contains a compound represented by a formula (100) below.

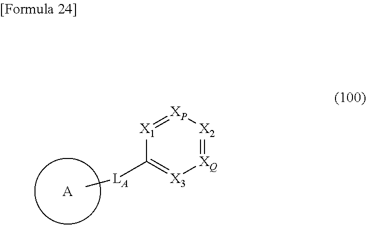

In the formula (100):

A is a substituted or unsubstituted fused aryl group having 13 to 50 ring carbon atoms, or a substituted or unsubstituted fused heterocyclic group having 14 to 50 ring atoms;

L_{A} is a single bond, a substituted or unsubstituted arylene group having 6 to 30 ring carbon atoms, or a substituted or unsubstituted divalent heterocyclic group having 5 to 30 ring atoms;

X₁, X₂ and X₃ are each independently a nitrogen atom or CR₃;

X_{P} is a nitrogen atom or CR₁;

X_{Q} is a nitrogen atom or CR₂;

at least one of X₁, X₂, X₃, X_{P} or X_{Q} is a nitrogen atom;

at least one combination of adjacent two or more of R₁, R₂ and R₃ are mutually bonded to form a substituted or unsubstituted monocyclic ring, mutually bonded to form a substituted or unsubstituted fused ring, or not mutually bonded;

R₁, R₂ and R₃ not forming the substituted or unsubstituted monocyclic ring and not forming the substituted or unsubstituted fused ring are each independently a hydrogen atom, a substituted or unsubstituted aryl group having 6 to 30 ring carbon atoms, or a substituted or unsubstituted heterocyclic group having 5 to 30 ring atoms; and

when a plurality of R₃ are present, the plurality of R₃ are mutually the same or different.

In the formula (100), it is also preferable that X_{P} is CR₁ and X_{Q} is CR₂. In this case, the compound represented by the formula (100) is a compound represented by a formula (1) below.

### Compound Represented by Formula (1)

The compound represented by the formula (100) is also preferably a compound represented by the formula (1).

The first layer of the organic EL device according to the exemplary embodiment also preferably contains a compound represented by the formula (1).

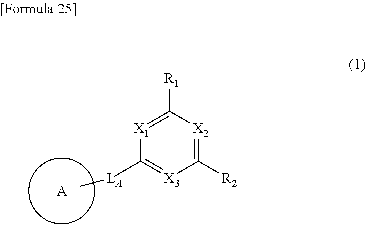

In the formula (1):

A is a substituted or unsubstituted fused aryl group having 13 to 50 ring carbon atoms, or a substituted or unsubstituted fused heterocyclic group having 14 to 50 ring atoms;

L_{A} is a single bond, a substituted or unsubstituted arylene group having 6 to 30 ring carbon atoms, or a substituted or unsubstituted divalent heterocyclic group having 5 to 30 ring atoms;

X₁, X₂ and X₃ are each independently a nitrogen atom or CR₃;

at least one of X₁, X₂ or X₃ is a nitrogen atom;

at least one combination of adjacent two or more of R₁, R₂ and R₃ are mutually bonded to form a substituted or unsubstituted monocyclic ring, mutually bonded to form a substituted or unsubstituted fused ring, or not mutually bonded;

R₁, R₂ and R₃ not forming the substituted or unsubstituted monocyclic ring and not forming the substituted or unsubstituted fused ring are each independently a hydrogen atom, a substituted or unsubstituted aryl group having 6 to 30 ring carbon atoms, or a substituted or unsubstituted heterocyclic group having 5 to 30 ring atoms; and

when a plurality of R₃ are present, the plurality of R₃ are mutually the same or different.

Herein, the fused aryl group is a monovalent aryl group derived by removing one hydrogen atom from a cyclic structure in which a plurality of aromatic hydrocarbon rings as monocyclic rings are fused to each other. Examples of the fused aryl group include a 1-naphthyl group, 2-naphthyl group, anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, perylenyl group, and a monovalent aryl group derived by removing one hydrogen atom from any of cyclic structures represented by the formulae (TEMP-1) to (TEMP-15). Examples of the fused aryl group herein does not include a group in which a plurality of monocyclic rings are linked by a single bond (e.g., a biphenyl group and a terphenyl group).

In the organic EL device according to the exemplary embodiment, the fused aryl group having 13 to 50 ring carbon atoms is a group selected from the above fused aryl groups and having 13 to 50 ring carbon atoms.

Herein, the fused heterocyclic group is a monovalent heterocyclic group derived by removing one hydrogen atom from a cyclic structure in which at least one heterocycle as a monocyclic ring is fused to at least one ring selected from the group consisting of a heterocycle as a monocyclic ring and an aromatic hydrocarbon ring as a monocyclic ring. Examples of the fused heterocyclic group include an indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, diazacarbazolyl group, xanthenyl group, benzofuranyl group, isobenzofuranyl group, dibenzofuranyl group, naphthobenzofuranyl group, benzoxazolyl group, benzisoxazolyl group, phenoxazinyl group, dinaphthofuranyl group, azadibenzofuranyl group, diazadibenzofuranyl group, azanaphthobenzofuranyl group, diazanaphthobenzofuranyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), diazanaphthobenzothiophenyl group (diazanaphthobenzothienyl group), and a monovalent heterocyclic group derived by removing at least one hydrogen atom from any of cyclic structures represented by the formulae (TEMP-16) to (TEMP-33).

In the organic EL device according to the exemplary embodiment, the fused heterocyclic group having 14 to 50 ring atoms is a group selected from the above fused heterocyclic groups and having 14 to 50 ring atoms.

In the organic EL device according to the exemplary embodiment, the compound represented by the formula (100) is also preferably a compound represented by a formula (101) below.

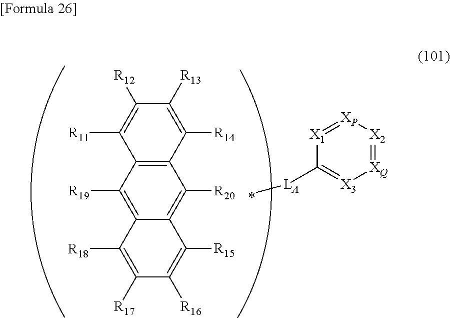

In the formula (101):

X₁ to X₃, X_{P}, X_{Q}, R₁ to R₃ and L_{A} each represent the same as defined in the formula (100);

one of R₁₁ to R₂₀ is a bonding position * to L_{A};

at least one combination of adjacent two or more of R₁₁ to R₂₀ not being the bonding position to L_{A} are mutually bonded to form a substituted or unsubstituted monocyclic ring, mutually bonded to form a substituted or unsubstituted fused ring, or not mutually bonded; and

R₁₁ to R₂₀ not being the bonding position to L_{A}, not forming the substituted or unsubstituted monocyclic ring and not forming the substituted or unsubstituted fused ring each independently represent the same as defined in a formula (A1) below.

In the organic EL device according to the exemplary embodiment, the compound represented by the formula (1) is also preferably a compound represented by the formula (A1).

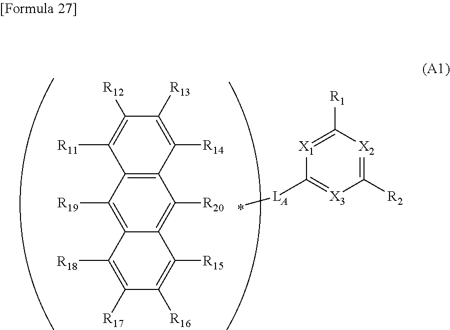

In the formula (A1):

X₁ to X₃, R₁ to R₃ and L_{A} each represent the same as defined in the formula (1);

one of R₁₁ to R₂₀ is a bonding position * to L_{A};

at least one combination of adjacent two or more of R₁₁ to R₂₀ not being the bonding position to L_{A} are mutually bonded to form a substituted or unsubstituted monocyclic ring, mutually bonded to form a substituted or unsubstituted fused ring, or not mutually bonded;

R₁₁ to R₂₀ not being the bonding position to L_{A}, not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(Re)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;

R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

when a plurality of R₉₀₁ are present, the plurality of R₉₀₁ are mutually the same or different;

when a plurality of R₉₀₂ are present, the plurality of R₉₀₂ are mutually the same or different;

when a plurality of R₉₀₃ are present, the plurality of R₉₀₃ are mutually the same or different;

when a plurality of R₉₀₄ are present, the plurality of R₉₀₄ are mutually the same or different;

when a plurality of R₉₀₅ are present, the plurality of R₉₀₅ are mutually the same or different;

when a plurality of R₉₀₆ are present, the plurality of R₉₀₆ are mutually the same or different;

when a plurality of R₉₀₇ are present, the plurality of R₉₀₇ are mutually the same or different;

when a plurality of R₈₀₁ are present, the plurality of R₈₀₁ are mutually the same or different; and

when a plurality of R₈₀₂ are present, the plurality of R₈₀₂ are mutually the same or different.

In the organic EL device according to the exemplary embodiment, when a combination of adjacent R₁₂ and R₁₃ are mutually bonded to form a substituted or unsubstituted monocyclic ring or mutually bonded to form a substituted or unsubstituted fused ring, the compound represented by the formula (1) is represented by a formula (A-Q1) below.

In the organic EL device according to the exemplary embodiment, when a combination of adjacent R₁₃ and R₁₄ are mutually bonded to form a substituted or unsubstituted monocyclic ring or mutually bonded to form a substituted or unsubstituted fused ring, the compound represented by the formula (1) is represented by a formula (A-Q2) below.

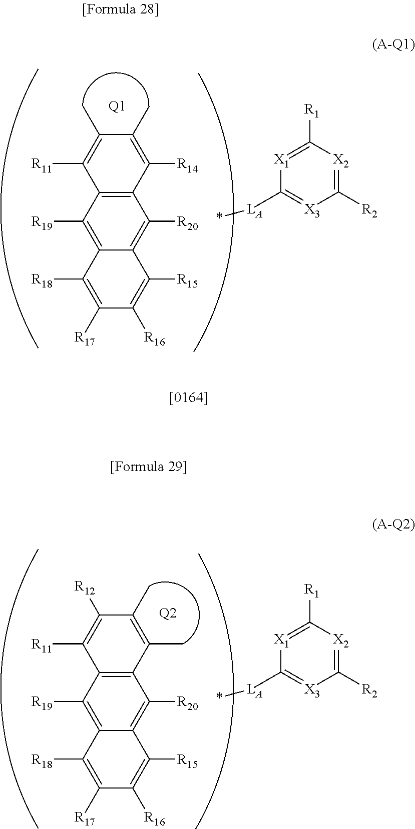

In the formulae (A-Q1) and (A-Q2):

a ring Q1 and a ring Q2 are each independently a substituted or unsubstituted monocyclic ring or a substituted or unsubstituted fused ring;

X₁ to X₃, R₁ to R₃ and L_{A} each represent the same as defined in the formula (1); and

R₁₁ to R₂₀ not being the bonding position to L_{A}, not forming the substituted or unsubstituted monocyclic ring and not forming the substituted or unsubstituted fused ring each independently represent the same as defined in the formula (A1).

It is preferable that the ring Q1 and the ring Q2 are each independently a substituted or unsubstituted aromatic hydrocarbon ring or a substituted or unsubstituted aromatic heterocycle.

In the organic EL device according to the exemplary embodiment, it is preferable that two or more of R₁₁ to R₂₀ not being the bonding position to L_{A}, not forming the substituted or unsubstituted monocyclic ring and not forming the substituted or unsubstituted fused ring are each independently not a hydrogen atom.

In the organic EL device according to the exemplary embodiment, it is preferable that two or more of R₁₁ to R₂₀ not being the bonding position to L_{A}, not forming the substituted or unsubstituted monocyclic ring and not forming the substituted or unsubstituted fused ring are each independently a substituted or unsubstituted aryl group having 6 to 50 ring carbon atoms, or a substituted or unsubstituted heterocyclic group having 5 to 50 ring atoms.

In the organic EL device according to the exemplary embodiment, it is preferable that R₁₉ and R₂₀ are each independently a substituted or unsubstituted aryl group having 6 to 50 ring carbon atoms, or a substituted or unsubstituted heterocyclic group having 5 to 50 ring atoms.

In the organic EL device according to the exemplary embodiment, it is also preferable that R₁₂, R₁₃, R₁₆ or R₁₇ is the bonding position to L_{A}. When R₁₃ is the bonding position to L_{A}, the compound represented by the formula (100) is represented by a formula (102) below and the compound represented by the formula (1) is represented by a formula (A1-1) below.

In the organic EL device according to the exemplary embodiment, the compound represented by the formula (100) is also preferably a compound represented by the formula (102).

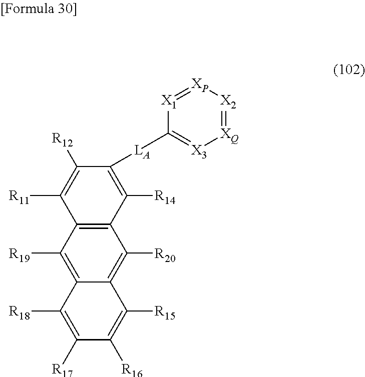

In the formula (102):

X₁ to X₃, X_{P}, X_{Q}, R₁ to R₃ and L_{A} each represent the same as defined in the formula (100); and

R₁₁, R₁₂ and R₁₄ to R₂₀ each independently represent the same as defined in the formula (A1-1).

In the compound represented by the formula (100), it is also preferable that at least one of X_{P} or X_{Q} is a nitrogen atom.

In the compound represented by the formula (100), it is also preferable that X_{P} is CR₁ and X_{Q} is a nitrogen atom.

In the compound represented by the formula (100), it is also preferable that X_{P} is CR₁, X_{Q} is a nitrogen atom, and X₁, X₂ and X₃ are each CR₃.

In the compound represented by the formula (100), it is also preferable that X_{P} is a nitrogen atom and X_{Q} is CR₂.

In the compound represented by the formula (100), it is also preferable that X_{P} is a nitrogen atom, X_{Q} is CR₂, and X₁, X₂ and X₃ are each CR₃.

In the organic EL device according to the exemplary embodiment, the compound represented by the formula (1) is also preferably a compound represented by the formula (A1-1).

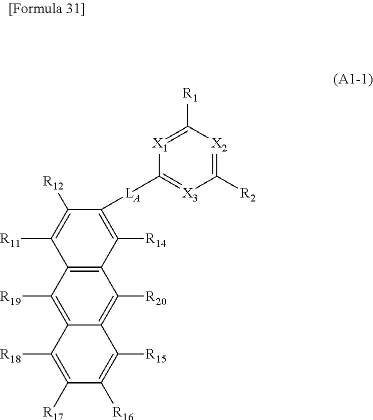

In the formula (A1-1):

X₁ to X₃, R₁ to R₃ and L_{A} each represent the same as defined in the formula (1);

at least one combination of adjacent two or more of R₁₁, R₁₂ and R₁₄ to R₂₀ are mutually bonded to form a substituted or unsubstituted monocyclic ring, mutually bonded to form a substituted or unsubstituted fused ring, or not mutually bonded;

R₁₁, R₁₂ and R₁₄ to R₂₀ not being the bonding position to L_{A}, not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and

R₉₀₁, R₉₀₂, R₉₀₃, R₉₀₄, R₉₀₅, R₉₀₆, R₉₀₇, R₈₀₁ and R₈₀₂ each represent the same as defined in the formula (A1).

In the formula (A1-1), it is preferable that R₁₉ and R₂₀ are each independently a substituted or unsubstituted aryl group having 6 to 50 ring carbon atoms, or a substituted or unsubstituted heterocyclic group having 5 to 50 ring atoms.

In the organic EL device according to the exemplary embodiment, it is also preferable that R₁₉ or R₂₀ is the bonding position to L_{A}. When R₂₀ is the bonding position to L_{A}, the compound represented by the formula (1) is represented by a formula (A1-2) below.

In the organic EL device according to the exemplary embodiment, the compound represented by the formula (1) is also preferably a compound represented by the formula (A1-2).

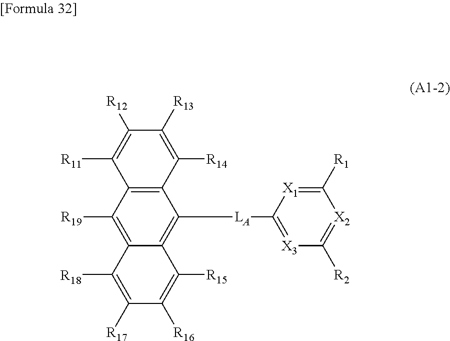

In the formula (A1-2):

X₁ to X₃, R₁ to R₃ and L_{A} each represent the same as defined in the formula (1);

at least one combination of adjacent two or more of R₁₁ to R₁₉ are mutually bonded to form a substituted or unsubstituted monocyclic ring, mutually bonded to form a substituted or unsubstituted fused ring, or not mutually bonded;

R₁₁ to R₁₉ not being the bonding position to L_{A}, not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and

R₉₀₁, R₉₀₂, R₉₀₃, R₉₀₄, R₉₀₅, R₉₀₆, R₉₀₇, R₈₀₁ and R₈₀₂ each represent the same as defined in the formula (A1).

In the organic EL device according to the exemplary embodiment, it is preferable that R₁₉ is a substituted or unsubstituted aryl group having 6 to 50 ring carbon atoms, or a substituted or unsubstituted heterocyclic group having 5 to 50 ring atoms.

In the organic EL device according to the exemplary embodiment, it is preferable that R₁₉ is a substituted or unsubstituted aryl group having 6 to 50 ring carbon atoms, or a substituted or unsubstituted heterocyclic group having 5 to 50 ring atoms, and

R₁ and R₂ are each independently a substituted or unsubstituted aryl group having 6 to 50 ring carbon atoms.

In the organic EL device according to the exemplary embodiment, the compound represented by the formula (1) is also preferably a compound represented by a formula (B1) below.

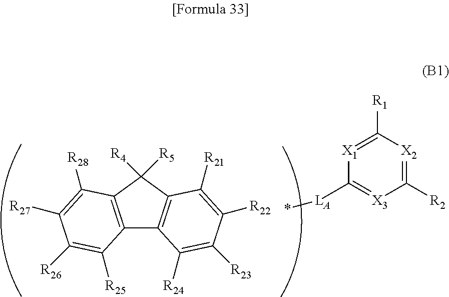

In the formula (B1):

X₁ to X₃, R₁ to R₃ and L_{A} each represent the same as defined in the formula (1);

one of R₂₁ to R₂₈ is a bonding position * to L_{A};

R₂₁ to R₂₈ not being the bonding position to L_{A} are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;

a combination of R₄ and R₅ are mutually bonded to form a substituted or unsubstituted monocyclic ring, mutually bonded to form a substituted or unsubstituted fused ring, or not mutually bonded;

R₄ and R₅ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms;

R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

when a plurality of R₉₀₁ are present, the plurality of R₉₀₁ are mutually the same or different;

when a plurality of R₉₀₂ are present, the plurality of R₉₀₂ are mutually the same or different;

when a plurality of R₉₀₃ are present, the plurality of R₉₀₃ are mutually the same or different;

when a plurality of R₉₀₄ are present, the plurality of R₉₀₄ are mutually the same or different;

when a plurality of R₉₀₅ are present, the plurality of R₉₀₅ are mutually the same or different;

when a plurality of R₉₀₆ are present, the plurality of R₉₀₆ are mutually the same or different;

when a plurality of R₉₀₇ are present, the plurality of R₉₀₇ are mutually the same or different;

when a plurality of R₈₀₁ are present, the plurality of R₈₀₁ are mutually the same or different; and

when a plurality of R₈₀₂ are present, the plurality of R₈₀₂ are mutually the same or different.

In the organic EL device according to the exemplary embodiment, it is preferable that R₄ and R₅ are each independently a substituted or unsubstituted aryl group having 6 to 50 ring carbon atoms.

In the organic EL device according to the exemplary embodiment, it is preferable that R₄ and R₅ are each independently a substituted or unsubstituted phenyl group.

In the organic EL device according to the exemplary embodiment, the compound represented by the formula (B1) is preferably a compound represented by a formula (B1-1) below.

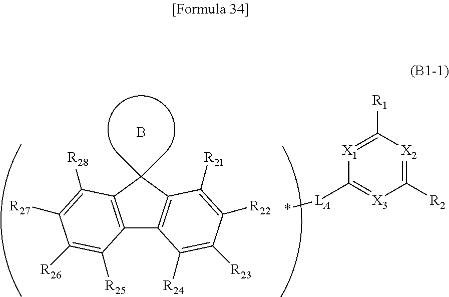

In the formula (B1-1):

X₁ to X₃, R₁ to R₃ and L_{A} each represent the same as defined in the formula (1);

R₂₁ to R₂₈ each represent the same as defined in the formula (B1); and

a ring B is a substituted or unsubstituted monocyclic ring or a substituted or unsubstituted fused ring.

It is preferable that the ring B is a substituted or unsubstituted aromatic hydrocarbon ring or a substituted or unsubstituted aromatic heterocycle.

In the organic EL device according to the exemplary embodiment, the compound represented by the formula (B1) is preferably a compound represented by a formula (B1-1A) below.

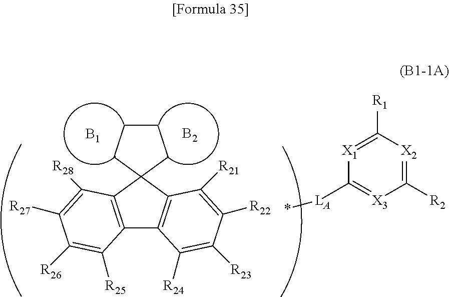

In the formula (B1-1A):

X₁ to X₃, R₁ to R₃ and L_{A} each represent the same as defined in the formula (1);

R₂₁ to R₂₈ each represent the same as defined in the formula (B1); and

a ring B₁ and a ring B₂ are each independently a substituted or unsubstituted monocyclic ring or a substituted or unsubstituted fused ring.

It is preferable that the ring B₁ and the ring B₂ are each independently a substituted or unsubstituted aromatic hydrocarbon ring or a substituted or unsubstituted aromatic heterocycle.

In the organic EL device according to the exemplary embodiment, the compound represented by the formula (B1) is preferably a compound represented by a formula (B1-2) below.

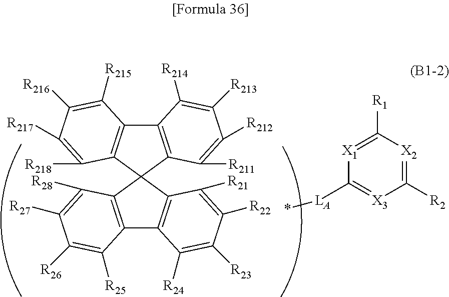

In the formula (B1-2):

X₁ to X₃, R₁ to R₃ and L_{A} each represent the same as defined in the formula (1);

R₂₁ to R₂₈ each represent the same as defined in the formula (B1-1);

at least one combination of adjacent two or more of R₂₁₁ to R₂₁₈ are mutually bonded to form a substituted or unsubstituted monocyclic ring, mutually bonded to form a substituted or unsubstituted fused ring, or not mutually bonded;

R₂₁₁ to R₂₁₈ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and

R₉₀₁, R₉₀₂, R₉₀₃, R₉₀₄, R₉₀₅, R₉₀₆, R₉₀₇, R₈₀₁ and R₈₀₂ each represent the same as defined in the formula (B1).

It is preferable that R₂₁₁ to R₂₁₈ not being the bonding position to L_{A} do not form the substituted or unsubstituted monocyclic ring and do not form the substituted or unsubstituted fused ring.

In the organic EL device according to the exemplary embodiment, it is also preferable that R₂₁ or R₂₈ is the bonding position * to L_{A}.

In the organic EL device according to the exemplary embodiment, it is also preferable that R₂₂ or R₂₇ is the bonding position * to L_{A}.

In the organic EL device according to the exemplary embodiment, it is also preferable that R₂₃ or R₂₆ is the bonding position * to L_{A}.

In the organic EL device according to the exemplary embodiment, it is also preferable that R₂₄ or R₂₅ is the bonding position * to L_{A}.

In the organic EL device according to the exemplary embodiment, for instance, when R₂₅ is the bonding position * to L_{A}, the compound represented by the formula (B1) is represented by a formula (B1-3) below.

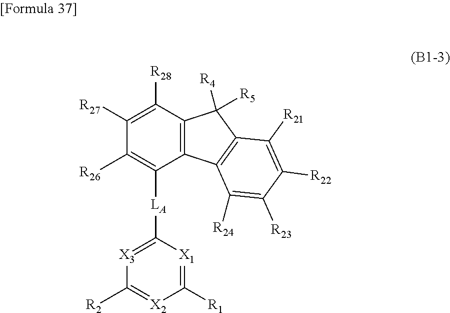

In the formula (B1-3):

X₁ to X₃, R₁ to R₃ and L_{A} each represent the same as defined in the formula (1); and

R₄, R₅, R₂₁ to R₂₄ and R₂₈ to R₂₈ each represent the same as defined in the formula (B1).

In the organic EL device according to the exemplary embodiment, it is preferable that A is a substituted or unsubstituted fused aryl group having 13 to 30 ring carbon atoms, or a substituted or unsubstituted fused heterocyclic group having 14 to 30 ring atoms.

In the organic EL device according to the exemplary embodiment, it is preferable that A is a substituted or unsubstituted fused aryl group having 13 to 20 ring carbon atoms, or a substituted or unsubstituted fused heterocyclic group having 14 to 20 ring atoms.

In the organic EL device according to the exemplary embodiment, it is preferable that A is a substituted or unsubstituted fused heterocyclic group having 14 to 20 ring atoms.

In the organic EL device according to the exemplary embodiment, it is also preferable that A is a fused heterocyclic group including two or more heteroatoms as ring atoms. Examples of the heteroatom include a nitrogen atom, oxygen atom, sulfur atom, silicon atom, phosphorus atom, and boron atom.

In the organic EL device according to the exemplary embodiment, the compound represented by the formula (1) is also preferably a compound represented by a formula (C1) below.

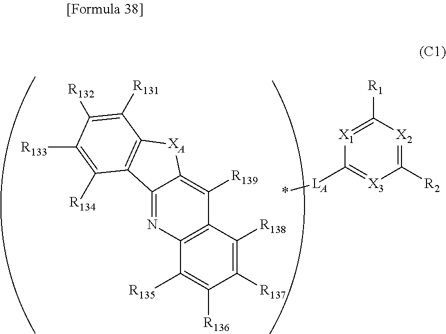

In the formula (C1):

X_{A} is an oxygen atom or a sulfur atom;

X₁ to X₃, R₁ to R₃ and L_{A} each represent the same as defined in the formula (1);

one of R₁₃1 to R₁₃₉ is a bonding position * to L_{A};

at least one combination of adjacent two or more of R₁₃₁ to R₁₃₉ not being the bonding position to L_{A} are mutually bonded to form a substituted or unsubstituted monocyclic ring, mutually bonded to form a substituted or unsubstituted fused ring, or not mutually bonded;

R₁₃₁ to R₁₃₉ not being the bonding position to L_{A}, not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and

R₉₀₁, R₉₀₂, R₉₀₃, R₉₀₄, R₉₀₅, R₉₀₆, R₉₀₇, R₈₀₁ and R₈₀₂ each represent the same as defined in the formula (A1).

In the organic EL device according to the exemplary embodiment, it is also preferable that one of X₁, X₂ and X₃ is a nitrogen atom.

In the organic EL device according to the exemplary embodiment, it is also preferable that X₂ is a nitrogen atom, X₁ and X₃ are each CR₃, R₃ represents the same as defined in the formula (1), and two R₃ are mutually the same or different.

In the organic EL device according to the exemplary embodiment, the compound represented by the formula (1) is also preferably a compound represented by a formula (1-N1) or (1-N11) below.

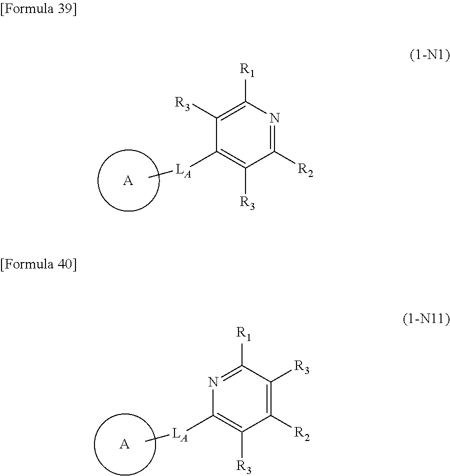

In the formulae (1-N1) and (1-N11): R₁, R₂, R₃, L_{A} and A each represent the same as defined in the formula (1).

In the organic EL device according to the exemplary embodiment, the compound represented by the formula (100) is also preferably a compound represented by a formula (1-N12) below.

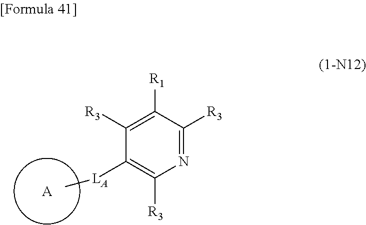

In the formula (1-N12): R₁, R₃, L_{A} and A each represent the same as defined in the formula (100).

In the organic EL device according to the exemplary embodiment, the compound represented by the formula (100) is also preferably a compound represented by a formula (A1-N12) below.

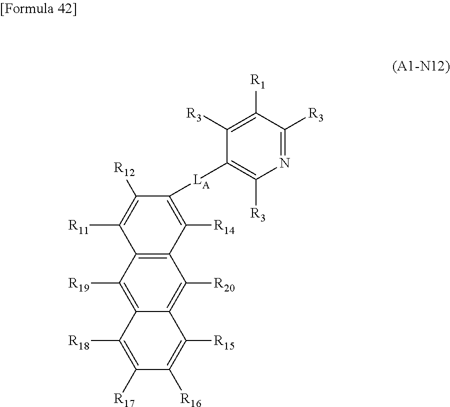

In the formula (A1-N12):

R₁, R₃ and L_{A} each represent the same as defined in the formula (100); and

R₁₁, R₁₂ and R₁₄ to R₂₀ each independently represent the same as defined in the formula (A1-1).

In the organic EL device according to the exemplary embodiment, the compound represented by the formula (1) is also preferably a compound represented by a formula (1-N2) or (1-N21) below.

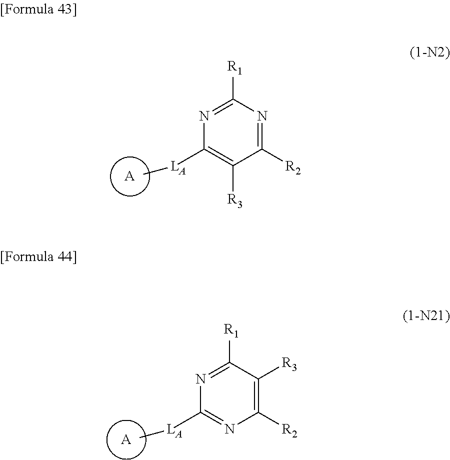

In the formulae (1-N2) and (1-N21): R₁, R₂, R₃, L_{A} and A each represent the same as defined in the formula (1).

In the organic EL device according to the exemplary embodiment, it is also preferable that X₁, X₂ and X₃ are each a nitrogen atom.

In the organic EL device according to the exemplary embodiment, the compound represented by the formula (1) is also preferably a compound represented by a formula (1-N3) below.

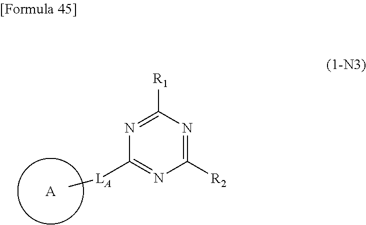

In the formula (1-N3): R₁, R₂, L_{A} and A each represent the same as defined in the formula (1).

In the organic EL device according to the exemplary embodiment, the compound represented by the formula (1) is also preferably a compound represented by a formula (A1-N3) below.

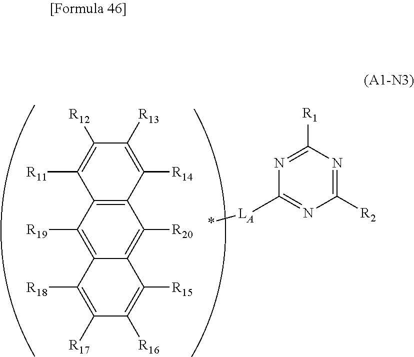

In the formula (A1-N3): R₁, R₂, L_{A}, R₁₁ to R₂₀ and * each represent the same as defined in the formula (A1).

In the organic EL device according to the exemplary embodiment, the compound represented by the formula (1) is also preferably a compound represented by a formula (A1-N31) or (A1-N32) below.

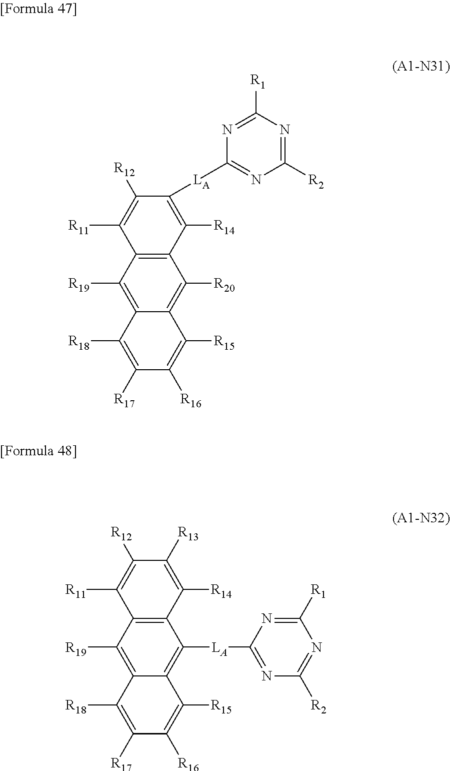

In the formulae (A1-N31) and (A1-N32):

R₁, R₂ and L_{A} each represent the same as defined in the formula (1); and

R₁₁ to R₂₀ each independently represent the same as defined in the formula (A1).

In the organic EL device according to the exemplary embodiment, the compound represented by the formula (1) is also preferably a compound represented by a formula (B1-N3) below.

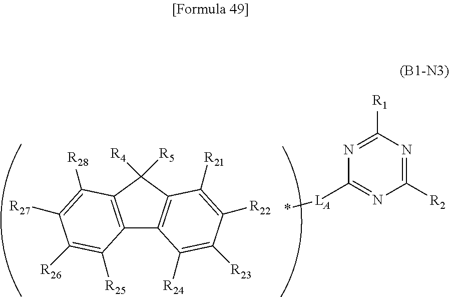

In the formula (B1-N3):

R₁, R₂ and L_{A} each represent the same as defined in the formula (1); and

R₄, R₅ and R₂₁ to R₂₈ each independently represent the same as defined in the formula (B1).

In the organic EL device according to the exemplary embodiment, it is preferable that R₁ and R₂ are each independently a substituted or unsubstituted aryl group having 6 to 50 ring carbon atoms.

In the organic EL device according to the exemplary embodiment, it is preferable that R₁ and R₂ are each independently a substituted or unsubstituted aryl group having 6 to 30 ring carbon atoms.

In the organic EL device according to the exemplary embodiment, it is also preferable that R₁ and R₂ are each independently a substituted or unsubstituted aryl group having 6 to 30 ring carbon atoms, and X₁, X₂ and X₃ are each a nitrogen atom.

In the organic EL device according to the exemplary embodiment, it is preferable that R₁₉ and R₂₀ are each independently a substituted or unsubstituted aryl group having 6 to 50 ring carbon atoms, or a substituted or unsubstituted heterocyclic group having 5 to 50 ring atoms, and

R₁ and R₂ are each independently a substituted or unsubstituted aryl group having 6 to 50 ring carbon atoms.

In the organic EL device according to the exemplary embodiment, it is preferable that R₁ and R₂ are each independently a substituted or unsubstituted aryl group having 6 to 18 ring carbon atoms.

In the organic EL device according to the exemplary embodiment, when X₁ is CR₃ and a combination of R₁ and R₃ are mutually bonded to form a substituted or unsubstituted monocyclic ring or mutually bonded to form a substituted or unsubstituted fused ring, the compound represented by the formula (1) is represented by a formula (1-P1) below.

In the organic EL device according to the exemplary embodiment, when X₂ is CR₃ and a combination of R₁ and R₃ are mutually bonded to form a substituted or unsubstituted monocyclic ring or mutually bonded to form a substituted or unsubstituted fused ring, the compound represented by the formula (1) is represented by a formula (1-P2) below.

In the organic EL device according to the exemplary embodiment, when X₂ is CR₃ and a combination of R₂ and R₃ are mutually bonded to form a substituted or unsubstituted monocyclic ring or mutually bonded to form a substituted or unsubstituted fused ring, the compound represented by the formula (1) is represented by a formula (1-P3) below.

In the organic EL device according to the exemplary embodiment, when X₃ is CR₃ and a combination of R₂ and R₃ are mutually bonded to form a substituted or unsubstituted monocyclic ring or mutually bonded to form a substituted or unsubstituted fused ring, the compound represented by the formula (1) is represented by a formula (1-P4) below.

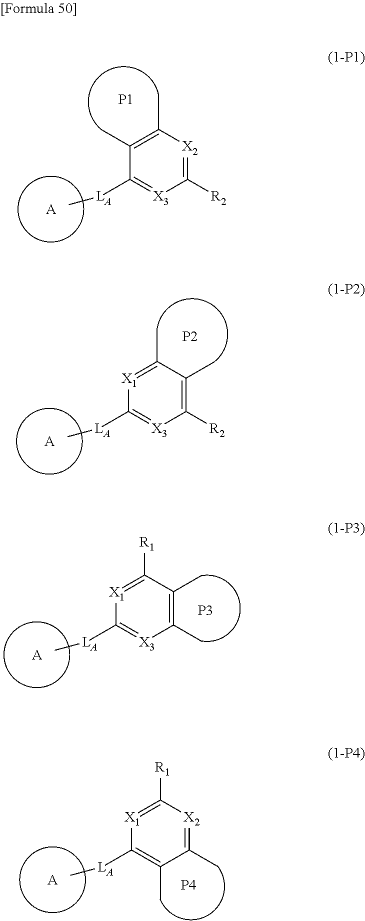

In the formulae (1-P1) to (1-P4):

a ring P1, a ring P2, a ring P3 and a ring P4 are each independently a substituted or unsubstituted monocyclic ring or a substituted or unsubstituted fused ring; and

X₁, X₂, X₃, R₁, R₂, R₃, L_{A} and A each represent the same as defined in the formula (1).

It is preferable that the ring P1, the ring P2, the ring P3 and the ring P4 are each independently a substituted or unsubstituted aromatic hydrocarbon ring or a substituted or unsubstituted aromatic heterocycle.

In the organic EL device according to the exemplary embodiment, the compound represented by the formula (1) is also preferably a compound represented by a formula (1-P11), (1-P21), (1-P31) or (1-P41) below.

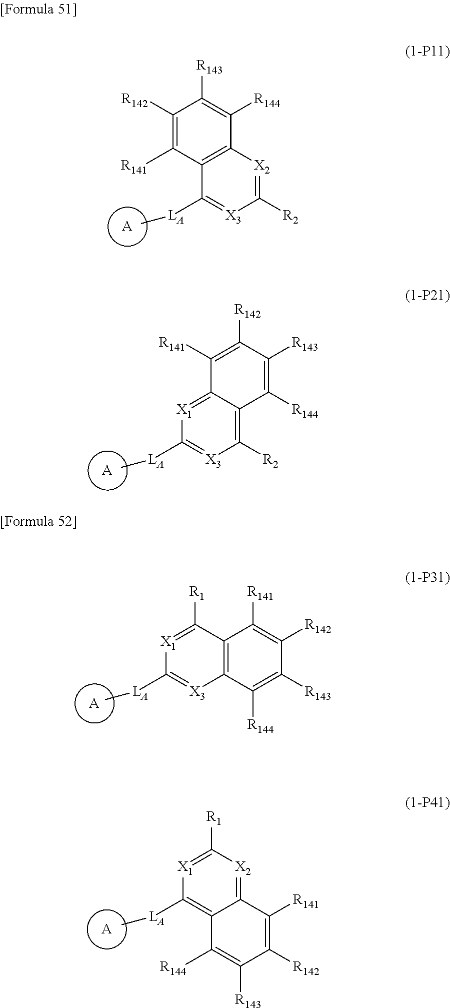

In the formulae (1-P11), (1-P21), (1-P31) and (1-P41):

R₁₄₁ to R₁₄₄ are each independently a hydrogen atom, a substituted or unsubstituted aryl group having 6 to 30 ring carbon atoms, or a substituted or unsubstituted heterocyclic group having 5 to 30 ring atoms; and

X₁, X₂, X₃, R₁, R₂, R₃, L_{A} and A each represent the same as defined in the formula (1).

In the organic EL device according to the exemplary embodiment, it is also preferable that L_{A} is a single bond.

In the organic EL device according to the exemplary embodiment, when L_{A} is a single bond, the compound represented by the formula (1) is represented by a formula (1-L1) below.

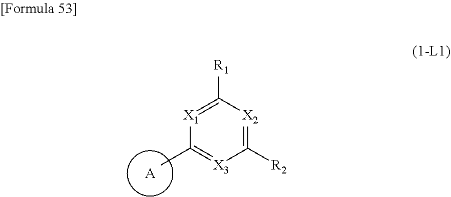

In the formula (1-L1): X₁, X₂, X₃, R₁, R₂, R₃ and A each represent the same as defined in the formula (1).

In the organic EL device according to the exemplary embodiment, L_{A} is also preferably a divalent group represented by a formula (L1-1), (L1-2) or (L1-3) below.

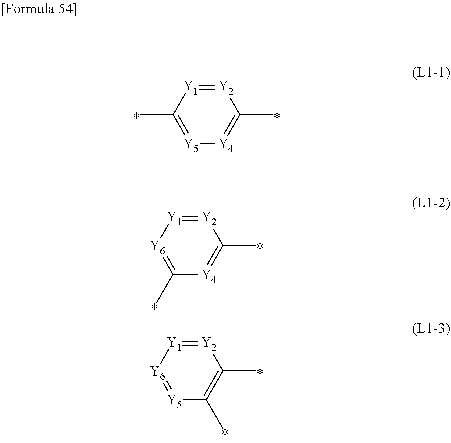

In the formulae (L1-1), (L1-2) and (L1-3):

Y₁ to Y₆ are each independently a nitrogen atom or CR₆;

R₆ is a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;

when a plurality of Re are present, the plurality of Re are mutually the same or different;

* represents a bonding position;

R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

when a plurality of R₉₀₁ are present, the plurality of R₉₀₁ are mutually the same or different;

when a plurality of R₉₀₂ are present, the plurality of R₉₀₂ are mutually the same or different;

when a plurality of R₉₀₃ are present, the plurality of R₉₀₃ are mutually the same or different;

when a plurality of R₉₀₄ are present, the plurality of R₉₀₄ are mutually the same or different;

when a plurality of R₉₀₅ are present, the plurality of R₉₀₅ are mutually the same or different;

when a plurality of R₉₀₆ are present, the plurality of R₉₀₆ are mutually the same or different;

when a plurality of R₉₀₇ are present, the plurality of R₉₀₇ are mutually the same or different;

when a plurality of R₈₀₁ are present, the plurality of R₈₀₁ are mutually the same or different; and

when a plurality of R₈₀₂ are present, the plurality of R₈₀₂ are mutually the same or different.

In the organic EL device according to the exemplary embodiment, the compound represented by the formula (1) is also preferably a compound represented by a formula (1-L2), (1-L3) or (1-L4) below.

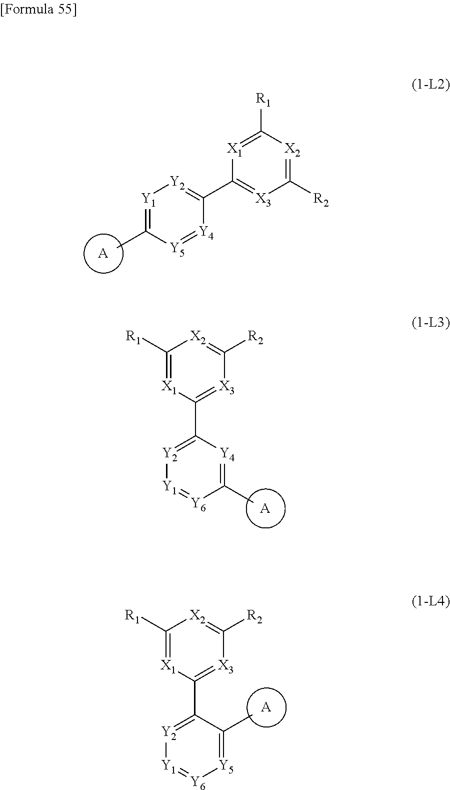

In the formulae (1-L2), (1-L3) and (1-L4): X₁, X₂, X₃, R₁, R₂, R₃ and A each represent the same as defined in the formula (1); and Y₁ to Y₆ represent the same as defined in the formulae (L1-1), (L1-2) and (L1-3).

In the organic EL device according to the exemplary embodiment, L_{A} is also preferably a divalent group represented by the formula (L1-1) or (L1-2).

In the organic EL device according to the exemplary embodiment, when L_{A} in the formula (A1-2) is a divalent group represented by the formula (L1-1), the compound represented by the formula (1) is represented by a formula (A1-L1) below.

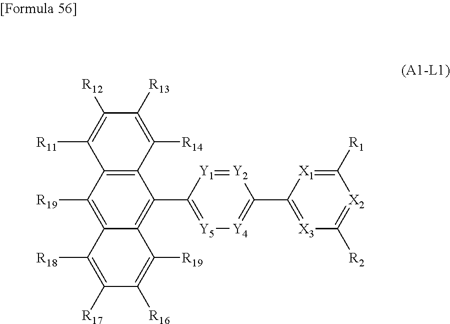

In the formula (A1-L1):

X₁ to X₃ and R₁ to R₃ each represent the same as defined in the formula (1);

R₁₁ to R₁₉ each independently represent the same as defined in the formula (A1-2); and

Y₁, Y₂, Y₄ and Y₅ each independently represent the same as defined in the formula (L1-1).

In the organic EL device according to the exemplary embodiment, when L_{A} in the formula (A1-1) is a divalent group represented by the formula (L1-2), the compound represented by the formula (1) is represented by a formula (A1-L2) below.

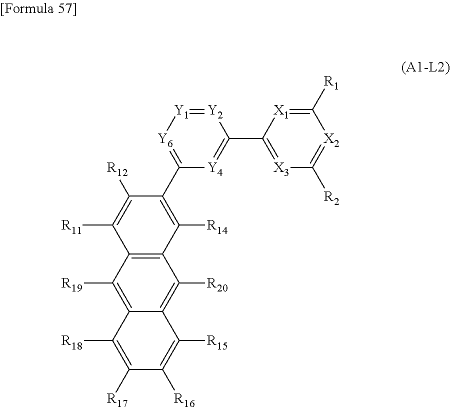

In the formula (A1-L2):

X₁ to X₃ and R₁ to R₃ each represent the same as defined in the formula (1);

R₁₁, R₁₂ and R₁₄ to R₂₀ each independently represent the same as defined in the formula (A1-1); and

Y₁, Y₂, Y₄ and Y₆ each independently represent the same as defined in the formula (L1-2).

In the organic EL device according to the exemplary embodiment, it is preferable that Y₁ to Y₆ not being the bonding position are each CR₆ and R₆ is a hydrogen atom.

In the organic EL device according to the exemplary embodiment, L_{A} is also preferably a divalent group represented by a formula (L1-1H), (L1-2H) or (L1-3H) below.

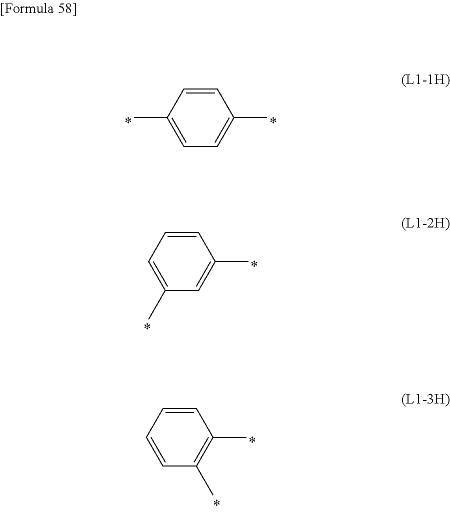

In the organic EL device according to the exemplary embodiment, L_{A} is also preferably a divalent group represented by the formula (L1-1H) or (L1-2H).

In the organic EL device according to the exemplary embodiment, it is preferable that all groups described as “substituted or unsubstituted” groups in the compound represented by the formula (100) are “unsubstituted” groups.

In the organic EL device according to the exemplary embodiment, it is preferable that all groups described as “substituted or unsubstituted” groups in the compound represented by the formula (1) are “unsubstituted” groups.

The content of the compound represented by the formula (100) in the first layer is preferably 90 mass % or more, more preferably 95 mass % or more, further preferably 99 mass % or more. Although it is not excluded that the first layer contains a material(s) other than the compound represented by the formula (100), the first layer contains no metal doping material.

It is also preferable that the first layer consists essentially of a compound represented by the formula (100). The “essentially” means that a minute amount of impurities or the like derived from a material for forming the first layer are unavoidably mixed.

It is also preferable that the first layer consists of a compound represented by the formula (100).

The content of the compound represented by the formula (1) in the first layer is preferably 90 mass % or more, more preferably 95 mass % or more, further preferably 99 mass % or more. Although it is not excluded that the first layer contains a material(s) other than the compound represented by the formula (1), the first layer contains no metal doping material.

It is also preferable that the first layer consists essentially of a compound represented by the formula (1). The “essentially” means that a minute amount of impurities or the like derived from a material for forming the first layer are unavoidably mixed.

It is also preferable that the first layer consists of a compound represented by the formula (1).

### Manufacturing Method of Compound Represented by Formula (100) and Compound Represented by Formula (1)

The compound represented by the formula (100) and the compound represented by the formula (1) can be manufactured by a known method. The compound represented by the formula (100) and the compound represented by the formula (1) can also be manufactured based on a known method through a known alternative reaction using a known material(s) tailored for the target compound.

### Specific Examples of Compound Represented by Formula (100) and Compound Represented by Formula (1)

Specific examples of the compound represented by the formula (100) and the compound represented by the formula (1) include the following compounds. However, the invention is not limited to these specific examples.

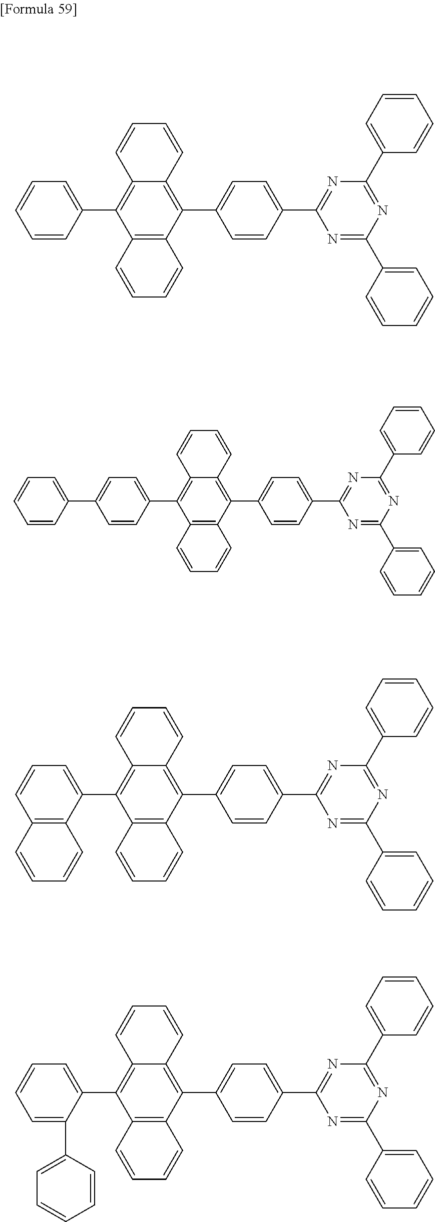 [SMILES: c1ccc(-c2nc(-c3ccccc3)nc(-c3ccc(-c4c5ccccc5c(-c5ccccc5)c5ccccc45)cc3)n2)cc1 ; c1ccc(-c2ccc(-c3c4ccccc4c(-c4ccc(-c5nc(-c6ccccc6)nc(-c6ccccc6)n5)cc4)c4ccccc34)cc2)cc1 ; c1ccc(-c2nc(-c3ccccc3)nc(-c3ccc(-c4c5ccccc5c(-c5cccc6ccccc56)c5ccccc45)cc3)n2)cc1 ; c1ccc(-c2nc(-c3ccccc3)nc(-c3ccc(-c4c5ccccc5c(-c5ccccc5-c5ccccc5)c5ccccc45)cc3)n2)cc1]

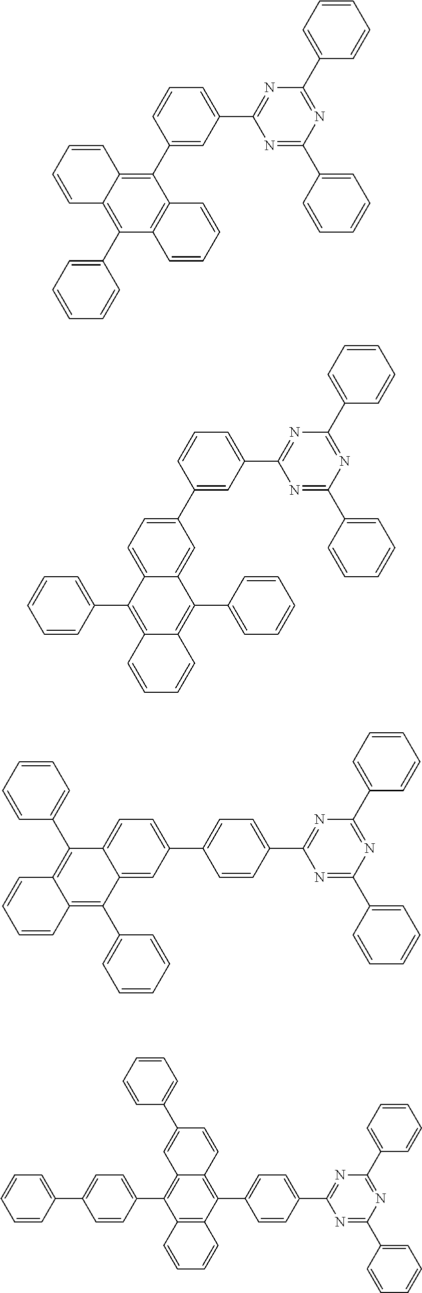 [SMILES: c1ccc(-c2nc(-c3ccccc3)nc(-c3cccc(-c4c5ccccc5c(-c5ccccc5)c5ccccc45)c3)n2)cc1 ; c1ccc(-c2nc(-c3ccccc3)nc(-c3cccc(-c4ccc5c(-c6ccccc6)c6ccccc6c(-c6ccccc6)c5c4)c3)n2)cc1 ; c1ccc(-c2nc(-c3ccccc3)nc(-c3ccc(-c4ccc5c(-c6ccccc6)c6ccccc6c(-c6ccccc6)c5c4)cc3)n2)cc1 ; c1ccc(-c2ccc(-c3c4ccccc4c(-c4ccc(-c5nc(-c6ccccc6)nc(-c6ccccc6)n5)cc4)c4ccc(-c5ccccc5)cc34)cc2)cc1]

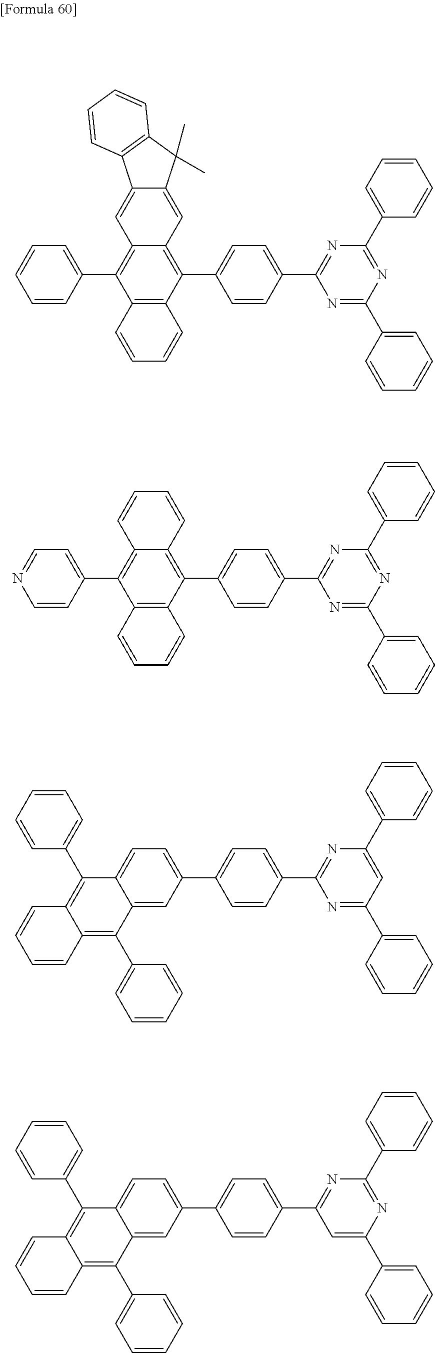 [SMILES: CC1(C)c2ccccc2-c2cc3c(-c4ccccc4)c4ccccc4c(-c4ccc(-c5nc(-c6ccccc6)nc(-c6ccccc6)n5)cc4)c3cc21 ; c1ccc(-c2nc(-c3ccccc3)nc(-c3ccc(-c4c5ccccc5c(-c5ccncc5)c5ccccc45)cc3)n2)cc1 ; c1ccc(-c2cc(-c3ccccc3)nc(-c3ccc(-c4ccc5c(-c6ccccc6)c6ccccc6c(-c6ccccc6)c5c4)cc3)n2)cc1 ; c1ccc(-c2cc(-c3ccc(-c4ccc5c(-c6ccccc6)c6ccccc6c(-c6ccccc6)c5c4)cc3)nc(-c3ccccc3)n2)cc1]

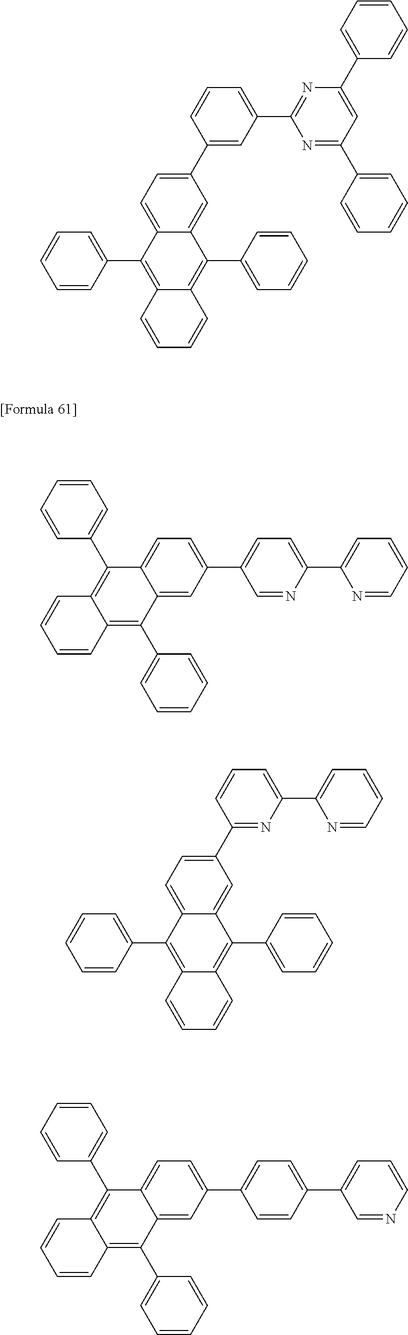 [SMILES: c1ccc(-c2cc(-c3ccccc3)nc(-c3cccc(-c4ccc5c(-c6ccccc6)c6ccccc6c(-c6ccccc6)c5c4)c3)n2)cc1 ; c1ccc(-c2c3ccccc3c(-c3ccccc3)c3cc(-c4ccc(-c5ccccn5)nc4)ccc23)cc1 ; c1ccc(-c2c3ccccc3c(-c3ccccc3)c3cc(-c4cccc(-c5ccccn5)n4)ccc23)cc1 ; c1ccc(-c2c3ccccc3c(-c3ccccc3)c3cc(-c4ccc(-c5cccnc5)cc4)ccc23)cc1]

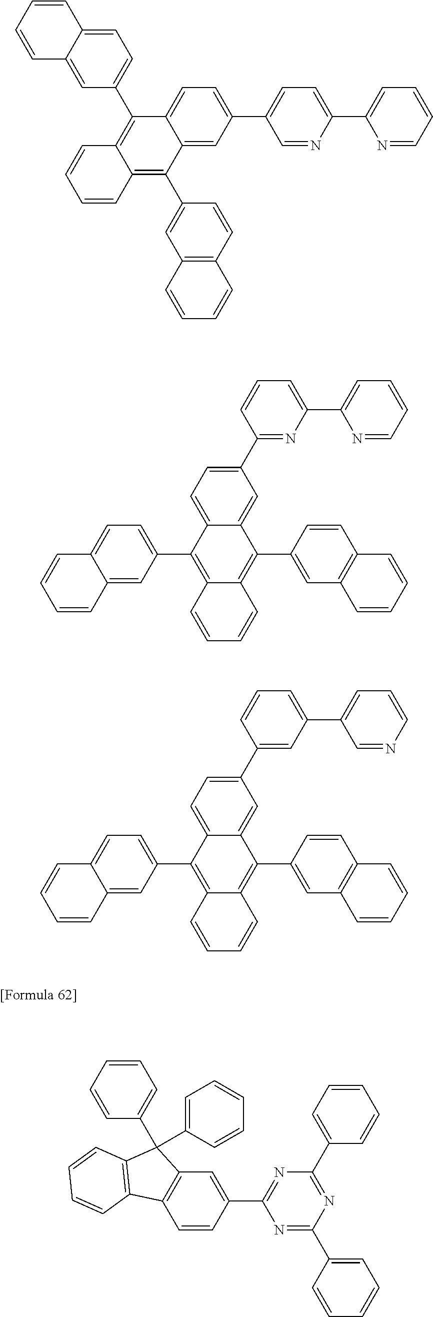 [SMILES: c1ccc(-c2ccc(-c3ccc4c(-c5ccc6ccccc6c5)c5ccccc5c(-c5ccc6ccccc6c5)c4c3)cn2)nc1 ; c1ccc(-c2cccc(-c3ccc4c(-c5ccc6ccccc6c5)c5ccccc5c(-c5ccc6ccccc6c5)c4c3)n2)nc1 ; c1cncc(-c2cccc(-c3ccc4c(-c5ccc6ccccc6c5)c5ccccc5c(-c5ccc6ccccc6c5)c4c3)c2)c1 ; c1ccc(-c2nc(-c3ccccc3)nc(-c3ccc4c(c3)C(c3ccccc3)(c3ccccc3)c3ccccc3-4)n2)cc1]

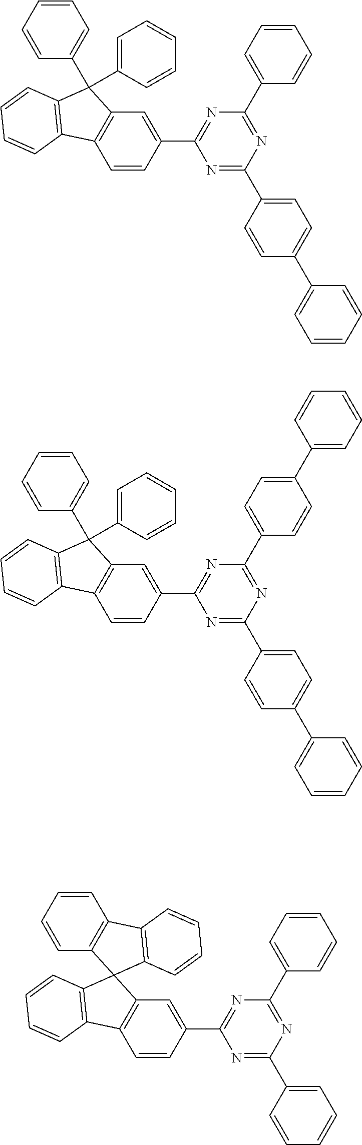 [SMILES: c1ccc(-c2ccc(-c3nc(-c4ccccc4)nc(-c4ccc5c(c4)C(c4ccccc4)(c4ccccc4)c4ccccc4-5)n3)cc2)cc1 ; c1ccc(-c2ccc(-c3nc(-c4ccc(-c5ccccc5)cc4)nc(-c4ccc5c(c4)C(c4ccccc4)(c4ccccc4)c4ccccc4-5)n3)cc2)cc1 ; c1ccc(-c2nc(-c3ccccc3)nc(-c3ccc4c(c3)C3(c5ccccc5-c5ccccc53)c3ccccc3-4)n2)cc1]

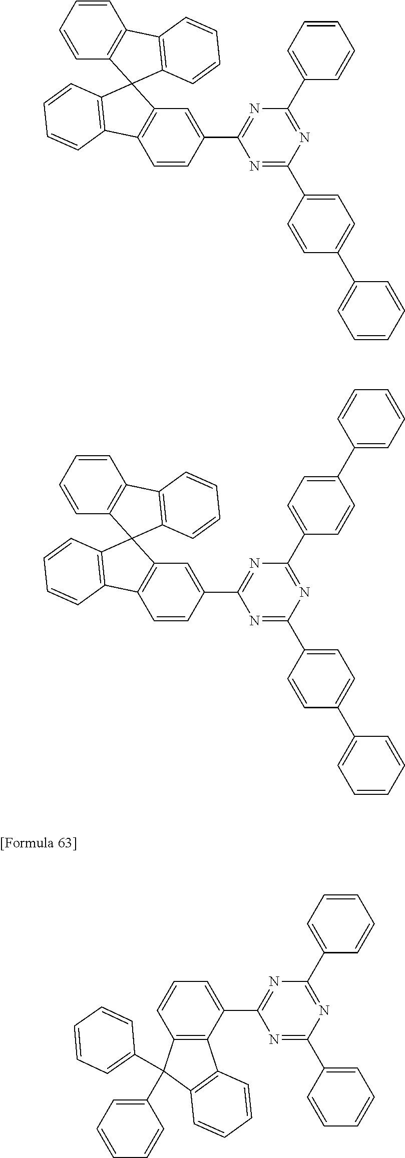 [SMILES: c1ccc(-c2ccc(-c3nc(-c4ccccc4)nc(-c4ccc5c(c4)C4(c6ccccc6-c6ccccc64)c4ccccc4-5)n3)cc2)cc1 ; c1ccc(-c2ccc(-c3nc(-c4ccc(-c5ccccc5)cc4)nc(-c4ccc5c(c4)C4(c6ccccc6-c6ccccc64)c4ccccc4-5)n3)cc2)cc1 ; c1ccc(-c2nc(-c3ccccc3)nc(-c3cccc4c3-c3ccccc3C4(c3ccccc3)c3ccccc3)n2)cc1]

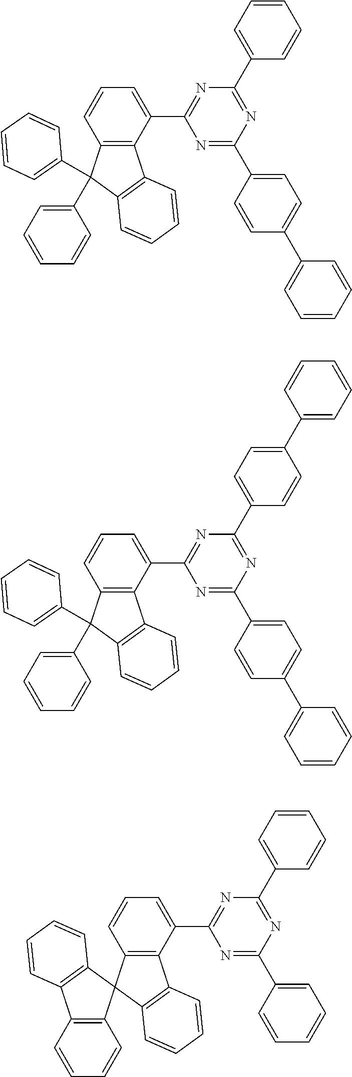

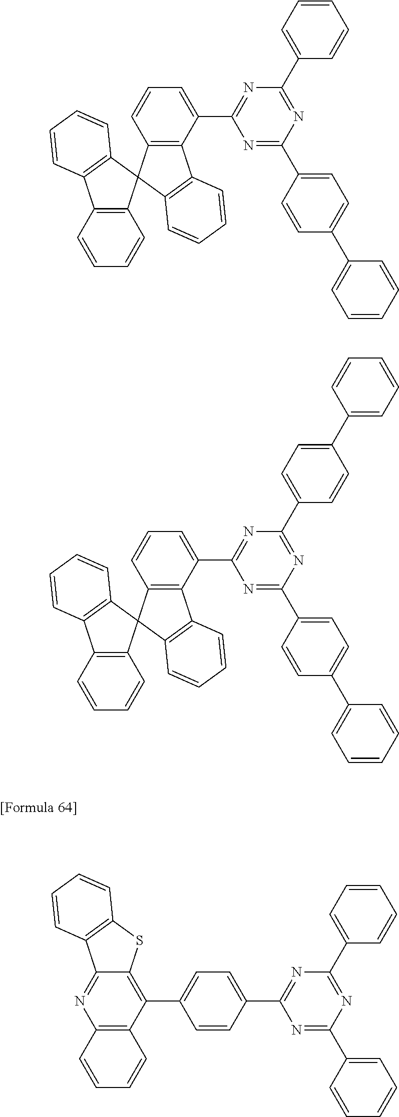

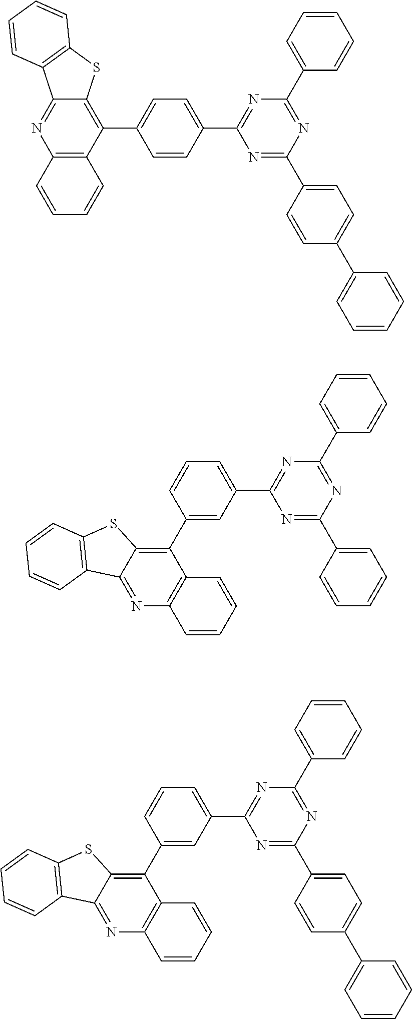 [SMILES: c1ccc(-c2ccc(-c3nc(-c4ccccc4)nc(-c4ccc(-c5c6ccccc6nc6c5sc5ccccc56)cc4)n3)cc2)cc1 ; c1ccc(-c2nc(-c3ccccc3)nc(-c3cccc(-c4c5ccccc5nc5c4sc4ccccc45)c3)n2)cc1 ; c1ccc(-c2ccc(-c3nc(-c4ccccc4)nc(-c4cccc(-c5c6ccccc6nc6c5sc5ccccc56)c4)n3)cc2)cc1]

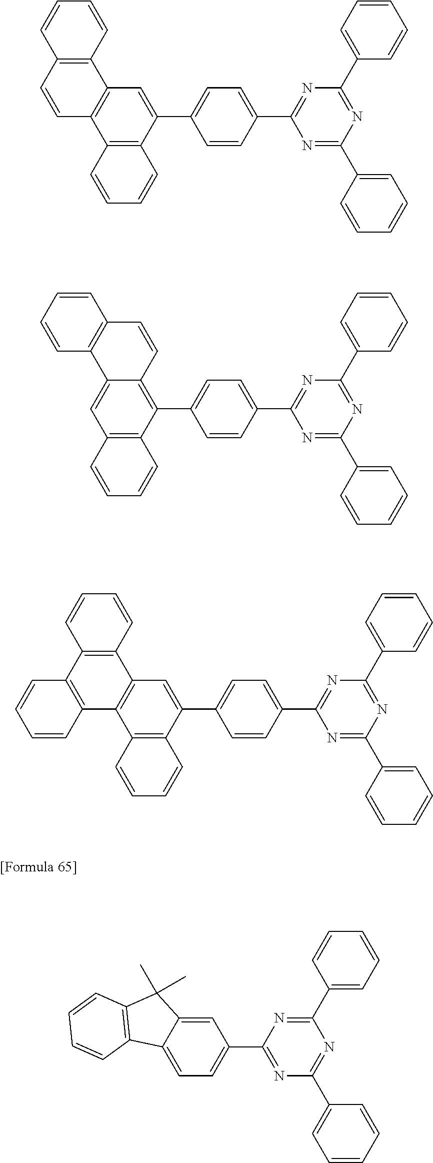 [SMILES: c1ccc(-c2nc(-c3ccccc3)nc(-c3ccc(-c4cc5c6ccccc6ccc5c5ccccc45)cc3)n2)cc1 ; c1ccc(-c2nc(-c3ccccc3)nc(-c3ccc(-c4c5ccccc5cc5c4ccc4ccccc45)cc3)n2)cc1 ; c1ccc(-c2nc(-c3ccccc3)nc(-c3ccc(-c4cc5c6ccccc6c6ccccc6c5c5ccccc45)cc3)n2)cc1 ; CC1(C)c2ccccc2-c2ccc(-c3nc(-c4ccccc4)nc(-c4ccccc4)n3)cc21]

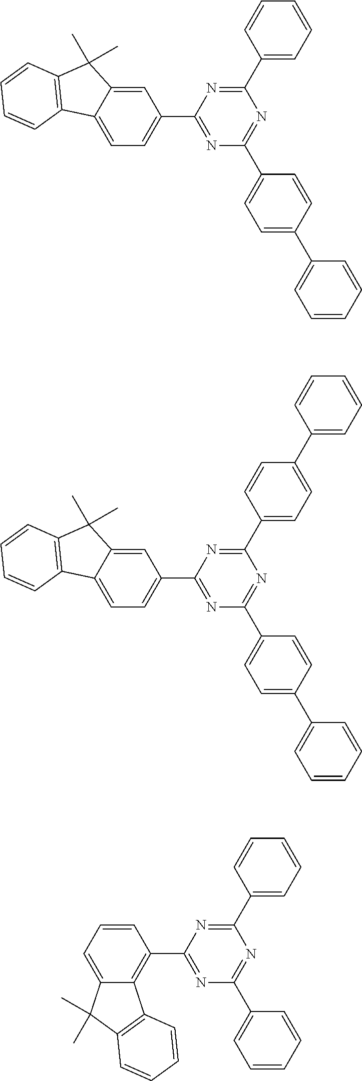 [SMILES: CC1(C)c2ccccc2-c2ccc(-c3nc(-c4ccccc4)nc(-c4ccc(-c5ccccc5)cc4)n3)cc21 ; CC1(C)c2ccccc2-c2ccc(-c3nc(-c4ccc(-c5ccccc5)cc4)nc(-c4ccc(-c5ccccc5)cc4)n3)cc21 ; CC1(C)c2ccccc2-c2c(-c3nc(-c4ccccc4)nc(-c4ccccc4)n3)cccc21]

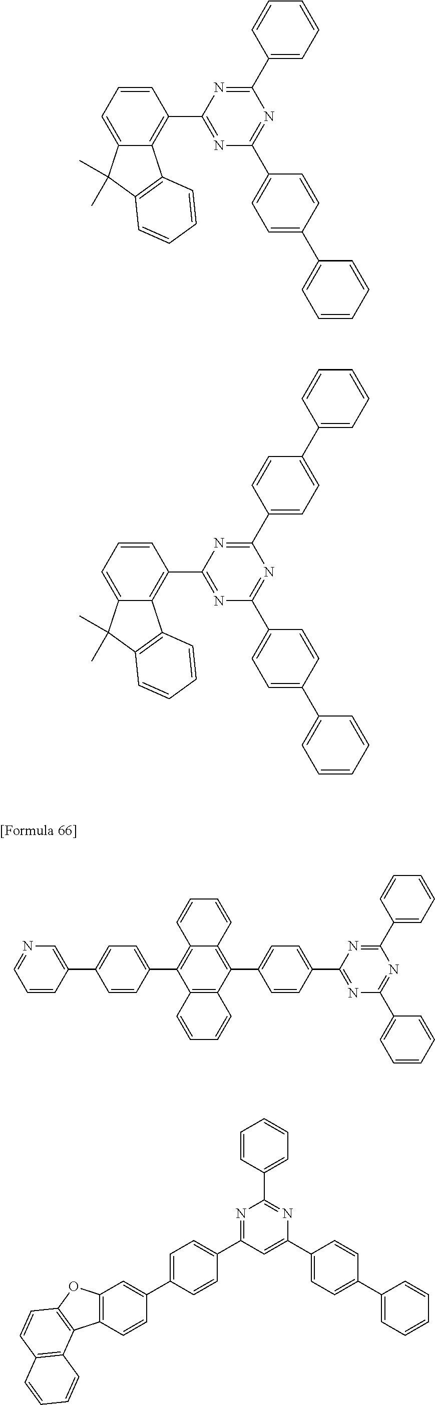 [SMILES: CC1(C)c2ccccc2-c2c(-c3nc(-c4ccccc4)nc(-c4ccc(-c5ccccc5)cc4)n3)cccc21 ; CC1(C)c2ccccc2-c2c(-c3nc(-c4ccc(-c5ccccc5)cc4)nc(-c4ccc(-c5ccccc5)cc4)n3)cccc21 ; c1ccc(-c2nc(-c3ccccc3)nc(-c3ccc(-c4c5ccccc5c(-c5ccc(-c6cccnc6)cc5)c5ccccc45)cc3)n2)cc1 ; c1ccc(-c2ccc(-c3cc(-c4ccc(-c5ccc6c(c5)oc5ccc7ccccc7c56)cc4)nc(-c4ccccc4)n3)cc2)cc1]

### Resonator Structure

It is preferable that the organic EL device according to the exemplary embodiment has a resonator structure whose order of interference is first order between the light reflection layer and the semitransmissive electrode as the cathode.

For instance, when the organic EL device 1 has a resonator structure whose order of interference is first order, the organic EL device 1 specifically has a resonator structure whose order of interference is first order between the light reflection layer 31 and the semitransmissive electrode 4. A distance D₃ between the light reflection layer 31 and the semitransmissive electrode 4 in the organic EL device 1 corresponds to a sum of a thickness of the hole transporting zone 6, a thickness of the emitting layer 5 and a thickness of the electron transporting zone 7. It is preferable that the organic EL device 1A, the organic EL device 1B and the organic EL device 1C also have a resonator structure whose order of interference is first order in the same manner as the organic EL device 1.

The resonator structure of the organic EL device will be described below.

The organic EL device has a resonator structure in which emitted light is made to resonate between the light reflection layer 31 and the semitransmissive electrode 4 to be extracted, whereby color purity of the extracted light can be improved and intensity of the extracted light near a central wavelength of resonance can be improved.

In a resonator structure in which a reflective end surface of the light reflection layer 31 close to the emitting layer 5 is defined as a first end P1, a reflective end surface of the semitransmissive electrode 4 close to the emitting layer 5 is defined as a second end P2, the organic layer (the hole transporting zone 6, the emitting layer 5 and the electron transporting zone 7) is defined as a resonance portion, and light generated in the emitting layer 5 is made to resonate and extracted from the second end P2, an optical distance L between the first end P1 and the second end P2 of the resonator is set so as to satisfy a numerical formula (OP1) below. It is preferable that the optical distance L is actually selected so as to be a positive minimum value that satisfies the numerical formula (OP1).

$$\begin{matrix}[ Numerical Formula 1 ] & \\ \frac{2 L}{λ} + \frac{Φ}{2 π} = m & ( OP1 )\end{matrix}$$

Symbols in the numerical formula (OP1) are described as follows.

L represents the optical distance between the first end P1 and the second end P2.

Φ represents a sum (Φ=Φ1+Φ2) of phase shift Φ1 of reflected light generated on the first end P1 and phase shift 02 of reflected light generated on the second end P2. A unit of the phase shift is rad.

λ represents a peak wavelength of a spectrum of light to be extracted from the second end P2.

m represents an integer to make L positive. m corresponds to the order of interference. When m is 1, the organic EL device has a resonator structure whose order of interference is first order.

In the numerical formula (OP1), L and Δ only need to be represented by a common unit. The unit of L and Δ is, for instance, nm.

The optical distance L is a total sum (=n₁d₁+n₂d₂+ . . . ) of optical film thicknesses (=refractive index (n)×film thickness (d)) of the organic layer between the light reflection layer 31 and the semitransmissive electrode 4. It should be noted that, when the light actually reflects on the light reflection layer 31 and the semitransmissive electrode 4, the sum Φ of the phase shifts changes depending on a combination of electrode materials and organic materials of which reflective interfaces are formed.

In the organic EL device according to the exemplary embodiment, it is preferable that an optical distance L₁ between the maximum emission position of the emitting layer 5 and the first end P1 and an optical distance L₂ between the maximum emission position and the second end P2 are adjusted to respectively satisfy a numerical formula (OP2) and a numerical formula (OP3) below. Herein, the maximum emission position refers to a position where the luminous intensity is the largest in an emitting region. For instance, when the emitting layer 5 emits light on both the interface close to the light reflection layer 31 and the interface close to the semitransmissive electrode 4, the maximum emission position is defined by the interface having the larger luminous intensity between the interfaces.

$$\begin{matrix}[ Numerical Formula 2 ] & \\ \begin{matrix}{L}_{1} = {tL}_{1} + {a}_{1} \\ \frac{2 {tL}_{1}}{λ} = - \frac{{Φ}_{1}}{2 π} + {m}_{1}\end{matrix} \} & ( OP2 )\end{matrix}$$

Symbols in the numerical formula (OP2) are described as follows.

tL₁ represents an optical theoretical distance between the first end P1 and the maximum emission position.

a₁ represents a correction amount based on an emission distribution in the emitting layer 5.

λ represents a peak wavelength of a spectrum of light to be extracted.

Φ₁ represents a phase shift of reflected light generated on the first end P1. A unit of Φ₁ is rad.

m₁ is 0 or an integer. In the organic EL device according to the exemplary embodiment, m₁ is preferably 0. A position of the optical distance L₁ when m₁ is 0 corresponds to a “zero-order interference position” viewed from the light reflection layer 31.

$$\begin{matrix}[ Numerical Formula 3 ] & \\ \begin{matrix}{L}_{2} = {tL}_{2} + {a}_{2} \\ \frac{2 {tL}_{2}}{λ} = - \frac{{Φ}_{2}}{2 π} + {m}_{2}\end{matrix} \} & ( OP3 )\end{matrix}$$

Symbols in the numerical formula (OP3) are described as follows.

tL₂ represents an optical theoretical distance between the second end P2 and the maximum emission position.

a₂ represents a correction amount based on the emission distribution in the emitting layer 5.

λ represents a peak wavelength of a spectrum of light to be extracted.

Φ₂ represents a phase shift of reflected light generated on the second end P2. A unit of Φ₂ is rad.

m₂ is 0 or an integer. m₂ is preferably 1.

It is more preferable that m₁ is 0 and m₂ is 1. A position of the optical distance L₂ when m₂ is 1 corresponds to a “first-order interference position” viewed from the semitransmissive electrode 4.

The numerical formula (OP2) represents conditions for realizing a relationship in which, when a light toward the light reflection layer 31, which is among the lights generated in the emitting layer 5, is reflected on the first end P1 to return, a phase of the return light and a phase at the time of emission become the same and therefore the return light and a light toward the semitransmissive electrode 4, which is among the lights generated in the emitting layer 5, enhance each other.

The numerical formula (OP3) represents conditions for realizing a relationship in which, when a light toward the semitransmissive electrode 4, which is among the lights generated in the emitting layer 5, is reflected on the second end P2 to return, a phase of the return light and a phase at the time of emission become the same and therefore the return light and a light toward the light reflection layer 31, which is among the lights generated in the emitting layer 5, enhance each other.

The organic EL device according to the exemplary embodiment can be designed so that m₁ in the numerical formula (OP2) and m₂ in the numerical formula (OP3) satisfy m₂>m₁ by forming the electron transporting zone 7 to have a larger film thickness than the hole transporting zone 6. By designing so that m₂>m₁ is satisfied, the organic EL device according to the exemplary embodiment has an improved viewing angle.

The optical theoretical distance tL₁ in the numerical formula (OP2) and the optical theoretical distance tL₂ in the numerical formula (OP3) are theoretical values showing that, when the emission region is considered not to spread, an amount of phase change on the first end P1 or the second end P2 is offset by an amount of phase change due to light progress, so that the phase of the return light and the phase at the time of emission become the same. However, since the emission region usually spreads, the correction amounts a₁ and a₂ based on the emission distribution are added to the numerical formula (OP2) and numerical formula (OP3), respectively.

Although the correction amounts a₁ and a₂ differ depending the emission distribution, when the maximum emission position is on the interface of the emitting layer 5 close to the semitransmissive electrode 4 and the emission distribution spreads from the maximum emission position toward the light reflection layer 31, or when the maximum emission position is on the interface of the emitting layer 5 close to the light reflection layer 31 and the emission distribution spreads from the maximum emission position toward the semitransmissive electrode 4, the correction amounts a₁ and a₂ can be calculated by a numerical formula (OP4) below.

$$\begin{matrix}[ Numerical Formula 4 ] & \\ \begin{matrix}{a}_{1} = b ( {log}_{e} ( s ) ) \\ {a}_{2} = - {a}_{1}\end{matrix} \} & ( OP4 )\end{matrix}$$

Symbols in the numerical formula (OP4) are described as follows.

b is a value within a range of 2n≤b≤6n when the emission distribution in the emitting layer 5 spreads from the maximum emission position toward the light reflection layer 31. b is a value within a range of −6n≤b≤−2n when the emission distribution in the emitting layer 5 spreads from the maximum emission position toward the semitransmissive electrode 4.

s represents a physical property value (1/e decay distance) relating to the emission distribution in the emitting layer 5.

n is an average refractive index between the first end P1 and the second end P2 at the peak wavelength λ of the spectrum of the light to be extracted.

The above description is of the resonator structure of the organic EL device.

### Measurement Method of Film Thickness of Layer or Zone

A thickness (film thickness) of each of the layers or the zones included in the organic EL device can be measured as follows.

A central portion of an organic EL device having a layer or a zone (i.e., measurement target) is cut in a perpendicular direction to a plane where the layer or the zone of the measurement target is formed (i.e., a thickness direction of an organic layer). The cut surface of the central portion is observed with a transmission electron microscope (TEM) to measure a film thickness of the layer or the zone.

For instance, a film thickness of an emitting layer of the organic EL device is measured. In this case, a central portion of the organic EL device having the layer to be measured is cut in a perpendicular direction to a plane where the emitting layer is formed (i.e., a thickness direction of the emitting layer). The cut surface of the central portion is observed with a transmission electron microscope (TEM) to determine the film thickness. For instance, the central portion of the organic EL device is represented by CL in FIGS. 1 to 4.

It should be noted that the central portion of the organic EL device means a central portion of a shape of the organic EL device projected through the semitransmissive electrode. When the projected shape is, for instance, rectangular, the central portion of the organic EL device means an intersection of the diagonal lines of the rectangle.

Herein, when a target zone or layer includes a plurality of layers, a thickness means a sum of thicknesses of the plurality of layers.

### Light Reflection Layer

The light reflection layer 31 is in direct contact with the transparent electrode 32.

Reflectance on the interface of the light reflection layer 31 with the transparent electrode 32 is preferably 50% or more, more preferably 80% or more.

The light reflection layer 31 is preferably a metal layer. Metals forming the metal layer are not particularly limited. Examples of the metals include metal selected from the group consisting of gold (Au), platinum (Pt), nickel (Ni), tungsten (W), chrome (Cr), molybdenum (Mo), iron (Fe), cobalt (Co), copper (Cu), palladium (Pd), titanium (Ti), and silver (Ag), and alloys containing a plurality of types of metals selected from the group consisting of these metals. The light reflection layer 31 is exemplified by an APC layer. APC refers to an alloy of silver (Ag), palladium (Pd), and copper (Cu). A material usable for the light reflection layer 31 is not limited to the above materials.

### Transparent Electrode

The transparent electrode 32 is interposed between the light reflection layer 31 and the hole transporting zone 6.

The transparent electrode 32 is in direct contact with the light reflection layer 31. The transparent electrode 32 is preferably in direct contact with the hole transporting zone 6.

The transparent electrode 32 is preferably a transparent conductive film. Examples of the transparent conductive film as the transparent electrode 32 include an Indium Tin Oxide (ITO) film and an indium zinc oxide film. A compound usable for the transparent electrode is not limited to the above compounds.

Transmittance of the transparent electrode 32 is preferably 50% or more, more preferably 80% or more. The transmittance of the transparent electrode 32 is preferably 100% or less. From the viewpoint of suppressing decay due to multiple reflections, an extinction coefficient of the transparent electrode 32 is preferably 0.05 or less, more preferably 0.01 or less.

The film thickness of the transparent electrode 32 is preferably 15 nm or less.

The film thickness of the transparent electrode 32 is preferably 5 nm or more.

The film thickness of the transparent electrode 32 can be measured by the above-described “Measurement Method of Film Thickness of Layer or Zone.” Since the film thickness of the transparent electrode 32 is 15 nm or less, the film thickness of the hole transporting zone 6 can be increased while a sum of the film thickness of the hole transporting zone 6 and the film thickness of the transparent electrode 32 is kept less than 40 nm. The film thickness of the transparent electrode 32 being 5 nm or more enables stable hole injection.

### Hole Transporting Zone

The hole transporting zone 6 is at least interposed between the transparent electrode 32 and the emitting layer 5.

The film thickness of the hole transporting zone 6 is preferably 10 nm or more and less than 25 nm, more preferably in a range from 10 nm to 20 nm.

The film thickness of the hole transporting zone 6 can be measured by the above-described “Measurement Method of Film Thickness of Layer or Zone.”

The sum of the film thickness of the transparent electrode 32 and the film thickness of the hole transporting zone 6 in the organic EL device according to the exemplary embodiment is preferably less than 40 nm.

When the sum of the film thickness of the transparent electrode 32 and the film thickness of the hole transporting zone 6 in the organic EL device according to the exemplary embodiment is less than 40 nm, the viewing angle can be improved.

The sum of the film thickness of the transparent electrode 32 and the film thickness of the hole transporting zone 6 in the organic EL device according to the exemplary embodiment is preferably 15 nm or more.

The hole transporting zone means a region where holes are transferred. Hole mobility μ^{H} in the hole transporting zone is preferably 10⁻⁶ [cm²/(V·s)] or more. The hole mobility μ^{H} [cm²/(V·s)] can be measured according to impedance spectroscopy disclosed in JP 2014-110348 A.

The hole transporting zone 6 also preferably consists of a single layer.

The hole transporting zone 6 also preferably includes a plurality of layers.

Examples of the layer forming the hole transporting zone 6 includes a hole injecting layer, a hole transporting layer, and an electron blocking layer.

In the organic EL devices 1, 1A, 1B, 1C shown in FIGS. 1 to 4 as examples of the organic EL device according to the exemplary embodiment, the hole transporting zone 6 includes the hole injecting layer 61 and the hole transporting layer 62.

### Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the highly hole-injectable substance further include: an aromatic amine compound, which is a low-molecule organic compound, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,3-f:20,30-h]quinoxaline-2,3,6,7,10,11-hexacarbonitrile (HAT-CN).

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) are also usable.

A compound usable for the hole injecting layer is not limited to the above compounds.

### Hole Transporting Layer

The hole transporting layer is a layer containing a highly hole-transporting substance. An aromatic amine compound, carbazole derivative, anthracene derivative and the like are usable for the hole transporting layer. Specific examples of a material for the hole transporting layer include an aromatic amine compound such as 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)trphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(V-s) or more.

For the hole transporting layer, a carbazole derivative such as CBP, 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used. A high polymer compound such as poly(N-vinylcarbazole) (abbreviation: PVK) and poly(4-vinyltiphenylamine) (abbreviation: PVTPA) is also usable.

A compound usable for the hole transporting layer is not limited to the above compounds.

However, in addition to the above substances, any substance exhibiting a higher hole transportability than an electron transportability may be used for the hole transporting layer. A layer including the highly hole-transporting substance may be provided in the form of a single layer or a laminate of two or more layers.

### Emitting Layer

### Guest Material of Emitting Layer

The emitting layer, which is a layer containing a highly luminescent substance, can contain various materials. For instance, a fluorescent compound that emits fluorescence and a phosphorescent compound that emits phosphorescence are usable as the highly luminescent substance. The fluorescent compound is a compound emittable from a singlet state. The phosphorescent compound is a compound emittable from a triplet state. The guest material is occasionally referred to as a dopant material, emitter or luminescent material.

Examples of a blue fluorescent material usable for the emitting layer include a pyrene derivative, styrylamine derivative, chrysene derivative, fluoranthene derivative, fluorene derivative, diamine derivative, and triarylamine derivative. Specific examples of the blue fluorescent material include N,N′-bis[4-(9H-carbazole-9-yl)phenyl]-N,N′-diphenylstilbene-4,4′-diamine (abbreviation: YGA2S), 4-(9H-carbazole-9-yl)-4′-(10-phenyl-9-anthryl)triphenylamine (abbreviation: YGAPA), and 4-(10-phenyl-9-anthryl)-4′-(9-phenyl-9H-carbazole-3-yl)triphenylamine (abbreviation: PCBAPA).

Examples of a green fluorescent material usable for the emitting layer include an aromatic amine derivative. Specific examples of the green fluorescent material include N-(9,10-diphenyl-2-anthryl)-N,9-diphenyl-9H-carbazole-3-amine (abbreviation: 2PCAPA), N-[9,10-bis(1,1′-biphenyl-2-yl)-2-anthryl]-N,9-diphenyl-9H-carbazole-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylene diamine (abbreviation: 2DPABPhA), N-[9,10-bis(1,1′-biphenyl-2-yl)]-N-[4-(9H-carbazole-9-yl)phenyl]-N-phenylanthracene-2-amine (abbreviation: 2YGABPhA), and N,N,9-triphenylanthracene-9-amine (abbreviation: DPhAPhA).

Examples of a red fluorescent material usable for the emitting layer include a tetracene derivative and a diamine derivative. Specific examples of the red fluorescent material include N,N,N′,N′-tetrakis(4-methylphenyl)tetracene-5,11-diamine (abbreviation: p-mPhTD), and 7,14-diphenyl-N,N,N′,N′-tetrakis(4-methylphenyl)acenaphtho[1,2-a]fluoranthene-3,10-diamine (abbreviation: p-mPhAFD).

Examples of a blue phosphorescent material usable for the emitting layer include metal complexes such as an iridium complex, osmium complex and platinum complex. Specific examples of the blue phosphorescent material include bis[2-(4′,6′-difluorophenyl)pyridinato-N,C2′]iidium(III)tetrakis(1-pyrazolyl)borate (abbreviation: FIr6), bis[2-(4′,6′-difluorophenyl)pyridinato-N,C2′]iridium(III)picolinate (abbreviation: Flrpic), bis[2-(3′,5′bistrifluoromethylphenyl)pyrdinato-N,C2′]iridium(III)picolinate (abbreviation: Ir(CF₃ppy)₂(pic)), and bis[2-(4′,6′-difluorophenyl)pyridinato-N,C2′]iridium(III)acetylacetonato (abbreviation: Flracac).

Examples of a green phosphorescent material usable for the emitting layer include an iridium complex. Specific examples of the green phosphorescent material include tris(2-phenylpyridinato-N,C2′)iridium(III) (abbreviation: Ir(ppy)₃), bis(2-phenylpyridinato-N,C2′)iridium(III)acetylacetonato (abbreviation: Ir(ppy)₂(acac)), bis(1,2-diphenyl-1H-benzimidazolato)iridium(III)acetylacetonato (abbreviation: Ir(pbi)₂(acac)), and bis(benzo[h]quinolinato)iridium(III)acetylacetonato (abbreviation: Ir(bzq)₂(acac)).

Examples of a red phosphorescent material usable for the emitting layer include metal complexes such as an iridium complex, platinum complex, terbium complex, and europium complex. Specific examples of the red phosphorescent material include an organic metal complex such as bis[2-(2′-benzo[4,5-a]thienyl)pyridinato-N,C3′]iridium(III)acetylacetonato (abbreviation: Ir(btp)₂(acac)), bis(1-phenylisoquinolinato-N,C2′)iridium(III)acetylacetonato (abbreviation: Ir(piq)₂(acac)), (acetylacetonato)bis[2,3-bis(4-fluorophenyl)quinoxalinato]iridium(III) (abbreviation: Ir(Fdpq)₂(acac)), and 2,3,7,8,12,13,17,18-octaethyl-21H,23H-porphyrinplatinum(II) (abbreviation: PtOEP).

Moreover, since a rare-earth metal complex, examples of which include tris(acetylacetonato)(monophenanthroline)terbium(III) (abbreviation: Tb(acac)₃(Phen)), tris(1,3-diphenyl-1,3-propanedionato)(monophenanthroline)europium(III) (abbreviation: Eu(DBM)₃(Phen)), and tris[1-(2-thenoyl)-3,3,3-trifluoroacetonato](monophenanthroline)europium(III) (abbreviation: Eu(TTA)₃(Phen)), emits light from rare-earth metal ions (electron transition between different multiplicities), the rare-earth metal complex is usable as a phosphorescent compound.

### Host Material of Emitting Layer

The emitting layer may include the above-described highly luminescent substance (guest material) dispersed in another substance (host material). The substance for dispersing the highly luminescent substance may be various substances, preferably a substance having higher Lowest Unoccupied Molecular Orbital (LUMO level) and lower Highest Occupied Molecular Orbital (HOMO level) than the highly luminescent substance.

Herein, the “host material” refers to, for instance, a material that accounts for “50 mass % or more of the layer.” Moreover, for instance, the “host material” may accounts for 60 mass % or more of the layer, 70 mass % or more of the layer, 80 mass % or more of the layer, 90 mass % or more of the layer, or 95 mass % or more of the layer.

Examples of the substance (host material) for dispersing the highly luminescent substance include (1) a metal complex such as an aluminum complex, beryllium complex or zinc complex; (2) a heterocyclic compound such as an oxadiazole derivative, benzimidazole derivative or phenanthroline derivative; (3) a fused aromatic compound such as a carbazole derivative, anthracene derivative, phenanthrene derivative, pyrene derivative or chrysene derivative; and (4) an aromatic amine compound such as a triarylamine derivative or a fused polycyclic aromatic amine derivative.

As the metal complex, specifically, tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), bis[2-(2-benzothiazolyl)phenolato]zinc(II) (abbreviation: ZnBTZ), or the like is usable.

As the heterocyclic compound, specifically, 2-(4-biphenylyl)-5-(4-tert-butylphenyl)-1,3,4-oxadiazole (abbreviation: PBD), 1,3-bis[5-(p-tert-butylphenyl)-1,3,4-oxadiazole-2-yl]benzene (abbreviation: OXD-7), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), bathophenanthroline (abbreviation: BPhen), bathocuproine (abbreviation: BCP), or the like is usable.

As the fused aromatic compound, specifically, 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 3,3′,3″-(benzene-1,3,5-triyl)tripyrene (abbreviation: TPB3), 9,10-diphenylanthracene (abbreviation: DPAnth), 6,12-dimethoxy-5,11-diphenylchrysene, or the like is usable.

As the aromatic amine compound, specifically, N,N-diphenyl-9-[4-(10-phenyl-9-anthryl)phenyl]-9H-carbazole-3-amine (abbreviation: CzA1PA), 4-(10-phenyl-9-anthryl)triphenylamine (abbreviation: DPhPA), N,9-diphenyl-N-[4-(10-phenyl-9-anthryl)phenyl]-9H-carbazole-3-amine (abbreviation: PCAPA), N,9-diphenyl-N-{4-[4-(10-phenyl-9-anthryl)phenyl]phenyl}-9H-carbazole-3-amine (abbreviation: PCAPBA), N-(9,10-diphenyl-2-anthryl)-N,9-diphenyl-9H-carbazole-3-amine (abbreviation: 2PCAPA), NPB (or a-NPD), TPD, DFLDPBi, BSPB, or the like is usable.

Moreover, a plurality of types of the substance (host material) for dispersing the highly luminescent substance (guest material) can be used.

A compound usable for the emitting layer is not limited to the above compounds.

Herein, blue light emission refers to a light emission in which a main peak wavelength of emission spectrum is in a range from 430 nm to 500 nm.

A main peak wavelength of the blue fluorescent compound is preferably in a range from 430 nm to 500 nm, more preferably 430 nm or more and less than 500 nm.

Herein, green light emission refers to a light emission in which a main peak wavelength of emission spectrum is in a range from 500 nm to 560 nm.

A main peak wavelength of the green fluorescent compound is preferably in a range from 500 nm to 560 nm, more preferably in a range from 500 nm to 540 nm, further preferably in a range from 510 nm to 530 nm.

Herein, red light emission refers to a light emission in which a main peak wavelength of emission spectrum is in a range from 600 nm to 660 nm.

A main peak wavelength of the red fluorescent compound is preferably in a range from 600 nm to 660 nm, more preferably in a range from 600 nm to 640 nm, further preferably in a range from 600 nm to 630 nm.

Herein, the main peak wavelength means a peak wavelength of an emission spectrum exhibiting a maximum luminous intensity among emission spectra measured in a toluene solution in which a measurement target compound is dissolved at a concentration ranging from 10- mol/to 10⁻⁵ mol/l. A spectrophotofluorometer (F-7000 manufactured by Hitachi High-Tech Science Corporation) is used as a measurement device.

The emitting layer also preferably contains no phosphorescent material as a dopant material.

Further, the emitting layer also preferably contains neither a heavy metal complex nor a phosphorescent rare-earth metal complex. Examples of the heavy metal complex herein include iridium complex, osmium complex, and platinum complex.

Further, the emitting layer also preferably contains no metal complex.

### Electron Transporting Zone

The electron transporting zone 7 is at least interposed between the emitting layer 5 and the semitransmissive electrode 4.

The electron transporting zone 7 is in direct contact with the emitting layer 5 and also in direct contact with the semitransmissive electrode 4.

The film thickness of the electron transporting zone 7 is preferably 50 nm or more, more preferably 100 nm or more, further preferably 120 nm or more.

The film thickness of the electron transporting zone 7 is preferably 160 nm or less.

The film thickness of the electron transporting zone can be measured by the above-described “Measurement Method of Film Thickness of Layer or Zone.”

The electron transporting zone 7 means a region where electrons are transferred. Electron mobility μ^{E} in the electron transporting zone 7 is preferably 10⁻⁶ [cm²/(V·s)] or more. The electron mobility μ^{E} [cm²/(V·s)] can be measured according to impedance spectroscopy disclosed in JP 2014-110348 A.

The electron transporting zone 7 may be provided by a single layer or a plurality of layers. Specifically, the electron transporting zone 7 in the organic EL device according to the exemplary embodiment may be a zone including a single layer or a zone including a plurality of layers.

Examples of the layer forming the electron transporting zone 7 includes an electron injecting layer, an electron transporting layer, and a hole blocking layer.

In the organic EL device according to the exemplary embodiment, the first layer is also preferably an electron transporting layer.

In the organic EL device according to the exemplary embodiment, the first layer is also preferably a hole blocking layer.

In the organic EL device according to the exemplary embodiment, the second layer is also preferably an electron transporting layer.

In the organic EL device according to the exemplary embodiment, the second layer is also preferably a hole blocking layer.

In the organic EL device according to the exemplary embodiment, the third layer is also preferably an electron transporting layer.

In the organic EL device according to the exemplary embodiment, the third layer is also preferably an electron injecting layer.

The thickness of the second layer is preferably smaller than the thickness of the first layer.

The thickness of the second layer is preferably 3 nm or more, more preferably 4 nm or more, further preferably 5 nm or more.

The thickness of the second layer is preferably 20 nm or less, more preferably 15 nm or less, further preferably 10 nm or less.

A thickness of the third layer is preferably smaller than the thickness of the first layer.

The thickness of the third layer is preferably 3 nm or more, more preferably 4 nm or more, further preferably 5 nm or more.

The thickness of the third layer is preferably 20 nm or less, more preferably 15 nm or less, further preferably 10 nm or less.

To supplement electron injectability of the first layer, the third layer is preferably a layer that contains an organic compound having a group with high electron injectability. Examples of the group with high electron injectability include an azole group typified by benzimidazole and triazole, an azine group typified by pyridine and phenanthroline, a phosphine oxide group typified by diphenylphosphine oxide, and a cyano group.

Further, the third layer is also preferably an organic compound layer containing an alkali metal, an alkaline earth metal, a compound of an alkali metal or a compound of an alkaline earth metal, which are described below for the electron injecting layer.

Further, the third layer also preferably contains a compound having at least one group selected from the group consisting of an azole group, an azine group, a phosphine oxide group and a cyano group.

The compound having a benzazole group is represented by, for instance, a formula (70) below.

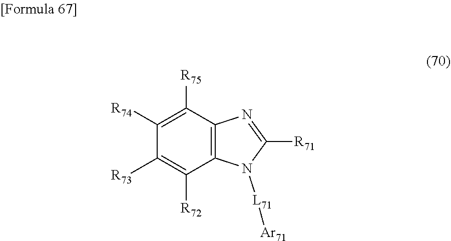

In the formula (70):

R₇₁ to R₇₅ are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms;

L₇₁ is a substituted or unsubstituted arylene group having 6 to 50 ring carbon atoms, or a substituted or unsubstituted divalent heterocyclic group having 5 to 50 ring atoms; and

Ar₇₁ is a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

### Specific Examples of Compound Represented by Formula (70)

Specific examples of the compound represented by the formula (70) include compounds shown below. However, the invention is not limited to these specific examples.

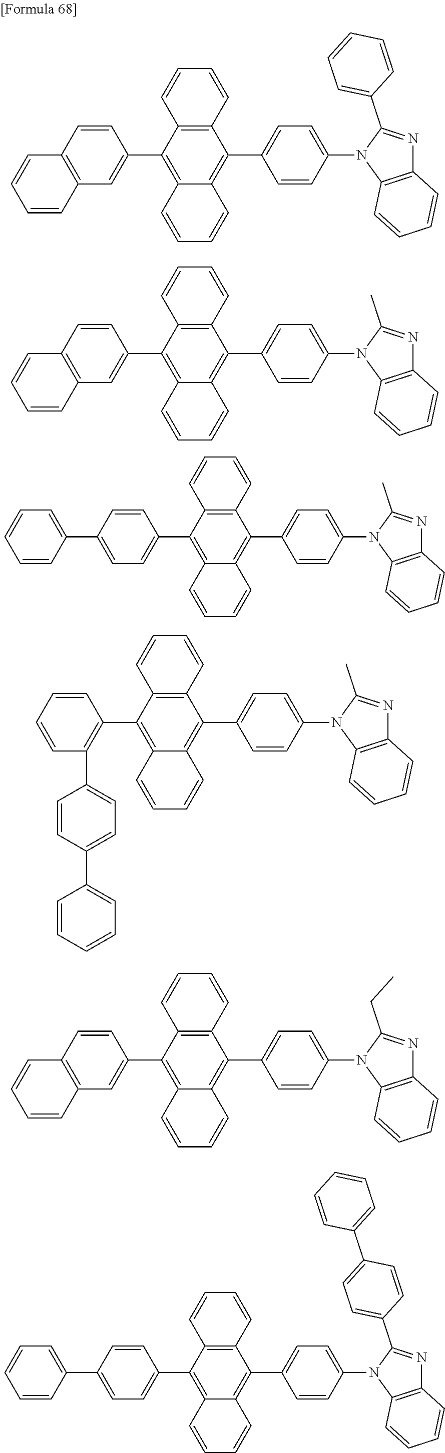

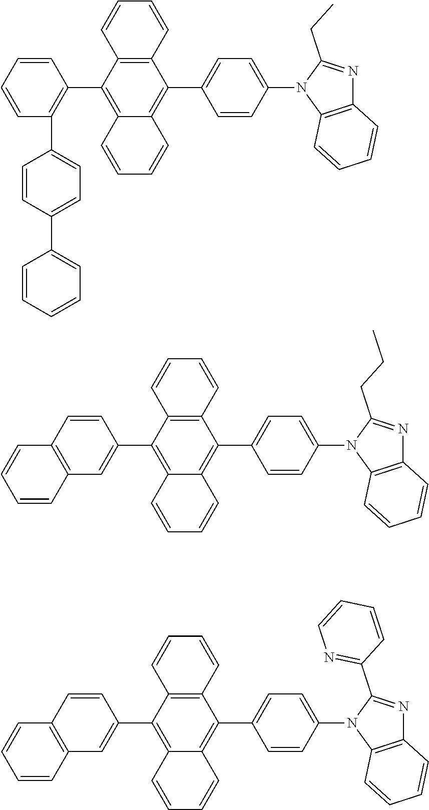 [SMILES: CCc1nc2ccccc2n1-c1ccc(-c2c3ccccc3c(-c3ccccc3-c3ccc(-c4ccccc4)cc3)c3ccccc23)cc1 ; CCCc1nc2ccccc2n1-c1ccc(-c2c3ccccc3c(-c3ccc4ccccc4c3)c3ccccc23)cc1 ; c1ccc(-c2nc3ccccc3n2-c2ccc(-c3c4ccccc4c(-c4ccc5ccccc5c4)c4ccccc34)cc2)nc1]

The electron transporting layer is a layer containing a highly electron-transporting substance. For the electron transporting layer, (1) a metal complex such as an aluminum complex, beryllium complex, and zinc complex, (2) a hetero aromatic compound such as imidazole derivative, benzimidazole derivative, azine derivative, carbazole derivative, and phenanthroline derivative, and (3) a high polymer compound are usable. Specifically, as a low-molecule organic compound, a metal complex such as Alq, tris(4-methyl-8-quinolinato)aluminum (abbreviation: Almq₃), bis(10-hydroxybenzo[h]quinolinato)beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In the organic EL device according to the exemplary embodiment, for instance, a benzimidazole compound is suitably usable for the third layer as the electron transporting layer. The above-described substances usable for the electron transporting layer mostly have an electron mobility of 10⁻⁶ cm²/(V·s) or more. It should be noted that any substance other than the above substance may be used for the electron transporting layer as long as the substance exhibits a higher electron transportability than the hole transportability.

Further, a high polymer compound is usable for the electron transporting layer. For instance, poly[(9,9-dihexylfluorene-2,7-diyl)-co-(pyridine-3,5-diyl)](abbreviation: PF-Py), poly[(9,9-dioctylfluorene-2,7-diyl)-co-(2,2′-bipyridine-6,6′-diyl)](abbreviation: PF-BPy) and the like are usable.

### Electron Injecting Layer

The electron injecting layer is a layer containing a highly electron-injectable substance. Examples of a material for the electron injecting layer include an alkali metal, an alkaline earth metal, a rare earth metal, a compound of an alkali metal, a compound of an alkaline earth metal and a compound of a rare earth metal, examples of which include lithium (Li), cesium (Cs), calcium (Ca), ytterbium (Yb), lithium fluoride (LiF), (8-quinolinolato)lithium (Liq), cesium fluoride (CsF), calcium fluoride (CaF₂), and lithium oxide (LiOx). Further, the electron injecting layer also preferably contains a substance exhibiting electron transportability and an alkali metal, an alkaline earth metal, a rare earth metal, a compound of an alkali metal, a compound of an alkaline earth metal, or a compound of a rare earth metal. As a combination of such a substance exhibiting electron transportability and metal or a metal compound, for instance, magnesium (Mg) added to Alq (Tris(8-hydroxyquinoline)aluminum) may be used. When the electron injecting layer contains a substance exhibiting electron transportability and metal or a metal compound, electrons are efficiently injected to the electron injecting layer from the cathode.

Alternatively, the electron injecting layer may be provided by a composite material in a form of a mixture of an organic compound and an electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable as the electron donor. Further, the organic compound such as tetrathiafulvalene (abbreviation: TTF) is also usable as the electron donor.

A compound usable for the electron transporting layer, the electron injecting layer and the emitting layer is not limited to the above compounds.

### Semitransmissive Electrode

The semitransmissive electrode 4 transmits light and also reflects light on an interface with the electron transporting zone 7. Transmittance of the semitransmissive electrode 4 is preferably 50% or more.

A film thickness of the semitransmissive electrode 4 is preferably in a range from 5 nm to 30 nm.

The semitransmissive electrode 4 is preferably formed of an elemental metal or an alloy of a metal material. In the case of a metal material having a large extinction coefficient, a transmitted light amount decreases due to light absorption when light is transmitted through the semitransmissive electrode 4. In order to efficiently extract light from the semitransmissive electrode 4, it is preferable to suppress light absorption. Thus, as the material of the semitransmissive electrode 4, it is preferable to select an elemental metal or an alloy of a metal material having a small real part refractive index. Examples of the metal material include silver, aluminum, magnesium, calcium, sodium and gold. A material usable for the semitransmissive electrode is not limited to the above materials.

In the organic EL device according to the exemplary embodiment, a reflective electrode is defined by at least the light reflection layer 31 and the transparent electrode 32. For instance, the organic EL device according to the exemplary embodiment is a so-called top emission type organic EL device. In the organic EL device according to the exemplary embodiment, the reflective electrode is provided on the substrate 2 and light is extracted from the opposite semitransmissive electrode 4 across the organic layer. In the organic EL device according to the exemplary embodiment, the reflective electrode is the anode and the semitransmissive electrode 4 is the cathode.

### Capping Layer

The organic EL device according to the exemplary embodiment may include a capping layer. The capping layer is preferably provided on an upper side of the semitransmissive electrode serving as the cathode. The capping layer is preferably in direct contact with the semitransmissive electrode. The organic EL devices 1, 1A, 1B, 1C shown in FIGS. 1 to 4 each include the capping layer 8.

When the organic EL device according to the exemplary embodiment is of a top emission type, the organic EL device preferably includes the capping layer.

Examples of the material of the capping layer include a polymer compound, metal oxide, metal fluoride, metal boride, silicon nitride and a silicon compound (e.g., silicon oxide).

Moreover, examples of the material of the capping layer include an aromatic amine derivative, anthracene derivative, pyrene derivative, fluorene derivative, and dibenzofuran derivative. A compound usable for the capping layer is not limited to the above compounds.

Further, the organic EL device according to the exemplary embodiment may include, as the capping layer, a laminate of a plurality of layers containing the material used for the capping layer.

### Substrate

The substrate 2 is a support supporting the organic EL device. Examples of a material of the substrate 2 include glass, quartz and plastic. A flexible substrate is also usable as the substrate 2. The flexible substrate is a bendable substrate. Examples of the flexible substrate include a plastic substrate made of polycarbonate, polyarylate, polyether sulfone, polypropylene, polyester, polyvinylfluoride or polyvinyl chloride. Moreover, an inorganic vapor deposition film is also usable as the substrate 2.

### Layer Thickness

In the organic EL device according to the exemplary embodiment, the film thickness of each layer forming the organic layer included between the reflective electrode as the anode and the semitransmissive electrode 4 is not particularly limited unless otherwise specified herein. In general, an excessively small film thickness of each layer forming the organic layer is likely to cause defects (e.g. pin holes) and an excessively large thickness thereof requires application of high voltage to deteriorate the efficiency. The film thickness of each layer forming the organic layer is typically preferably in a range from several nm to 1 μm.

### Layer Formation Method

The method of forming each layer of the organic EL device according to the exemplary embodiment is not limited except as specifically described above, but a known method such as a dry film-forming method or a wet film-forming method can be adopted. Examples of the dry film-forming include vacuum deposition, sputtering, plasma process, and ion plating. Examples of the wet film-forming include spin coating, dipping, flow coating and ink-jet.

According to the exemplary embodiment, an organic electroluminescence device drivable at a low voltage even if an electron transporting material in a thickened electron transporting zone is not doped with active metal can be provided. Reasons are described below.

Conventionally, by doping an electron transporting material with active metal in a thickened electron transporting zone, a drive voltage of an organic EL device has been decreased. Doping with the active metal has, for instance, the following disadvantages (i) to (iv): (i) Resistance of the electron transporting zone is decreased and consequently leaks are easily caused between pixels adjacent to each other; (ii) Light emission is deactivated by diffusion of the active metal; (iii) EL emission is made with an organic material colored by interaction between the metal and the organic material, or light radiated from the emitting layer is absorbed by such a colored organic material; and (iv) A lifetime is reduced due to excessive electrons supplied from the electron transporting zone to the emitting layer.

In particular, the organic EL device that uses a zero-order optical interference position (constructive interference position) from the anode is excellent in viewing angle and luminous efficiency, but needs to have a thickened electron transporting zone. With the thickened electron transporting zone, the organic EL device is liable to have a high drive voltage.

In the organic EL device according to the exemplary embodiment, the first layer in the electron transporting zone is thickened to have a film thickness of 50 nm or more and contains no metal doping material. However, the first layer contains a compound represented by the formula (100) or a compound represented by the formula (1). This allows the organic EL device according to the exemplary embodiment to be drivable at a low voltage. Further, the organic EL device according to the exemplary embodiment is drivable at a low voltage regardless of an emission color of the emitting layer. Moreover, according to an arrangement of the compound represented by the formula (100) or the compound represented by the formula (1), the electron transporting zone containing any of these compounds can supplement electron injection into the emitting layer from the cathode.

### Second Exemplary Embodiment

### Electronic Device

An electronic device according to a second exemplary embodiment is installed with any one of the organic EL devices according to the above exemplary embodiment. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light.

### Modification of Embodiment(s)

The scope of the invention is not limited by the above-described exemplary embodiments but includes any modification and improvement as long as such modification and improvement are compatible with the invention.

For instance, the emitting layer is not limited to a single layer, but may be provided by laminating a plurality of emitting layers. When the organic EL device includes the plurality of the emitting layers, the emitting layers may be each independently, for instance, a fluorescent emitting layer, or a phosphorescent emitting layer with use of emission caused by electron transfer from the triplet excited state directly to the ground state.

When the organic EL device includes a plurality of emitting layers, these emitting layers may be mutually adjacently provided, or may form a so-called tandem organic EL device, in which a plurality of emitting units are layered via an intermediate layer.

For instance, a blocking layer may be provided adjacent to at least one of a side of the emitting layer close to the anode or a side of the emitting layer close to the cathode. The blocking layer is preferably provided in contact with the emitting layer to block at least any of holes, electrons, excitons or combinations thereof.

For instance, when the blocking layer is provided in contact with the side of the emitting layer close to the cathode, the blocking layer permits transport of electrons and blocks holes from reaching a layer provided closer to the cathode (e.g., the electron transporting layer) beyond the blocking layer. When the organic EL device includes the electron transporting layer, the blocking layer is preferably interposed between the emitting layer and the electron transporting layer.

When the blocking layer is provided in contact with the side of the emitting layer close to the anode, the blocking layer permits transport of holes and blocks electrons from reaching a layer provided closer to the anode (e.g., the hole transporting layer) beyond the blocking layer. When the organic EL device includes the hole transporting layer, the blocking layer is preferably interposed between the emitting layer and the hole transporting layer.

Alternatively, the blocking layer may be provided adjacent to the emitting layer so that excitation energy does not leak out from the emitting layer toward neighboring layer(s). The blocking layer blocks excitons generated in the emitting layer from being transferred to a layer(s) (e.g., the electron transporting layer and the hole transporting layer) closer to the electrode(s) beyond the blocking layer.

The emitting layer is preferably bonded with the blocking layer.

Specific structure, shape and the like of the components in the invention may be designed in any manner as long as an object of the invention can be achieved.

### EXAMPLES

Example(s) of the invention will be described below. However, the invention is not limited to Example(s).

### Compounds

Compounds represented by the formula (100) or (1) and used for manufacturing organic EL devices in Examples and Comparatives are shown below.

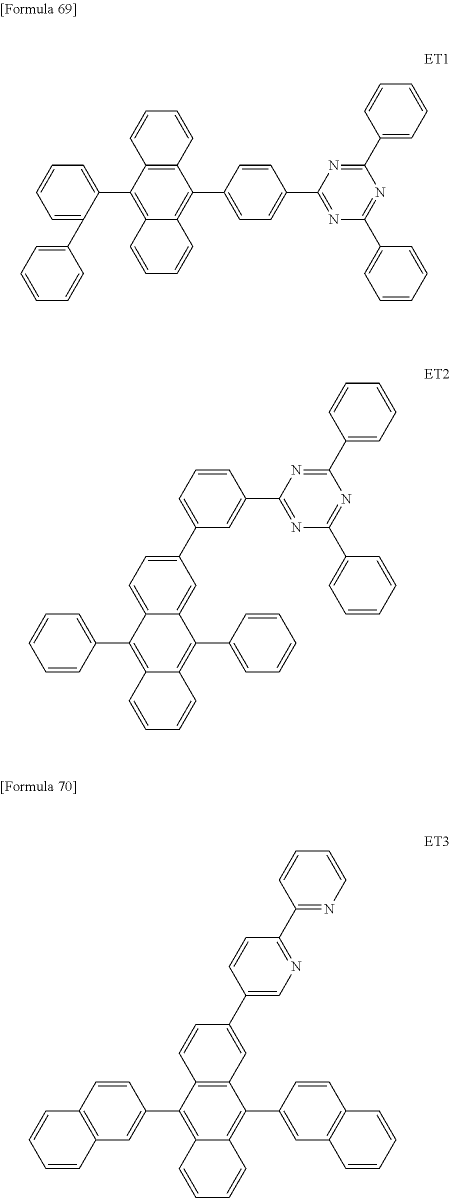 [SMILES: c1ccc(-c2nc(-c3ccccc3)nc(-c3ccc(-c4c5ccccc5c(-c5ccccc5-c5ccccc5)c5ccccc45)cc3)n2)cc1 ; c1ccc(-c2nc(-c3ccccc3)nc(-c3cccc(-c4ccc5c(-c6ccccc6)c6ccccc6c(-c6ccccc6)c5c4)c3)n2)cc1 ; c1ccc(-c2ccc(-c3ccc4c(-c5ccc6ccccc6c5)c5ccccc5c(-c5ccc6ccccc6c5)c4c3)cn2)nc1]

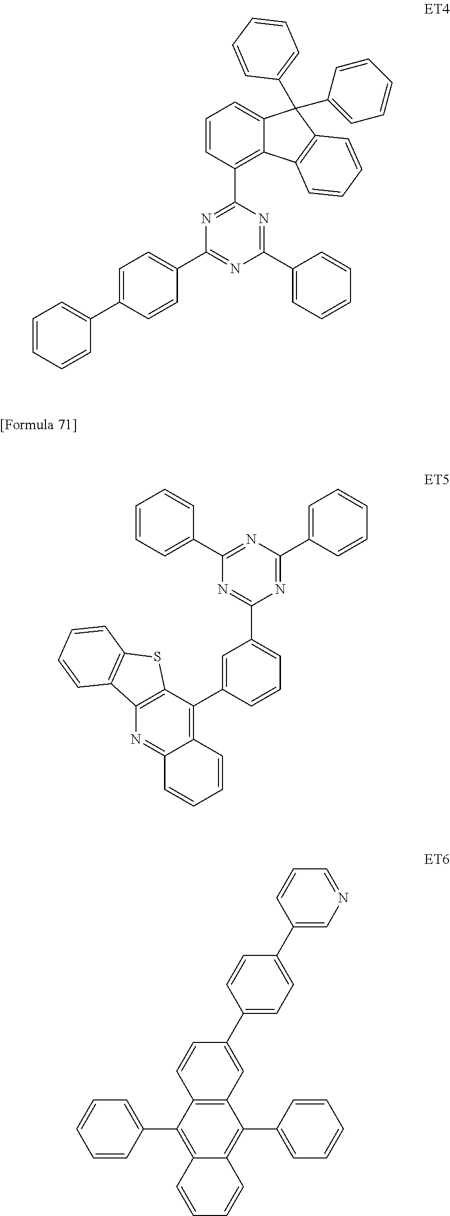 [SMILES: c1ccc(-c2ccc(-c3nc(-c4ccccc4)nc(-c4cccc5c4-c4ccccc4C5(c4ccccc4)c4ccccc4)n3)cc2)cc1 ; c1ccc(-c2nc(-c3ccccc3)nc(-c3cccc(-c4c5ccccc5nc5c4sc4ccccc45)c3)n2)cc1 ; c1ccc(-c2c3ccccc3c(-c3ccccc3)c3cc(-c4ccc(-c5cccnc5)cc4)ccc23)cc1]

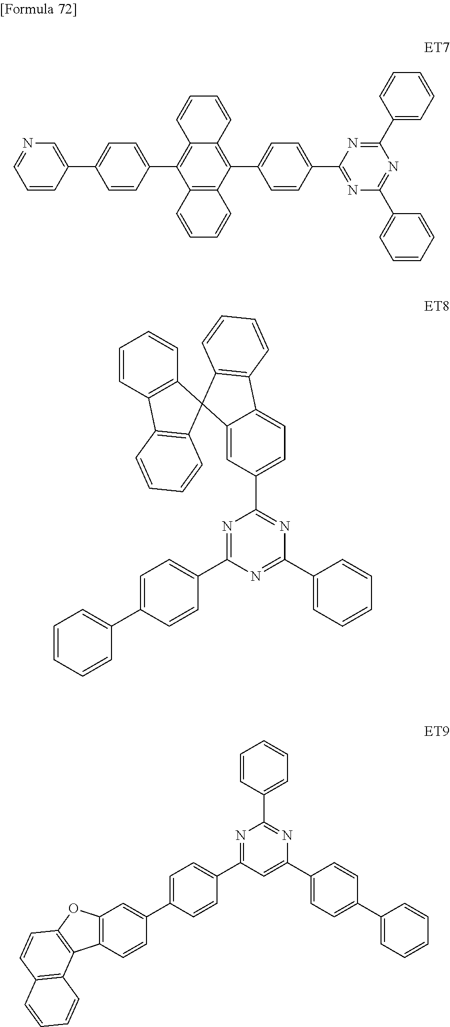 [SMILES: c1ccc(-c2nc(-c3ccccc3)nc(-c3ccc(-c4c5ccccc5c(-c5ccc(-c6cccnc6)cc5)c5ccccc45)cc3)n2)cc1 ; c1ccc(-c2ccc(-c3nc(-c4ccccc4)nc(-c4ccc5c(c4)C4(c6ccccc6-c6ccccc64)c4ccccc4-5)n3)cc2)cc1 ; c1ccc(-c2ccc(-c3cc(-c4ccc(-c5ccc6c(c5)oc5ccc7ccccc7c56)cc4)nc(-c4ccccc4)n3)cc2)cc1]

A compound used for the manufacturing organic EL devices in Examples and Comparatives is shown below.

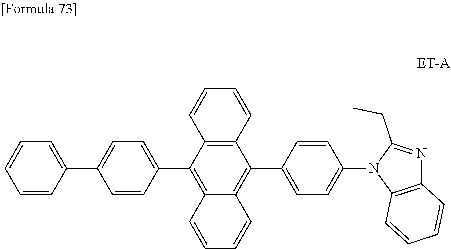 [SMILES: CCc1nc2ccccc2n1-c1ccc(-c2c3ccccc3c(-c3ccc(-c4ccccc4)cc3)c3ccccc23)cc1]

Structures of other compounds used for manufacturing the organic EL devices in Examples and Comparatives are shown below.

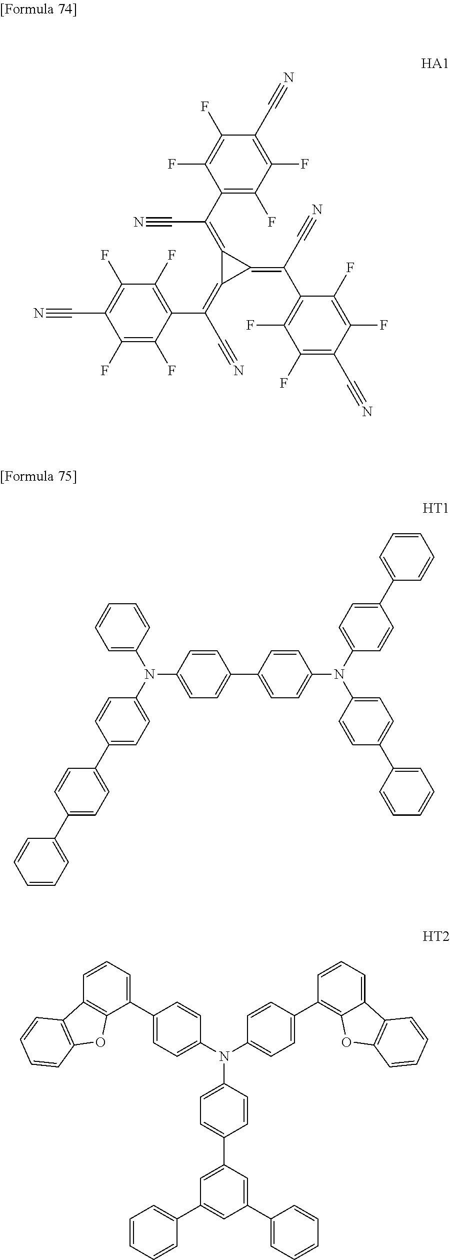 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(-c2cc(-c3ccccc3)cc(-c3ccc(N(c4ccc(-c5cccc6c5oc5ccccc56)cc4)c4ccc(-c5cccc6c5oc5ccccc56)cc4)cc3)c2)cc1]

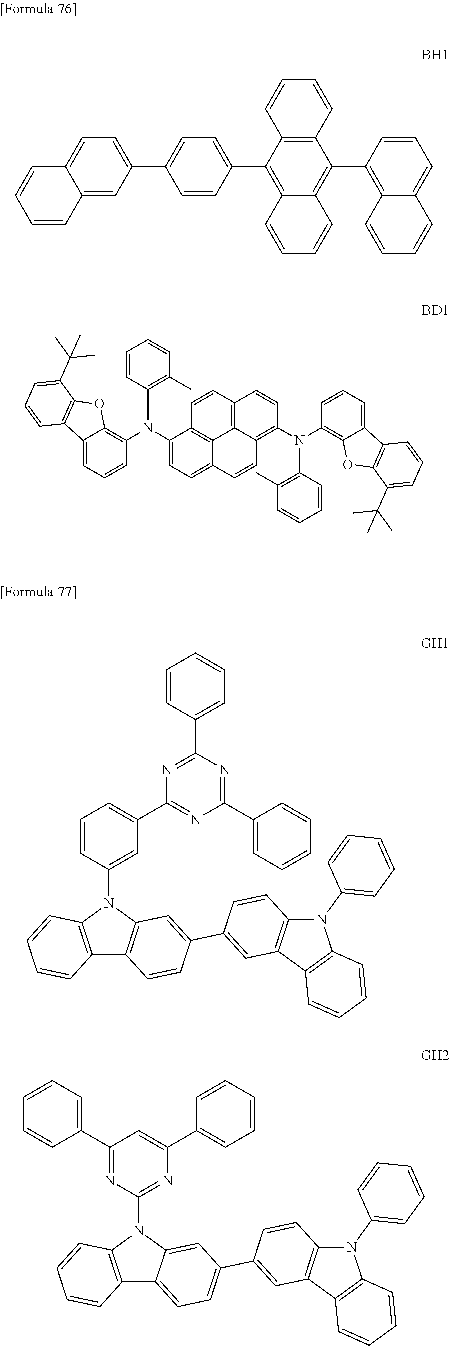

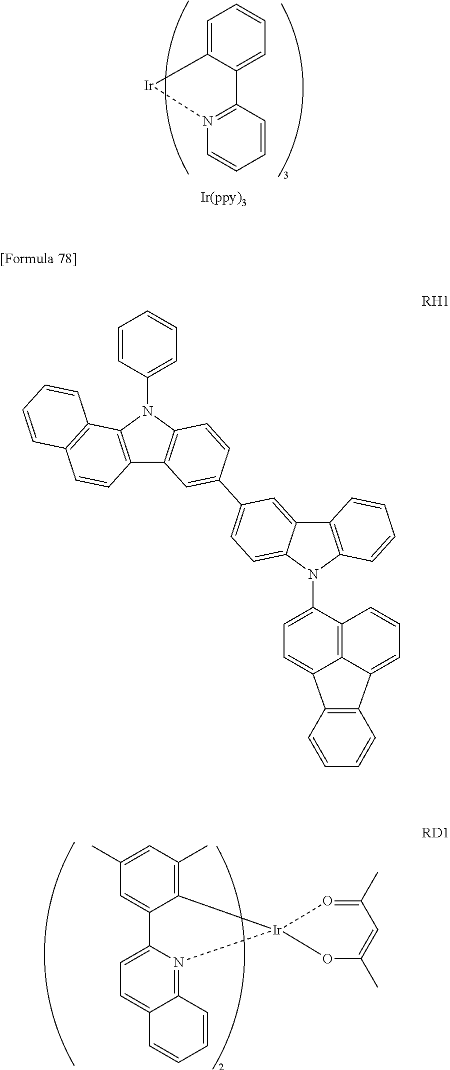

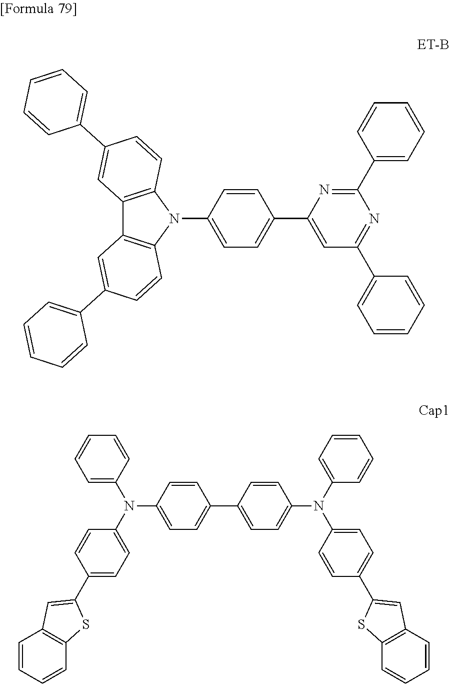 [SMILES: c1ccc(-c2ccc3c(c2)c2cc(-c4ccccc4)ccc2n3-c2ccc(-c3cc(-c4ccccc4)nc(-c4ccccc4)n3)cc2)cc1 ; c1ccc(N(c2ccc(-c3ccc(N(c4ccccc4)c4ccc(-c5cc6ccccc6s5)cc4)cc3)cc2)c2ccc(-c3cc4ccccc4s3)cc2)cc1]

### Manufacture 1 of Organic EL Device

The organic EL devices were manufactured and evaluated as follows.

### Example 1

A 200-nm-thick silver (Ag) layer as a light reflection layer and a 10-nm-thick ITO (Indium Tin Oxide) layer as a transparent electrode were sequentially formed by sputtering on a glass substrate (size: 25 mm×75 mm×0.7 mm thick) as a substrate for manufacturing a device. A lower electrode (anode) including the Ag layer and the ITO layer was thus formed.

Next, a compound HT1 and a compound HA1 were co-deposited on the ITO layer of the anode to form a 10-nm-thick hole injecting layer (HIL). The ratios of the compound HT1 and the compound HA1 in the hole injecting layer were 97 mass % and 3 mass %, respectively.

After the formation of the hole injecting layer, a compound HT2 was vapor-deposited to form a 10-nm-thick hole transporting layer (HTL).

After the formation of the hole transporting layer, a compound BH1 and a compound BD1 were co-deposited such that the ratio of the compound BD1 accounted for 3 mass %, thereby forming a 20-nm-thick emitting layer.

After the formation of the emitting layer, a compound ET1 was vapor-deposited to form a 140-nm-thick electron transporting layer (also referred to as a hole blocking layer (ETL1)).

After the formation of the electron transporting layer (ETL1), a compound ET-A was vapor-deposited to form a 10-nm-thick electron transporting layer (ETL2).

After the formation of the electron transporting layer (ETL2), LiF was vapor-deposited to form a 1-nm-thick electron injecting layer.

After the formation of the electron injecting layer, Mg and Ag were co-deposited to form a 15-nm-thick semitransmissive upper electrode (cathode) formed of a Mg—Ag alloy. A mixing ratio (film thickness ratio) of Mg to Ag in the upper electrode (cathode) was 15:85.

A compound Cap1 was vapor-deposited on the upper electrode to form a 65-nm-thick capping layer.

The organic EL device of Example 1 was manufactured as described above.

A device arrangement of the organic EL device in Example 1 is roughly shown as follows.

Ag (200)/ITO (10)/HT1:HA1 (10, 97%:3%)/HT2 (10)/BH1:BD1 (20, 97%:3%)/ET1 (140)/ET-A (10)/LiF (1)/Mg:Ag (15)/Cap1 (65)

Numerals in parentheses represent a film thickness (unit: nm).

The numerals (97%:3%) represented by percentage in the same parentheses indicate a ratio (mass %) between the compound HT1 and the compound HA1 in the hole injecting layer or a ratio (mass %) between the compound BH1 and the compound BD1 in the emitting layer. Hereinafter, a device arrangement may be roughly shown in the same manner.

### Examples 2 to 6

The organic EL devices of Examples 2 to 6 were each manufactured in the same manner as that of Example 1 except that the electron transporting layer (ETL1) of Example 1 was replaced by the electron transporting layer (ETL1) shown in Table 1.

### Example 7

The organic EL device of Example 7 was manufactured in the same manner as that of Example 1 except that the formation of the electron transporting layer (ETL1) of Example 1 was followed by co-depositing the compound ET1 and Liq to form a 10-nm-thick electron transporting layer (ETL2). The ratios of the compound ET1 and Liq in the electron transporting layer (ETL2) in Example 7 were 50 mass % and 50 mass %, respectively. Liq is an abbreviation of (8-Quinolinolato)lithium.

### Examples 8 to 10

The organic EL devices of Examples 8 to 10 were each manufactured in the same manner as that of Example 1 except that the electron transporting layer (ETL1) of Example 1 was replaced by the electron transporting layer (ETL1) shown in Table 1.

### Comparative 1

The organic EL device of Comparative 1 was manufactured in the same manner as that of Example 1 except that the formation of the emitting layer of Example 1 was followed by vapor-depositing the compound ET-A to form a 150-nm-thick electron transporting layer (ETL1) and the formation of the electron transporting layer (ETL1) was followed by vapor-depositing LiF without forming the electron transporting layer (ETL2).

### Comparative 2

The organic EL device of Comparative 2 was manufactured in the same manner as that of Example 1 except that the formation of the emitting layer of Example 1 was followed by co-depositing the compound ET-A and Liq to form a 140-nm-thick electron transporting layer (ETL1) and the formation of the electron transporting layer (ETL1) was followed by vapor-depositing the compound ET-A to form a 10-nm-thick electron transporting layer (ETL2). The ratios of the compound ET-A and Liq in the electron transporting layer (ETL1) in Comparative 2 were 50 mass % and 50 mass %, respectively.

### Evaluation 1 of Organic EL Devices

The manufactured organic EL devices were evaluated as follows. Table 1 shows evaluation results.

### Drive Voltage

The voltage (unit: V) when electric current was applied between the anode and the cathode so that the current density was 10 mA/cm² was measured.

Main Peak Wavelength Δp when Device is Driven

Voltage was applied on the organic EL devices so that a current density of the organic EL device was 10 mA/cm², where spectral radiance spectrum was measured by a spectroradiometer CS-2000 (manufactured by Konica Minolta, Inc.). The main peak wavelength Δp (unit: nm) was calculated based on the obtained spectral radiance spectrum.

TABLE 1

| | Electron Transporting | | Electron Transporting | | Voltage | |
|---|---|---|---|---|---|---|
| | Layer (ETL1) | | Layer (ETL2) | | (@10 mA/ | |
| | | Thickness | | Thickness | cm²) | λp |
| | Compound | [nm] | Compound | [nm] | [V] | [nm] |
| Example 1 | ET1 | 140 | ET-A | 10 | 3.36 | 457 |
| Example 2 | ET2 | 140 | ET-A | 10 | 3.36 | 457 |
| Example 3 | ET3 | 140 | ET-A | 10 | 3.23 | 457 |
| Example 4 | ET4 | 140 | ET-A | 10 | 3.33 | 457 |
| Example 5 | ET5 | 140 | ET-A | 10 | 3.70 | 457 |
| Example 6 | ET6 | 140 | ET-A | 10 | 3.93 | 457 |
| Example 7 | ET1 | 140 | ET1 and Liq | 10 | 3.50 | 457 |
| Example 8 | ET7 | 140 | ET-A | 10 | 3.65 | 457 |
| Example 9 | ET8 | 140 | ET-A | 10 | 3.31 | 457 |
| Example 10 | ET9 | 140 | ET-A | 10 | 3.35 | 457 |
| Comparative 1 | ET-A | 150 | | 0 | 4.26 | 457 |
| Comparative 2 | ET-A and Liq | 140 | ET-A | 10 | 5.70 | 457 |

### Manufacture 2 of Organic EL Device

### Example 11

A 200-nm-thick silver (Ag) layer as a light reflection layer and a 10-nm-thick ITO (Indium Tin Oxide) layer as a transparent electrode were sequentially formed by sputtering on a glass substrate (size: 25 mm×75 mm×0.7 mm thick) as a substrate for manufacturing a device. A lower electrode (anode) including the Ag layer and the ITO layer was thus formed.

Next, the compound HT1 and the compound HA1 were co-deposited on the ITO layer of the anode to form a 10-nm-thick hole injecting layer (HIL). The ratios of the compound HT1 and the compound HA1 in the hole injecting layer were 97 mass % and 3 mass %, respectively.

After the formation of the hole injecting layer, the compound HT2 was vapor-deposited to form a 10-nm-thick hole transporting layer (HTL).

After the formation of the hole transporting layer, the compound BH1 and the compound BD1 were co-deposited such that the ratio of the compound BD1 accounted for 3 mass %, thereby forming a 20-nm-thick emitting layer.

After the formation of the emitting layer, a compound ET-B was vapor-deposited to form a 10-nm-thick electron transporting layer (also referred to as a hole blocking layer (ETL3)).

After the formation of the electron transporting layer (ETL3), a compound ET2 was vapor-deposited to form a 130-nm-thick electron transporting layer (ETL1).

After the formation of the electron transporting layer (ETL1), the compound ET-A was vapor-deposited to form a 10-nm-thick electron transporting layer (ETL2).

After the formation of the electron transporting layer (ETL2), LiF was vapor-deposited to form a 1-nm-thick electron injecting layer.

After the formation of the electron injecting layer, Mg and Ag were co-deposited to form a 15-nm-thick semitransmissive upper electrode (cathode) formed of a Mg—Ag alloy. A mixing ratio (film thickness ratio) of Mg to Ag in the upper electrode (cathode) was 15:85.

The compound Cap1 was vapor-deposited on the upper electrode to form a 65-nm-thick capping layer.

The organic EL device of Example 11 was manufactured as described above.

A device arrangement of the organic EL device in Example 11 is roughly shown as follows.

Ag (200)/ITO (10)/HT1:HA1 (10, 97%:3%)/HT2 (10)/BH1:BD1 (20, 97%:3%)/ET-B (10)/ET2 (130)/ET-A (10)/LiF (1)/Mg:Ag (15)/Cap1 (65)

Numerals in parentheses represent a film thickness (unit: nm).

The numerals (97%:3%) represented by percentage in the same parentheses indicate a ratio (mass %) between the compound HT1 and the compound HA1 in the hole injecting layer or a ratio (mass %) between the compound BH1 and the compound BD1 in the emitting layer. Hereinafter, a device arrangement may be roughly shown in the same manner.

### Evaluation 2 of Organic EL Devices

The manufactured organic EL devices were evaluated as follows. Table 2 shows evaluation results. The evaluation results of Comparatives 1 and 2 are again shown in Table 2.

### Drive Voltage

The voltage (unit: V) when electric current was applied between the anode and the cathode so that the current density was 10 mA/cm² was measured.

Main Peak Wavelength Δp when Device is Driven

The main peak wavelength Δp (unit: nm) was calculated in the same manner as in the “Evaluation 1 of Organic EL Devices” described above.

TABLE 2

| | Electron | | Electron | | Electron | | | |
|---|---|---|---|---|---|---|---|---|
| | Transporting | | Transporting | | Transporting | | Voltage | |
| | Layer (ETL3) | | Layer (ETL1) | | Layer (ETL2) | | (@10 mA/ | |
| | Com- | Thickness | | Thickness | Com- | Thickness | cm²) | λp |
| | pound | [nm] | Compound | [nm] | pound | [nm] | [V] | [nm] |
| Example 11 | ET-B | 10 | ET2 | 130 | ET-A | 10 | 3.50 | 457 |
| Comparative 1 | | 0 | ET-A | 150 | | 0 | 4.26 | 457 |
| Comparative 2 | | 0 | ET-A and | 140 | ET-A | 10 | 5.70 | 457 |
| | | | Liq | | | | | |

### Manufacture 3 of Organic EL Device

### Example 12

A 200-nm-thick silver (Ag) layer as a light reflection layer and a 10-nm-thick ITO (Indium Tin Oxide) layer as a transparent electrode were sequentially formed by sputtering on a glass substrate (size: 25 mm×75 mm×0.7 mm thick) as a substrate for manufacturing a device. A lower electrode (anode) including the Ag layer and the ITO layer was thus formed.

Next, the compound HT1 and the compound HA1 were co-deposited on the ITO layer of the anode to form a 10-nm-thick hole injecting layer (HIL). The ratios of the compound HT1 and the compound HA1 in the hole injecting layer were 97 mass % and 3 mass %, respectively.

After the formation of the hole injecting layer, the compound HT2 was vapor-deposited to form a 10-nm-thick hole transporting layer (HTL).

After the formation of the hole transporting layer, a compound GH1, a compound GH2 and a compound Ir(ppy)₃ were co-deposited to form a 40-nm-thick emitting layer. The ratios of the compound GH1, the compound GH2 and the compound Ir(ppy)₃ in the emitting layer were 45 mass %, 50 mass % and 5 mass %, respectively.

After the formation of the emitting layer, the compound ET1 was vapor-deposited to form a 180-nm-thick electron transporting layer (also referred to as a hole blocking layer (ETL1)).

After the formation of the electron transporting layer (ETL1), the compound ET-A was vapor-deposited to form a 10-nm-thick electron transporting layer (ETL2).

After the formation of the electron transporting layer (ETL2), LiF was vapor-deposited to form a 1-nm-thick electron injecting layer.

After the formation of the electron injecting layer, Mg and Ag were co-deposited to form a 15-nm-thick semitransmissive upper electrode (cathode) formed of a Mg—Ag alloy. A mixing ratio (film thickness ratio) of Mg to Ag in the upper electrode (cathode) was 15:85.

The compound Cap1 was vapor-deposited on the upper electrode to form a 65-nm-thick capping layer.

The organic EL device of Example 12 was manufactured as described above.

A device arrangement of the organic EL device in Example 12 is roughly shown as follows.

Ag (200)/ITO (10)/HT1:HA1 (10, 97%:3%)/HT2 (10)/GH1:GH2:Ir(ppy)₃ (40, 45%:50%:5%)/ET1 (180)/ET-A (10)/LiF (1)/Mg:Ag (15)/Cap1 (65)

Numerals in parentheses represent a film thickness (unit: nm).

The numerals (97%:3%) represented by percentage in the same parentheses indicate a ratio (mass %) between the compound HT1 and the compound HA1 in the hole injecting layer. The numerals (45%:50%:5%) represented by percentage in the same parentheses indicate a ratio (mass %) between the compound GH1, the compound GH2 and Ir(ppy)₃ in the emitting layer. Hereinafter, a device arrangement may be roughly shown in the same manner.

### Example 13

The organic EL device of Example 13 was manufactured in the same manner as that of Example 12 except that the electron transporting layer (ETL1) of Example 12 was replaced by the electron transporting layer (ETL1) shown in Table 3.

### Comparative 3

The organic EL device of Comparative 3 was manufactured in the same manner as that of Example 12 except that the formation of the emitting layer of Example 12 was followed by vapor-depositing the compound ET-A to form a 190-nm-thick electron transporting layer (ETL1) and the formation of the electron transporting layer (ETL1) was followed by vapor-depositing LiF without forming the electron transporting layer (ETL2).

### Evaluation 3 of Organic EL Devices

The manufactured organic EL devices were evaluated as follows. Table 3 shows evaluation results.

### Drive Voltage

The voltage (unit: V) when electric current was applied between the anode and the cathode so that the current density was 10 mA/cm² was measured.

Main Peak Wavelength Δp when Device is Driven

The main peak wavelength Δp (unit: nm) was calculated in the same manner as in the “Evaluation 1 of Organic EL Devices” described above.

TABLE 3

| | Electron | | Electron | | | |
|---|---|---|---|---|---|---|
| | Transporting | | Transporting | | Voltage | |
| | Layer (ETL1) | | Layer (ETL2) | | (@10 | |
| | Com- | Thickness | Com- | Thickness | mA/cm²) | λp |
| | pound | [nm] | pound | [nm] | [V] | [nm] |
| Example 12 | ET1 | 180 | ET-A | 10 | 3.94 | 520 |
| Example 13 | ET2 | 180 | ET-A | 10 | 4.00 | 520 |
| Compara- | ET-A | 190 | | 0 | 5.36 | 520 |
| tive 3 | | | | | | |

### Manufacture 4 of Organic EL Device

### Example 14

A 200-nm-thick silver (Ag) layer as a light reflection layer and a 10-nm-thick ITO (Indium Tin Oxide) layer as a transparent electrode were sequentially formed by sputtering on a glass substrate (size: 25 mm×75 mm×0.7 mm thick) as a substrate for manufacturing a device. A lower electrode (anode) including the Ag layer and the ITO layer was thus formed.

Next, the compound HT1 and the compound HA1 were co-deposited on the ITO layer of the anode to form a 10-nm-thick hole injecting layer (HIL). The ratios of the compound HT1 and the compound HA1 in the hole injecting layer were 97 mass % and 3 mass %, respectively.

After the formation of the hole injecting layer, the compound HT2 was vapor-deposited to form a 10-nm-thick hole transporting layer (HTL).

After the formation of the hole transporting layer, a compound RH1 and a compound RD1 were co-deposited to form a 40-nm-thick emitting layer. The ratios of the compound RH1 and the compound RD1 in the emitting layer were 95 mass % and 5 mass %, respectively.

After the formation of the emitting layer, the compound ET1 was vapor-deposited to form a 220-nm-thick electron transporting layer (also referred to as a hole blocking layer (ETL1)).

After the formation of the electron transporting layer (ETL1), the compound ET-A was vapor-deposited to form a 10-nm-thick electron transporting layer (ETL2).

After the formation of the electron transporting layer (ETL2), LiF was vapor-deposited to form a 1-nm-thick electron injecting layer.

After the formation of the electron injecting layer, Mg and Ag were co-deposited to form a 15-nm-thick semitransmissive upper electrode (cathode) formed of a Mg—Ag alloy. A mixing ratio (film thickness ratio) of Mg to Ag in the upper electrode (cathode) was 15:85.

The compound Cap1 was vapor-deposited on the upper electrode to form a 65-nm-thick capping layer.

The organic EL device of Example 14 was manufactured as described above.

A device arrangement of the organic EL device in Example 14 is roughly shown as follows.

Ag (200)/ITO (10)/HT1:HA1 (10, 97%:3%)/HT2 (10)/RH1:RD1 (40, 95%:5%)/ET1 (220)/ET-A (10)/LiF (1)/Mg:Ag (15)/Cap1 (65)

Numerals in parentheses represent a film thickness (unit: nm).

The numerals (97%:3%) represented by percentage in the same parentheses indicate a ratio (mass %) between the compound HT1 and the compound HA1 in the hole injecting layer. The numerals (95%:5%) represented by percentage in the same parentheses indicate a ratio (mass %) between the compound RH1 and the compound RD1 in the emitting layer. Hereinafter, a device arrangement may be roughly shown in the same manner.

### Example 15

The organic EL device of Example 15 was manufactured in the same manner as that of Example 14 except that the electron transporting layer (ETL1) of Example 14 was replaced by the electron transporting layer (ETL1) shown in Table 4.

### Comparative 4

The organic EL device of Comparative 4 was manufactured in the same manner as that of Example 14 except that the formation of the emitting layer of Example 14 was followed by vapor-depositing the compound ET-A to form a 190-nm-thick electron transporting layer (ETL1) and the formation of the electron transporting layer (ETL1) was followed by vapor-depositing LiF without forming the electron transporting layer (ETL2).

### Evaluation 4 of Organic EL Devices

The manufactured organic EL devices were evaluated as follows. Table 4 shows evaluation results.

### Drive Voltage

The voltage (unit: V) when electric current was applied between the anode and the cathode so that the current density was 10 mA/cm² was measured.

Main Peak Wavelength Δp when Device is Driven

The main peak wavelength Δp (unit: nm) was calculated in the same manner as in the “Evaluation 1 of Organic EL Devices” described above.

TABLE 4

| | Electron | | Electron | | | |
|---|---|---|---|---|---|---|
| | Transporting | | Transporting | | Voltage | |
| | Layer (ETL1) | | Layer (ETL2) | | (@10 | |
| | Com- | Thickness | Com- | Thickness | mA/cm²) | λp |
| | pound | [nm] | pound | [nm] | [V] | [nm] |
| Example 14 | ET1 | 220 | ET-A | 10 | 3.84 | 620 |
| Example 15 | ET2 | 220 | ET-A | 10 | 4.01 | 620 |
| Compara- | ET-A | 190 | | 0 | 5.40 | 620 |
| tive 4 | | | | | | |

### EXPLANATION OF CODES

1, 1A, 1B, 1C . . . organic EL device, 2 . . . substrate, 3 . . . anode, 4 . . . semitransmissive electrode, 5 . . . emitting layer, 6 . . . hole transporting zone, 7, 7A, 7B, 7C . . . electron transporting zone, 8 . . . capping layer, 10 . . . organic layer, 31 . . . light reflection layer, 32 . . . transparent electrode, 61 . . . hole injecting layer, 62 . . . hole transporting layer, 71 . . . first layer, 72 . . . electron injecting layer, 73 . . . second layer, 74 . . . third layer, P1 . . . first end, P2 . . . second end

1. An organic electroluminescence device comprising:
an emitting layer between an anode and a cathode; and
a first layer between the cathode and the emitting layer, wherein
the first layer has a thickness of 50 nm or more,
the first layer comprises a compound represented by a formula (100) below, and
the first layer comprises no metal doping material, 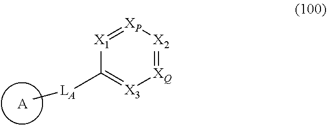
where:
A is a substituted or unsubstituted fused aryl group having 13 to 50 ring carbon atoms, or a substituted or unsubstituted fused heterocyclic group having 14 to 50 ring atoms;
L_{A} is a single bond, a substituted or unsubstituted arylene group having 6 to 30 ring carbon atoms, or a substituted or unsubstituted divalent heterocyclic group having 5 to 30 ring atoms;
X₁, X₂ and X₃ are each independently a nitrogen atom or CR₃;
X_{P} is a nitrogen atom or CR₁;
X_{Q} is a nitrogen atom or CR₂;
at least one of X₁, X₂, X₃, X_{P} or X_{Q} is a nitrogen atom;
at least one combination of adjacent two or more of R₁, R₂ and R₃ are mutually bonded to form a substituted or unsubstituted monocyclic ring, mutually bonded to form a substituted or unsubstituted fused ring, or not mutually bonded;
R₁, R₂ and R₃ not forming the substituted or unsubstituted monocyclic ring and not forming the substituted or unsubstituted fused ring are each independently a hydrogen atom, a substituted or unsubstituted aryl group having 6 to 30 ring carbon atoms, or a substituted or unsubstituted heterocyclic group having 5 to 30 ring atoms; and
when a plurality of R₃ are present, the plurality of R₃ are mutually the same or different.

2. The organic electroluminescence device according to claim 1, wherein
the compound represented by the formula (100) is a compound represented by a formula (1) below, 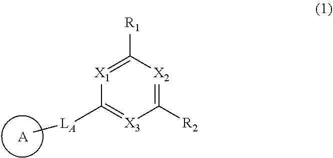
where:
A is a substituted or unsubstituted fused aryl group having 13 to 50 ring carbon atoms, or a substituted or unsubstituted fused heterocyclic group having 14 to 50 ring atoms;
L_{A} is a single bond, a substituted or unsubstituted arylene group having 6 to 30 ring carbon atoms, or a substituted or unsubstituted divalent heterocyclic group having 5 to 30 ring atoms;
X₁, X₂ and X₃ are each independently a nitrogen atom or CR₃;
at least one of X₁, X₂ or X₃ is a nitrogen atom;
at least one combination of adjacent two or more of R₁, R₂ and R₃ are mutually bonded to form a substituted or unsubstituted monocyclic ring, mutually bonded to form a substituted or unsubstituted fused ring, or not mutually bonded;
R₁, R₂ and R₃ not forming the substituted or unsubstituted monocyclic ring and not forming the substituted or unsubstituted fused ring are each independently a hydrogen atom, a substituted or unsubstituted aryl group having 6 to 30 ring carbon atoms, or a substituted or unsubstituted heterocyclic group having 5 to 30 ring atoms; and
when a plurality of R₃ are present, the plurality of R₃ are mutually the same or different.

3. The organic electroluminescence device according to claim 1, wherein
the compound represented by the formula (100) is a compound represented by a formula (101) below, 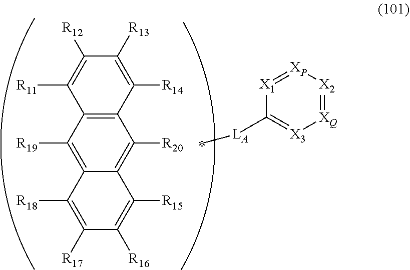
where:
X₁ to X₃, X_{P}, X_{Q}, R₁ to R₃ and L_{A} each represent the same as defined in the formula (100);
one of R₁₁ to R₂₀ is a bonding position * to L_{A};
at least one combination of adjacent two or more of R₁₁ to R₂₀ not being the bonding position to L_{A} are mutually bonded to form a substituted or unsubstituted monocyclic ring, mutually bonded to form a substituted or unsubstituted fused ring, or not mutually bonded;
R₁₁ to R₂₀ not being the bonding position to L_{A}, not forming the substituted or unsubstituted monocyclic ring and not forming the substituted or unsubstituted fused ring each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅) a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms:
R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms:
when a plurality of R₉₀₁ are present, the plurality of R₉₀₁ are mutually the same or different:
when a plurality of R₉₀₂ are present, the plurality of R₉₀₂ are mutually the same or different:
when a plurality of R₉₀₃ are present, the plurality of R₉₀₃ are mutually the same or different:
when a plurality of R₉₀₄ are present, the plurality of R₉₀₄ are mutually the same or different:
when a plurality of R₉₀₅ are present, the plurality of R₉₀₅ are mutually the same or different:
when a plurality of R₉₀₆ are present, the plurality of R₉₀₆ are mutually the same or different:
when a plurality of R₉₀₇ are present, the plurality of R₉₀₇ are mutually the same or different:
when a plurality of R₈₀₁ are present, the plurality of R₈₀₁ are mutually the same or different; and
when a plurality of R₈₀₂ are present, the plurality of R₈₀₂ are mutually the same or different.

4. The organic electroluminescence device according to claim 2, wherein
the compound represented by the formula (1) is a compound represented by a formula (A1) below, 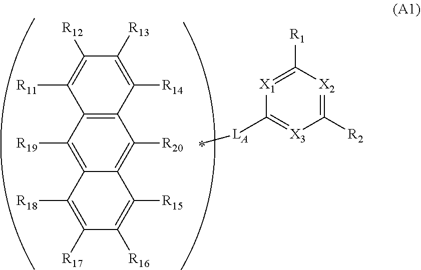
where:
X₁ to X₃, R₁ to R₃ and L_{A} each represent the same as defined in the formula (1);
one of R₁₁ to R₂₀ is a bonding position * to L_{A};
at least one combination of adjacent two or more of R₁₁ to R₂₀ not being the bonding position to L_{A} are mutually bonded to form a substituted or unsubstituted monocyclic ring, mutually bonded to form a substituted or unsubstituted fused ring, or not mutually bonded;
R₁₁ to R₂₀ not being the bonding position to L_{A}, not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

5. The organic electroluminescence device according to claim 3, wherein
two or more of R₁₁ to R₂₀ not being the bonding position to L_{A}, not forming the substituted or unsubstituted monocyclic ring and not forming the substituted or unsubstituted fused ring are each not a hydrogen atom.

6. The organic electroluminescence device according to claim 3, wherein
two or more of R₁₁ to R₂₀ not being the bonding position to L_{A}, not forming the substituted or unsubstituted monocyclic ring and not forming the substituted or unsubstituted fused ring are each independently a substituted or unsubstituted aryl group having 6 to 50 ring carbon atoms, or a substituted or unsubstituted heterocyclic group having 5 to 50 ring atoms.

7. The organic electroluminescence device according to claim 3, wherein
R₁₉ and R₂₀ are each independently a substituted or unsubstituted aryl group having 6 to 50 ring carbon atoms, or a substituted or unsubstituted heterocyclic group having 5 to 50 ring atoms.

8. The organic electroluminescence device according to claim 4, wherein
the compound represented by the formula (100) is a compound represented by a formula (A1-1) below, 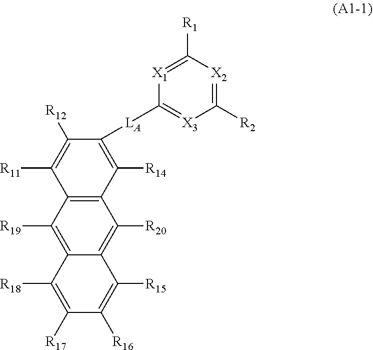
where:
X₁ to X₃, R₁ to R₃ and L_{A} each represent the same as defined in the formula (1);
at least one combination of adjacent two or more of R₁₁, R₁₂ and R₁₄ to R₂₀ are mutually bonded to form a substituted or unsubstituted monocyclic ring, mutually bonded to form a substituted or unsubstituted fused ring, or not mutually bonded;
R₁₁, R₁₂ and R₁₄ to R₂₀ not being the bonding position to L_{A}, not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
R₉₀₁, R₉₀₂, R₉₀₃, R₉₀₄, R₉₀₅, R₉₀₆, R₉₀₇, R₈₀₁ and R₈₀₂ each represent the same as defined in the formula (A1).

9. The organic electroluminescence device according to claim 4, wherein
the compound represented by the formula (100) is a compound represented by a formula (A1-2) below, 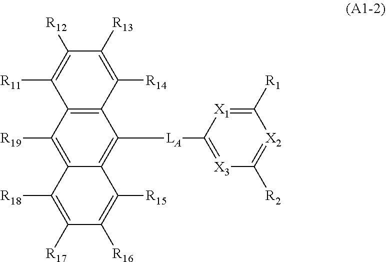
where:
X₁ to X₃, R₁ to R₃ and L_{A} each represent the same as defined in the formula (1);
at least one combination of adjacent two or more of R₁₁ to R₁₉ are mutually bonded to form a substituted or unsubstituted monocyclic ring, mutually bonded to form a substituted or unsubstituted fused ring, or not mutually bonded;
R₁₁ to R₁₉ not being the bonding position to L_{A}, not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
R₉₀₁, R₉₀₂, R₉₀₃, R₉₀₄, R₉₀₅, R₉₀₆, R₉₀₇, R₈₀₁ and R₈₀₂ each represent the same as defined in the formula (A1).

10. The organic electroluminescence device according to claim 9, wherein
R₁₉ is a substituted or unsubstituted aryl group having 6 to 50 ring carbon atoms, or a substituted or unsubstituted heterocyclic group having 5 to 50 ring atoms.

11. The organic electroluminescence device according to claim 2, wherein
the compound represented by the formula (1) is a compound represented by a formula (B1) below, 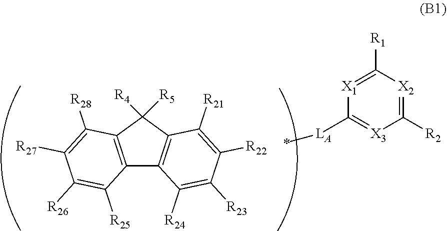
where:
X₁ to X₃, R₁ to R₃ and L_{A} each represent the same as defined in the formula (1);
one of R₂₁ to R₂₈ is a bonding position * to L_{A};
R₂₁ to R₂₈ not being the bonding position to L_{A} are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
a combination of R₄ and R₅ are mutually bonded to form a substituted or unsubstituted monocyclic ring, mutually bonded to form a substituted or unsubstituted fused ring, or not mutually bonded;
R₄ and R₅ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms;
R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

12. The organic electroluminescence device according to claim 11, wherein
R₄ and R₅ are each independently a substituted or unsubstituted aryl group having 6 to 50 ring carbon atoms.

13. The organic electroluminescence device according to claim 11, wherein
R₄ and R₅ are each independently a substituted or unsubstituted phenyl group.

14. The organic electroluminescence device according to claim 11, wherein
the compound represented by the formula (B1) is a compound represented by a formula (B1-1) below, 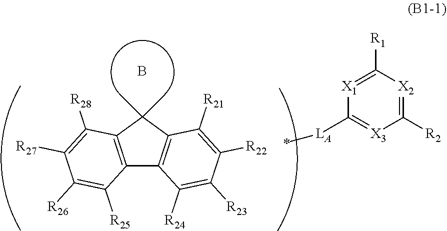
where:
X₁ to X₃, R₁ to R₃ and L_{A} each represent the same as defined in the formula (1);
R₂₁ to R₂₈ each represent the same as defined in the formula (B1); and
a ring B is a substituted or unsubstituted monocyclic ring or a substituted or unsubstituted fused ring.

15. The organic electroluminescence device according to claim 14, wherein
the compound represented by the formula (B1) is a compound represented by a formula (B1-2) below, 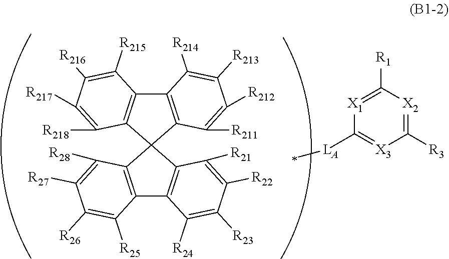
where:
X₁ to X₃, R₁ to R₃ and L_{A} each represent the same as defined in the formula (1);
R₂₁ to R₂₈ each represent the same as defined in the formula (B1-1);
at least one combination of adjacent two or more of R₂₁₁ to R₂₁₈ are mutually bonded to form a substituted or unsubstituted monocyclic ring, mutually bonded to form a substituted or unsubstituted fused ring, or not mutually bonded;
R₂₁₁ to R₂₁₈ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
R₉₀₁, R₉₀₂, R₉₀₃, R₉₀₄, R₉₀₅, R₉₀₆, R₉₀₇, R₈₀₁ and R₈₀₂ each represent the same as defined in the formula (B1).

16. The organic electroluminescence device according to claim 1, wherein
A is a substituted or unsubstituted fused aryl group having 13 to 30 ring carbon atoms, or a substituted or unsubstituted fused heterocyclic group having 14 to 30 ring atoms.

17. The organic electroluminescence device according to claim 1, wherein
A is a substituted or unsubstituted fused aryl group having 13 to 20 ring carbon atoms, or a substituted or unsubstituted fused heterocyclic group having 14 to 20 ring atoms.

18. The organic electroluminescence device according to claim 1, wherein
A is a substituted or unsubstituted fused heterocyclic group having 14 to 20 ring atoms.

19. The organic electroluminescence device according to claim 1, wherein
A is a fused heterocyclic group including two or more heteroatoms as ring atoms.

20. The organic electroluminescence device according to claim 1, wherein
one of X₁, X₂ and X₃ is a nitrogen atom.

21. The organic electroluminescence device according to claim 1, wherein
X₂ is a nitrogen atom,
X₁ and X₃ are each CR₃,
R₃ represents the same as defined in the formula (10M), and
two R₃ are mutually the same or different.

22. The organic electroluminescence device according to claim 1, wherein
X₁, X₂ and X₃ are each a nitrogen atom.

23. The organic electroluminescence device according to claim 1, wherein
R₁ and R₂ are each independently a substituted or unsubstituted aryl group having 6 to 50 ring carbon atoms.

24. The organic electroluminescence device according to claim 1, wherein
R₁ and R₂ are each independently a substituted or unsubstituted aryl group having 6 to 30 ring carbon atoms.

25. The organic electroluminescence device according to claim 1, wherein
R₁ and R₂ are each independently a substituted or unsubstituted aryl group having 6 to 18 ring carbon atoms.

26. The organic electroluminescence device according to claim 1, wherein
L_{A} is a single bond.

27. The organic electroluminescence device according to claim 1, wherein
L_{A} is a divalent group represented by a formula (L1-1), (L1-2) or (L1-3) below, 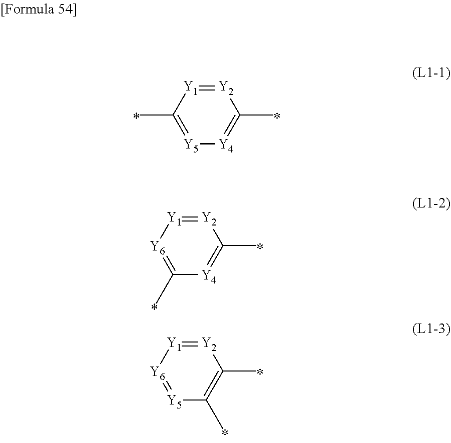
where:
Y₁ to Y₆ are each independently a nitrogen atom or CR₆;
R₆ is a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
when a plurality of R₆ are present, the plurality of R₆ are mutually the same or different;
* represents a bonding position;
R₉₀₁, R₉₀₂, R₉₀₃, R₉₀₄, R₉₀₅, R₉₀₆, R₉₀₇, R₈₀₁ and R₉₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

28. The organic electroluminescence device according to claim 27, wherein
Y₁ to Y₆ are each CR₆, and
R₆ is a hydrogen atom.

29. The organic electroluminescence device according to claim 1, wherein
all groups described as “substituted or unsubstituted” groups in the compound represented by the formula (100) are “unsubstituted” groups.

30. The organic electroluminescence device according to claim 1, wherein
a distance D₁ between an interface of the cathode close to the emitting layer and an interface of the emitting layer close to the cathode is larger than a distance D₂ between an interface of the anode close to the emitting layer and an interface of the emitting layer close to the anode.

31. The organic electroluminescence device according to claim 1, wherein
the first layer has a thickness of 100 nm or more.

32. The organic electroluminescence device according to claim 1, wherein
the emitting layer and the first layer are in direct contact with each other.

33. The organic electroluminescence device according to claim 1, further comprising:
a second layer between the emitting layer and the first layer.

34. The organic electroluminescence device according to claim 1, further comprising:
a third layer between the cathode and the first layer.

35-37. (canceled)
